SEQUENCE LISTING (23 sequences)

SEQ ID NO: 1 - protein, 13 aa, Severe acute respiratory syndrome-related coronavirus
  source 1..13: note = Severe acute respiratory syndrome coronavirus 2
MFVFLVLLPLVSS

SEQ ID NO: 2 - protein, 197 aa, Severe acute respiratory syndrome-related coronavirus
  source 1..197: note = Severe acute respiratory syndrome coronavirus 2
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 3 - protein, 42 aa, Staphylococcus aureus
  source 1..42: note = S. aureus strain Mu50
IENADKAIKDFQDNKAPHDKSAAYEANSKLPKDLRDKNNRFV

SEQ ID NO: 4 - protein, 58 aa, Staphylococcus aureus
  source 1..58: note = S. aureus strain Mu50
EKVSIEKAIVRHDERVKSANDAISKLNEKDSIENRRLAQREVNKAPMDVKEHLQKQLD

SEQ ID NO: 5 - protein, 100 aa, Staphylococcus aureus
  source 1..100: note = S. aureus strain Mu50
IENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 6 - protein, 15 aa, synthetic construct
GGGGSGGGGSGGGGS

SEQ ID NO: 7 - protein, 212 aa, synthetic construct
MFVFLVLLPLVSSAANITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGP

SEQ ID NO: 8 - protein, 327 aa, synthetic construct
MFVFLVLLPLVSSAANITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPGGGGSGGGGSGGGGSIENADKAIKDFQDNKAPHDKSAAYEANSKLPKDLRDKNNRFVEKVSIEKAIVRHDERVKSANDAISKLNEKDSIENRRLAQREVNKAPMDVKEHLQKQLD

SEQ ID NO: 9 - DNA, 823 bp, synthetic construct
  source 1..823: mol_type = other DNA
cttgttctttttgcagaagctcagaataaacgctcaactttggccaccatgttcgtattcctcgtcttgcttcctctcgtctccagtgcagcaaacatcaccaacctctgcccttttggcgaagtgttcaacgccacacgctttgcctctgtgtacgcatggaataggaagagaatatctaactgcgttgccgattattctgtactctacaatagtgcatcattttccacgttcaagtgctatggggtgtctccaaccaaattgaacgatctgtgcttcaccaatgtgtatgcagatagctttgtaattagaggggacgaagtgcggcaaattgcccctggtcaaacgggcaaaatcgcagattataattataaactcccagatgattttactggctgcgtaatcgcatggaactcaaacaatcttgactctaaggtgggcgggaactataactatctctacagactgtttcgaaagtccaatctgaagccgtttgagcgggacatttccactgagatctatcaagccgggtccaccccgtgcaacggggtggaaggctttaactgttactttcctcttcagagttatgggttccaaccgactaatggtgtaggctatcagccataccgagtcgttgttctcagctttgagctgctgcatgcacctgctacagtttgcggcccataatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 10 - DNA, 1168 bp, synthetic construct
  source 1..1168: mol_type = other DNA
cttgttctttttgcagaagctcagaataaacgctcaactttggccaccatgttcgtattcctcgtcttgcttcctctcgtctccagtgcagcaaacatcaccaacctctgcccttttggcgaagtgttcaacgccacacgctttgcctctgtgtacgcatggaataggaagagaatatctaactgcgttgccgattattctgtactctacaatagtgcatcattttccacgttcaagtgctatggggtgtctccaaccaaattgaacgatctgtgcttcaccaatgtgtatgcagatagctttgtaattagaggggacgaagtgcggcaaattgcccctggtcaaacgggcaaaatcgcagattataattataaactcccagatgattttactggctgcgtaatcgcatggaactcaaacaatcttgactctaaggtgggcgggaactataactatctctacagactgtttcgaaagtccaatctgaagccgtttgagcgggacatttccactgagatctatcaagccgggtccaccccgtgcaacggggtggaaggctttaactgttactttcctcttcagagttatgggttccaaccgactaatggtgtaggctatcagccataccgagtcgttgttctcagctttgagctgctgcatgcacctgctacagtttgcggcccagggggtggaggatccggtggtggtggctctggaggaggaggcagcatagagaatgccgataaagctatcaaggactttcaggacaacaaagcaccgcacgataagagcgctgcttacgaggccaattcaaaattgccaaaagacctccgagacaagaacaaccggttcgttgagaaggttagtattgaaaaggccattgtgcgccatgacgaacgcgtcaagtctgccaacgatgctattagtaagctgaacgaaaaggattctatcgagaaccgaaggcttgctcaaagagaggttaacaaggcccctatggacgttaaagaacatttgcaaaagcaattggattaatgatagaccagcctcaagaacacccgaatggagtctctaagctacataataccaacttacactttacaaaatgttgtcccccaaaatgtagccattcgtatctgctcctaataaaaagaaagtttcttcacattct

SEQ ID NO: 11 - DNA, 45 bp, synthetic construct
  source 1..45: mol_type = other DNA
gaatttaatacgactcactataaggcttgttctttttgcagaagc

SEQ ID NO: 12 - DNA, 149 bp, synthetic construct
  source 1..149: mol_type = other DNA
ttttttttttttttttttttttttttttttttttttttttttttttttttttttttttttttttttttttttttttttttttttttttttttttttttttttttttttttttttttttttagaatgtgaagaaactttctttttattag

SEQ ID NO: 13 - DNA, 48 bp, synthetic construct
  source 1..48: mol_type = other DNA
gaatttaatacgactcactataaggataaactagtattcttctggtcc

SEQ ID NO: 14 - protein, 1273 aa, Severe acute respiratory syndrome-related coronavirus
  source 1..1273: note = Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3822 bp, Severe acute respiratory syndrome-related coronavirus
  source 1..3822: mol_type = other DNA; note = Severe acute respiratory syndrome coronavirus 2
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 16 - DNA, 1311 bp, Staphylococcus aureus
  source 1..1311: mol_type = other DNA; note = Staphylococcus aureus subsp. aureus NCTC 8325
atgaaaaataaatatatctcgaagttgctagttggggcagcaacaattacgttagctacaatgatttcaaatggggaagcaaaagcgagtgaaaacacgcaacaaacttcaactaagcaccaaacaactcaaaacaactacgtaacagatcaacaaaaagctttttatcaagtattacatctaaaaggtatcacagaagaacaacgtaaccaatacatcaaaacattacgcgaacacccagaacgtgcacaagaagtattctctgaatcacttaaagacagcaagaacccagaccgacgtgttgcacaacaaaacgctttttacaatgttcttaaaaatgataacttaactgaacaagaaaaaaataattacattgcacaaattaaagaaaaccctgatagaagccaacaagtttgggtagaatcagtacaatcttctaaagctaaagaacgtcaaaatattgaaaatgcggataaagcaattaaagatttccaagataacaaagcaccacacgataaatcagcagcatatgaagctaactcaaaattacctaaagatttacgtgataaaaacaaccgctttgtagaaaaagtttcaattgaaaaagcaatcgttcgtcatgatgagcgtgtgaaatcagcaaatgatgcaatctcaaaattaaatgaaaaagattcaattgaaaacagacgtttagcacaacgtgaagttaacaaagcacctatggatgtaaaagagcatttacagaaacaattagacgcattagttgctcaaaaagatgctgaaaagaaagtggcgccaaaagttgaggctcctcaaattcaatcaccacaaattgaaaaacctaaagtagaatcaccaaaagttgaagtccctcaaattcaatcaccaaaagttgaggttcctcaatctaaattattaggttactaccaatcattaaaagattcatttaactatggttacaagtatttaacagatacttataaaagctataaagaaaaatatgatacagcaaagtactactataatacgtactataaatacaaaggtgcgattgatcaaacagtattaacagtactaggtagtggttctaaatcttacatccaaccattgaaagttgatgataaaaacggctacttagctaaatcatatgcacaagtaagaaactatgtaactgagtcaatcaatactggtaaagtattatatactttctaccaaaacccaacattagtaaaaacagctattaaagctcaagaaactgcatcatcaatcaaaaatacattaagtaatttattatcattctggaaataa

SEQ ID NO: 17 - protein, 436 aa, Staphylococcus aureus [sequence omitted]

SEQ ID NO: 18 - DNA, 126 bp, Staphylococcus aureus
  source 1..126: mol_type = other DNA; note = S. aureus strain Mu50
atagagaatgccgataaagctatcaaggactttcaggacaacaaagcaccgcacgataagagcgctgcttacgaggccaattcaaaattgccaaaagacctccgagacaagaacaaccggttcgtt

SEQ ID NO: 19 - DNA, 174 bp, Staphylococcus aureus
  source 1..174: mol_type = other DNA; note = S. aureus strain Mu50
gagaaggttagtattgaaaaggccattgtgcgccatgacgaacgcgtcaagtctgccaacgatgctattagtaagctgaacgaaaaggattctatcgagaaccgaaggcttgctcaaagagaggttaacaaggcccctatggacgttaaagaacatttgcaaaagcaattggat

SEQ ID NO: 20 - protein, 15 aa, Severe acute respiratory syndrome-related coronavirus
  source 1..15: note = Severe acute respiratory syndrome coronavirus 2
MFVFLVLLPLVSSAA

SEQ ID NO: 21 - protein, 5 aa, synthetic construct
GGGGS

SEQ ID NO: 22 - protein, 5 aa, synthetic construct
  VARIANT 1..5: note = The entire sequence of amino acids 1-5 can be repeated one to ten times
GGGGS

SEQ ID NO: 23 - DNA, 125 bp, synthetic construct
  source 1..125: mol_type = other DNA
ttttttttttttttttttttttttttttttttttttttttttttttttttttttttttttttttttttttagtcatatgcttttttttttttttttttttttttttttttgctagctccagggtg

(19) United States
(12) Patent Application Publication
(10) Pub. No.: US 2025/0270299 A1
(43) Pub. Date: Aug. 28, 2025

(54) METHODS OF USING COMPOSITIONS COMPRISING SBI DOMAINS

(71) Applicant: Helix Nanotechnologies Inc, Boston, MA (US)

(72) Inventors: Hannu Rajaniemi, Corte Madera, CA (US); Nikolai Eroshenko, Boston, MA (US); Marianna Keaveney, Walpole, MA (US); Everett Webster, Boston, MA (US); Nikhil Dhar, Boston, MA (US); Arianna Lizeth Lopez Sauceda, Boston, MA (US); Katherine Rowe, Boston, MA (US); Justin Sean Huang, Jamaica Plain, MA (US); Dario De Jesus Davila Pasillas, Boston, MA (US); Taylor Gill, Cambridge, MA (US); Kemo Jammeh, Cambridge, MA (US); Justin Quinn, Malden, MA (US); Kyle Backman, Maynard, MA (US); Ravi Viswanath Kolla, Castro Valley, CA (US)

(21) Appl. No.: 19/041,656

(22) Filed: Jan. 30, 2025

Related U.S. Application Data
(60) Provisional application No. 63/627,352, filed on Jan. 31, 2024.

(51) Int. Cl. C07K 16/12; A61K 45/06; A61K 48/00; A61P 37/06; C12N 15/62

(52) U.S. Cl. CPC C07K 16/1271; A61K 48/005; C12N 15/62; C07K 2319/00

(57) ABSTRACT

Disclosed herein are fusion polypeptides comprising: (i) an antigen (e.g., an antigen fragment, an antigen variant or an antigen fragment variant); and (ii) a complement C3d-binding polypeptide from an immunoglobulin-binding protein (Sbi) of Staphylococcus aureus, and polynucleotides encoding the same. In some embodiments, the polynucleotide comprises: a modified nucleobase, a modified deoxyribose, a modified ribose, a modified backbone, or any combination thereof. Also disclosed herein are methods of using the fusion polypeptides and polynucleotides of the present disclosure, e.g., in immunosuppressed subjects.

### CROSS REFERENCE TO RELATED APPLICATIONS

This application claims priority to and the benefit of U.S. Provisional Patent Application No. 63/627,352 filed on Jan. 31, 2024, the entire contents of which are hereby incorporated by reference in its entirety.

### BACKGROUND

In recent years, progress has been made in the development of vaccines for a variety of diseases and conditions. However, these efforts have not been able to generate broadly neutralizing vaccines, particularly in immunosuppressed subjects.

### SUMMARY

This disclosure recognizes certain challenges in eliciting meaningful immune responses in immunosuppressed subjects, e.g., subjects who are receiving immunosuppressants, have a weakened immune system naturally or because of a disease, disorder, or condition, and/or are transplant recipients. For example, the present disclosure recognizes that immunosuppressed subjects often have the lowest vaccination responses (see Bin Lee et al., (2022) BMJ 2022; 376:e068632).

Among other things, the present disclosure provides certain insights and technologies for enhancing immunogenicity of an antigen in an immunosuppressed subject, thus allowing the subject to develop a meaningful immune response against the antigen. In some embodiments, a composition disclosed herein can be used to enhance immunogenicity of an antigen in an immunosuppressed subject. In some embodiments, administering an immunosuppressed subject with an antigen joined (e.g., directly or indirectly) to an Sbi protein as described herein (e.g., vaccinating) can enhance humoral responses and/or neutralizing titers in such subjects, as compared to administering with an antigen that is not joined (e.g., directly or indirectly) to an Sbi protein. In some embodiments, a composition disclosed herein comprise a fusion polypeptide, wherein the fusion polypeptide comprises (a) an antigen (e.g., as described herein); and (b) a complement C3d-binding polypeptide from an immunoglobulin-binding protein (Sbi) of Staphylococcus aureus. In some embodiments, an antigen comprises an epitope (e.g., a B cell and/or T cell epitope). In some embodiments, an antigen comprises an antigen variant, an antigen fragment, or an antigen fragment variant as described herein. In some embodiments, compositions disclosed herein comprise polynucleotides that encode a fusion polypeptide comprising (a) an antigen; and (b) a complement C3d-binding polypeptide from an immunoglobulin-binding protein (Sbi) of Staphylococcus aureus. In some embodiments, a polynucleotide encoding (a) and (b) comprises a modified nucleobase, a modified deoxyribose, a modified ribose, a modified backbone, or any combination thereof. In some embodiments, a composition disclosed herein (e.g., comprising fusion polypeptides or polynucleotides that encode a fusion polypeptide) can enhance titers of a resulting B cell response (e.g., including an antibody response) and/or T cell response. In some embodiments, a complement C3d-binding region of a Sbi protein from Staphylococcus aureus (e.g., Sbi III and/or Sbi IV) is an adjuvant. In some embodiments, a complement C3d-binding region of a Sbi protein from Staphylococcus aureus (e.g., Sbi III and/or Sbi IV) can modulate and/or enhance an activity of an immune cell (e.g., a B cell) expressing a complement receptor (e.g., CR2 or a variant or fragment thereof), e.g., by binding to C3d. Also provided herein are pharmaceutical compositions and methods of using said pharmaceutical compositions to stimulate an immune response against an antigen and/or to enhance immunogenicity of an antigen.

Among other things, this disclosure provides, a polynucleotide encoding a fusion polypeptide, wherein the fusion polypeptide comprises: (a) an antigen, and (b) a complement C3d-binding polypeptide from an immunoglobulin-binding protein (Sbi) of Staphylococcus aureus; wherein a polynucleotide comprises: a modified nucleobase, a modified deoxyribose, a modified ribose, a modified backbone, or any combination thereof.

In some embodiments, a polynucleotide is or comprises RNA.

In some embodiments, a polynucleotide is or comprises DNA.

In some embodiments, a complement C3d-binding polypeptide encoded by a polynucleotide disclosed herein is or comprises domain III of a Sbi of Staphylococcus aureus, or a functional fragment or a variant thereof. In some embodiments, a complement C3d-binding polypeptide encoded by a polynucleotide disclosed herein is or comprises domain IV of a Sbi of Staphylococcus aureus, or a functional fragment or a variant thereof. In some embodiments, a complement C3d-binding polypeptide encoded by a polynucleotide disclosed herein is or comprises one or both of domain III and domain IV of a Sbi of Staphylococcus aureus, or a functional fragment or a variant thereof.

In some embodiments, an antigen encoded by a polynucleotide disclosed herein or included in a fusion polypeptide disclosed herein is or comprises an antigen variant or antigen fragment variant that comprises at least one amino acid mutation (e.g., addition, deletion or substitution) compared to a target protein antigen.

In some embodiments, an antigen encoded by a polynucleotide disclosed herein or included in a fusion polypeptide disclosed herein comprises an antigen fragment, an antigen variant or an antigen fragment variant that is characterized in that, when expressed in vivo, the antigen fragment, antigen variant or antigen fragment variant binds to a Major Histocompatibility Complex (MHC) molecule (e.g., MHC class I and/or class II). In some embodiments, an antigen encoded by a polynucleotide disclosed herein or included in a fusion polypeptide disclosed herein comprises an antigen fragment, an antigen variant or an antigen fragment variant that is characterized in that, when expressed in vivo, the antigen fragment, antigen variant or antigen fragment variant comprises one or more B cell and/or T cell epitopes.

In some embodiments, an antigen encoded by a polynucleotide disclosed herein or included in a fusion polypeptide disclosed herein comprises an antigen fragment, an antigen variant or an antigen fragment variant that is characterized in that, when expressed in vivo, an antigen fragment, antigen variant or antigen fragment variant does not bind to an MHC molecule.

In some embodiments, one or more antigens encoded by a polynucleotide disclosed herein or included in a fusion polypeptide disclosed herein comprise one or more antigen fragments, one or more antigen variants or one or more antigen fragment variants which are or comprise one or more infectious disease antigens. In some embodiments, an infectious disease antigen comprises a viral antigen, a bacterial antigen, a fungal antigen, or combinations thereof. In some embodiments, one or more antigens encoded by a polynucleotide disclosed herein or included in a fusion polypeptide disclosed herein comprise one or more antigen fragments, one or more antigen variants or one or more antigen fragment variants which are or comprise one or more tumor and/or cancer antigens. In some embodiments, one or more antigens encoded by a polynucleotide disclosed herein or included in a fusion polypeptide disclosed herein comprise one or more antigen fragments, one or more antigen variants or one or more antigen fragment variants which are or comprise one or more neoantigens, one or more tumor associated antigens (TAAs) or one or more tumor specific antigens (TSAs).

In some embodiments, (a) is disposed N-terminus of (b). In some embodiments, (a) is disposed C-terminus of (b). In some embodiments, (a) and (b) are contiguous. In some embodiments, (a) and (b) are separated by a linker. In some embodiments: (a) is disposed N-terminus of (b), and (a) and (b) are contiguous. In some embodiments: (a) is disposed N-terminus of (b), and (a) and (b) are separated by a linker. In some embodiments: (a) is disposed C-terminus of (b), and (a) and (b) are contiguous. In some embodiments: (a) is disposed C-terminus of (b), and (a) and (b) are separated by a linker.

In some embodiments, a polynucleotide encodes a fusion polypeptide, wherein the fusion polypeptide comprises: (a) an antigen, (b) a complement C3d-binding polypeptide from an immunoglobulin-binding protein (Sbi) of Staphylococcus aureus, and (c) a linker. In some embodiments, a fusion polynucleotide comprises: a modified nucleobase, a modified deoxyribose, a modified ribose, a modified backbone, or any combination thereof.

In some embodiments, a linker is or comprises a peptidyl linker. In some embodiments, a linker is or comprises a peptidyl linker comprising at least 60% glycine and/or serine. In some embodiments, a linker is or comprises a Gly-Ser linker or a histidine linker.

In some embodiments, a linker is or comprises a Gly-Ser linker. In some embodiments, a Gly-Ser linker is or comprises a Gly-Gly-Gly-Gly-Ser (Gly4-Ser) linker.

In some embodiments, a linker is or comprises a histidine linker.

In some embodiments, a linker encoded by a polynucleotide comprises the sequence of SEQ ID NO: 6.

In some embodiments, a polynucleotide disclosed herein further comprises a nucleotide sequence encoding a secretion peptide. In some embodiments, a polynucleotide disclosed herein further comprises a nucleotide sequence encoding a polymerization domain. In some embodiments, a polynucleotide disclosed herein further comprises a nucleotide sequence encoding a trafficking domain. In some embodiments, a trafficking domain directs a polypeptide it is associated with to an MHC molecule (e.g., MHC class I and/or class II molecule). In some embodiments, a polynucleotide disclosed herein further comprises a nucleotide sequence encoding a transmembrane domain.

In some embodiments, a fusion polypeptide described herein further comprises a secretion peptide. In some embodiments, a fusion polypeptide described herein further comprises a polymerization domain. In some embodiments, a fusion polypeptide described herein further comprises a trafficking domain. In some embodiments, a trafficking domain directs a polypeptide it is associated with to an MHC molecule (e.g., MHC class I and/or class II molecule). In some embodiments, a fusion polypeptide described herein further comprises a transmembrane domain.

In some embodiments, a complement C3d-binding polypeptide, e.g., encoded by a polynucleotide disclosed herein, comprises: an Sbi domain III comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 3.

In some embodiments, a complement C3d-binding polypeptide, e.g., encoded by a polynucleotide disclosed herein, comprises: an Sbi domain IV comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 4.

In some embodiments, a complement C3d-binding polypeptide, e.g., encoded by a polynucleotide disclosed herein, comprises: (a) an Sbi domain III comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 3; (b) an Sbi domain IV comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 4; or (c) both (a) and (b).

In some embodiments, a complement C3d-binding polypeptide encoded by a polynucleotide disclosed herein comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 5.

In some embodiments, a polynucleotide disclosed herein encodes a fusion polypeptide having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 8.

In some embodiments, a polynucleotide disclosed herein has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 10.

In some embodiments, a polynucleotide disclosed herein has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 18.

In some embodiments, a polynucleotide disclosed herein has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 19.

In some embodiments, a polynucleotide is a polyribonucleotide comprising one or more modified ribonucleotides comprising: a modified nucleobase, a modified ribose, a modified backbone, or any combination thereof.

In some embodiments, one or more modified ribonucleotides has: a 5′ monophosphate; a 5′ diphosphate; or a 5′ triphosphate. In some embodiments, one or more modified ribonucleotides comprises a 5′ triphosphate.

In some embodiments, one or more modified ribonucleotides comprises a nucleoside comprising an acetyl group, wherein the nucleoside is N4-acetylcytidine. In some embodiments, one or more modified ribonucleotides has a structure provided in Table 3A. In some embodiments, one or more modified ribonucleotides has a structure provided in Table 3B when incorporated in a polynucleotide (e.g., polyribonucleotide). In some embodiments, a polynucleotide (e.g., polyribonucleotide) comprises one or more modified ribonucleotides that have a structure provided in Table 3B.

In some embodiments, the nucleoside has a structure of:

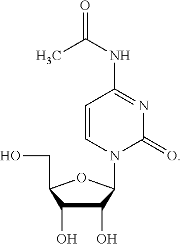

In some embodiments, one or more modified ribonucleotides has the structure of:

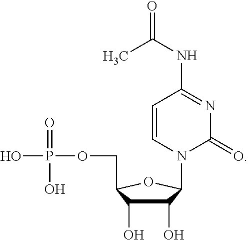

In some embodiments, one or more modified ribonucleotides has the structure of:

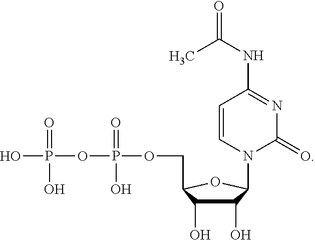

In some embodiments, one or more modified ribonucleotides has the structure of:

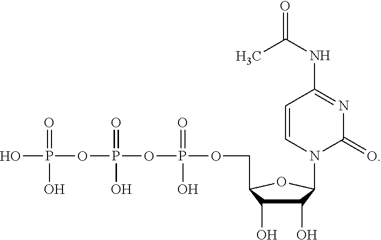

In some embodiments, a polynucleotide (e.g., polyribonucleotide) comprises one or more modified ribonucleotides that have the following structure:

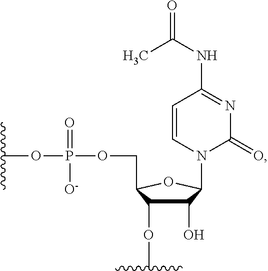

wherein [symbol] indicates the position of attachment to an adjacent ribonucleotide.

In some embodiments, a polynucleotide (e.g., polyribonucleotide) comprises one or more modified ribonucleotides that have the following structure:

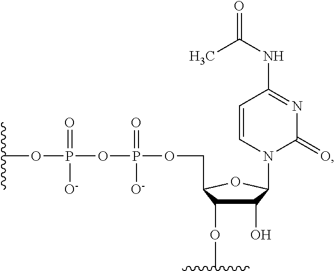

wherein [symbol] indicates the position of attachment to an adjacent ribonucleotide.

In some embodiments, a polynucleotide (e.g., polyribonucleotide) comprises one or more modified ribonucleotides that have the following structure:

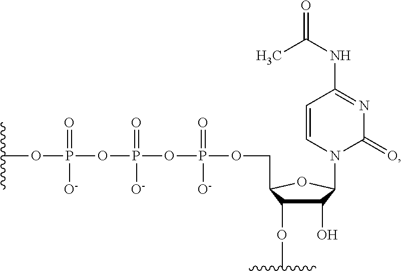

wherein [symbol] indicates the position of attachment to an adjacent ribonucleotide.

In some embodiments, a polyribonucleotide comprises cytidine nucleosides, and at least 5% of cytidine nucleosides in a polyribonucleotide comprise N4-acetylcytidine.

In some embodiments, a polyribonucleotide comprises cytidine nucleosides, and less than 100% of cytidine nucleosides in the polyribonucleotide comprise N4-acetylcytidine.

In some embodiments, a polyribonucleotide comprises cytidine nucleosides, and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of cytidine nucleosides in a polyribonucleotide comprise N4-acetylcytidine.

In some embodiments, one or more modified ribonucleotides comprises a nucleoside comprising a hydroxymethyl group, wherein the nucleoside is 5-hydroxymethyluridine. In some embodiments, one or more modified ribonucleotides has a structure provided in Table 3A. In some embodiments, one or more modified ribonucleotides has a structure provided in Table 3B when incorporated in a polynucleotide (e.g., polyribonucleotide). In some embodiments, a polynucleotide (e.g., polyribonucleotide) comprises one or more modified ribonucleotides that have a structure provided in Table 3B.

In some embodiments, the nucleoside has a structure of:

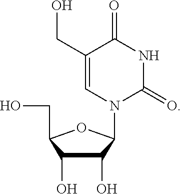

In some embodiments, one or more modified ribonucleotides has a structure of:

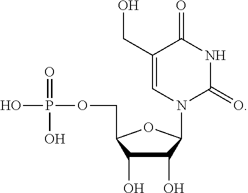

In some embodiments, one or more modified ribonucleotides has a structure of:

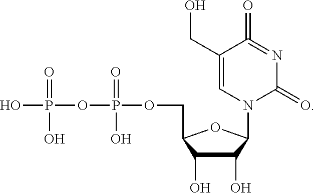

In some embodiments, one or more modified ribonucleotides has a structure of:

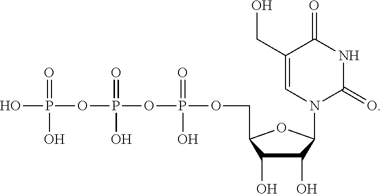

In some embodiments, a polynucleotide (e.g., polyribonucleotide) comprises one or more modified ribonucleotides that have the following structure:

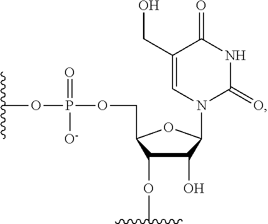

wherein [symbol] indicates the position of attachment to an adjacent ribonucleotide.

In some embodiments, a polynucleotide (e.g., polyribonucleotide) comprises one or more modified ribonucleotides that have the following structure:

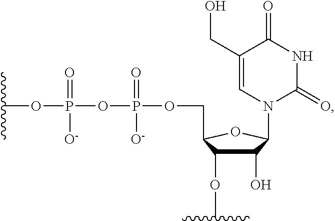

wherein [symbol] indicates the position of attachment to an adjacent ribonucleotide.

In some embodiments, a polynucleotide (e.g., polyribonucleotide) comprises one or more modified ribonucleotides that have the following structure:

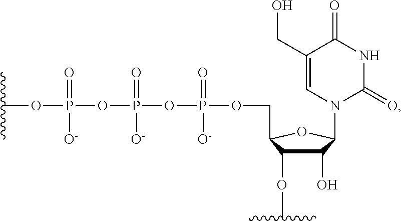

wherein [symbol] indicates the position of attachment to an adjacent ribonucleotide.

In some embodiments, a polyribonucleotide comprises uridine nucleosides and at least 5% of uridine nucleosides in the polyribonucleotide comprise 5-hydroxymethyluridine.

In some embodiments, a polyribonucleotide comprises uridine nucleosides and less than 100% of uridine nucleosides in the polyribonucleotide comprise 5-hydroxymethyluridine.

In some embodiments, a polyribonucleotide comprises uridine nucleosides and at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of uridine nucleosides in a polyribonucleotide comprise 5-hydroxymethyluridine.

In some embodiments, a polyribonucleotide comprises uridine nucleosides and more than 60% of uridine nucleosides in a polyribonucleotide comprise 5-hydroxymethyluridine.

In some embodiments, one or more modified ribonucleotides comprises: N4-acetylcytidine, 5-hydroxymethyluridine, N1-methylpseudouridine, pyridin-4-one ribonucleoside, 5-aza-uridine, 6-aza-uridine, 2-thio-5-aza-uridine, 2-thio-uridine (s2U), 5-methyl cytidine (m5C), 5-aza-cytidine, 6-aza-cytidine, pseudoisocytidine, 3-methyl-cytidine (m3C), 5-formyl-cytidine (f5C), N4-methyl-cytidine (m4C), 2-amino-purine, 2, 6-diaminopurine, 2-amino-6-halo-purine, 6-halo-purine, inosine (I), 1-methyl-inosine (ml I), wyosine (imG), methylwyosine (mimG), 5-hydroxycytidine, 5-hydroxymethylcytidine, 5-carboxycytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N1-hydroxypseudouridine, 2′-O-methyl-N1-methylpseudouridine, N1-ethylpseudouridine, N1-hydroxymethylpseudouridine, ara-uridine, N6-methyladenosine, 2-aminoadenosine, 3-methyladenosine, 7-deazaadenosine, 8-oxoadenosine, thienoguanosine, 7-deazaguanosine, 8-oxoguanosine, 6-O-methylguanine, or any combination thereof. In some embodiments, one or more modified ribonucleotides comprises a nucleoside comprising a ribose moiety comprising an acetyl group, wherein the ribose is 2′-O-acetylated.

In some embodiments, one or more modified ribonucleotide has a structure of:

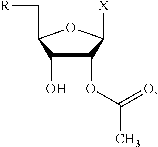

(a) wherein R is a monophosphate, a diphosphate, or a triphosphate; and
(b) wherein X is an adenine nucleobase, a guanine nucleobase, a cytosine nucleobase (e.g., N4-acetylcytosine), or a uracil nucleobase (e.g., 5-hydroxymethyluracil).

In some embodiments, one or more modified ribonucleotides comprises: (i) a 2′-O-acetylated ribose and (ii) an adenine nucleobase. In some embodiments, one or more modified ribonucleotides has a structure provided in Table 3C. In some embodiments, one or more modified ribonucleotides has a structure provided in Table 3D when incorporated in a polynucleotide. In some embodiments, a polynucleotide (e.g., a polyribonucleotide) comprises one or more modified ribonucleotides that have a structure provided in Table 3D.

In some embodiments, a polynucleotide (e.g., polyribonucleotide) comprises one or more modified ribonucleotides that have the following structure:

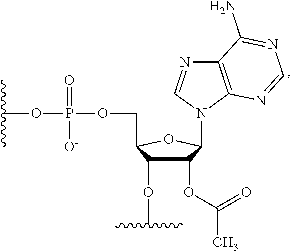

wherein [symbol] indicates the position of attachment to an adjacent ribonucleotide.

In some embodiments, a polynucleotide (e.g., polyribonucleotide) comprises one or more modified ribonucleotides that have the following structure:

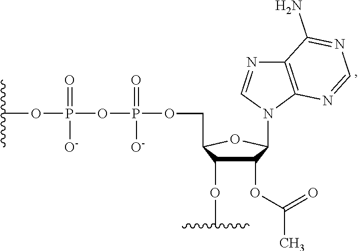

wherein [symbol] indicates the position of attachment to an adjacent ribonucleotide.

In some embodiments, a polynucleotide (e.g., polyribonucleotide) comprises one or more modified ribonucleotides that have the following structure:

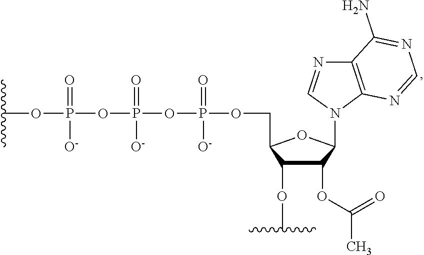

wherein [symbol] indicates the position of attachment to an adjacent ribonucleotide.

In some embodiments, one or more modified ribonucleotides comprises: (i) a 2′-O-acetylated ribose and (ii) a guanine nucleobase. In some embodiments, one or more modified ribonucleotides has a structure provided in Table 3C. In some embodiments, one or more modified ribonucleotides has a structure provided in Table 3D when incorporated in a polynucleotide. In some embodiments, a polynucleotide (e.g., a polyribonucleotide) comprises one or more modified ribonucleotides that have a structure provided in Table 3D.

In some embodiments, a polynucleotide (e.g., polyribonucleotide) comprises one or more modified ribonucleotides that have the following structure:

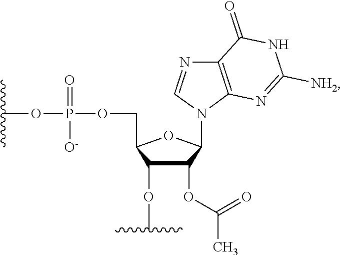

wherein [symbol] indicates the position of attachment to an adjacent ribonucleotide.

In some embodiments, a polynucleotide (e.g., polyribonucleotide) comprises one or more modified ribonucleotides that have the following structure:

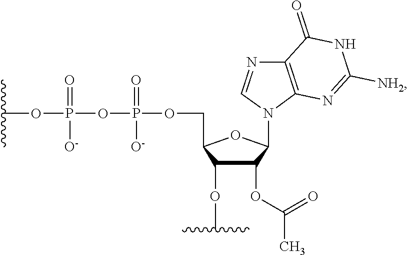

wherein [symbol] indicates the position of attachment to an adjacent ribonucleotide.

In some embodiments, a polynucleotide (e.g., polyribonucleotide) comprises one or more modified ribonucleotides that have the following structure:

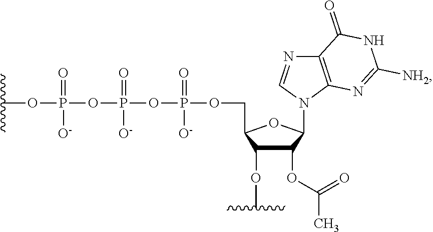

wherein [symbol] indicates the position of attachment to an adjacent ribonucleotide.

In some embodiments, one or more modified ribonucleotides comprises: (i) a 2′-O-acetylated ribose and (ii) a cytosine nucleobase (e.g., cytosine or N4-acetylcytosine). In some embodiments, one or more modified ribonucleotides has a structure provided in Table 3C. In some embodiments, one or more modified ribonucleotides has a structure provided in Table 3D when incorporated in a polynucleotide. In some embodiments, a polynucleotide (e.g., a polyribonucleotide) comprises one or more modified ribonucleotides that have a structure provided in Table 3D.

In some embodiments, a polynucleotide (e.g., polyribonucleotide) comprises one or more modified ribonucleotides that have the following structure:

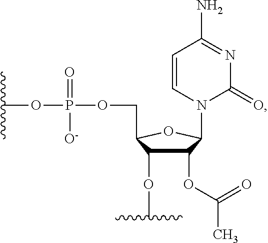

wherein [symbol] indicates the position of attachment to an adjacent ribonucleotide.

In some embodiments, a polynucleotide (e.g., polyribonucleotide) comprises one or more modified ribonucleotides that have the following structure:

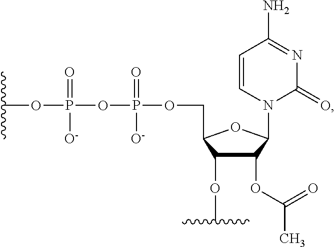

wherein [symbol] indicates the position of attachment to an adjacent ribonucleotide.

In some embodiments, a polynucleotide (e.g., polyribonucleotide) comprises one or more modified ribonucleotides that have the following structure:

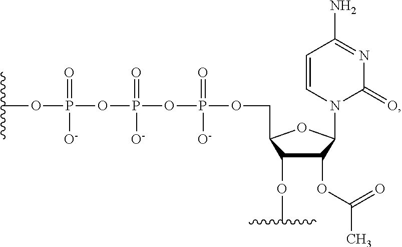

wherein [symbol] indicates the position of attachment to an adjacent ribonucleotide.

In some embodiments, one or more modified ribonucleotides comprising a 2′-O-acetylated ribose comprises a N4-acetylcytosine nucleobase. In some embodiments, one or more modified ribonucleotides has a structure provided in Table 3C. In some embodiments, one or more modified ribonucleotides has a structure provided in Table 3D when incorporated in a polynucleotide. In some embodiments, a polynucleotide (e.g., a polyribonucleotide) comprises one or more modified ribonucleotides that have a structure provided in Table 3D.

In some embodiments, a polynucleotide (e.g., polyribonucleotide) comprises one or more modified ribonucleotides that have the following structure:

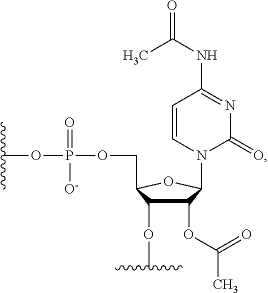

wherein [symbol] indicates the position of attachment to an adjacent ribonucleotide.

In some embodiments, a polynucleotide (e.g., polyribonucleotide) comprises one or more modified ribonucleotides that have the following structure:

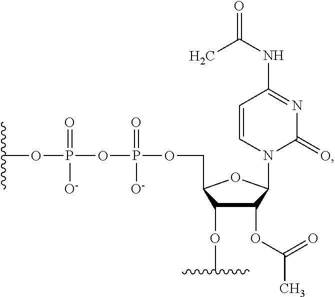

wherein [symbol] indicates the position of attachment to an adjacent ribonucleotide.

In some embodiments, a polynucleotide (e.g., polyribonucleotide) comprises one or more modified ribonucleotides that have the following structure:

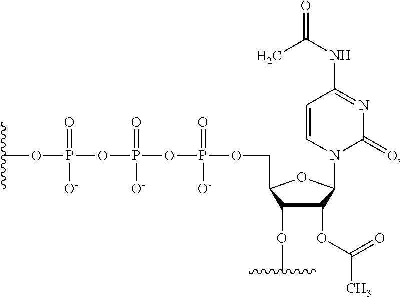

wherein [symbol] indicates the position of attachment to an adjacent ribonucleotide.

In some embodiments, one or more modified ribonucleotides comprising a 2′-O-acetylated ribose comprises a uracil nucleobase (e.g., uracil or 5-hydroxymethyluracil). In some embodiments, one or more modified ribonucleotides has a structure provided in Table 3C. In some embodiments, one or more modified ribonucleotides has a structure provided in Table 3D when incorporated in a polynucleotide. In some embodiments, a polynucleotide (e.g., a polyribonucleotide) comprises one or more modified ribonucleotides that have a structure provided in Table 3D.

In some embodiments, a polynucleotide (e.g., polyribonucleotide) comprises one or more modified ribonucleotides that have the following structure:

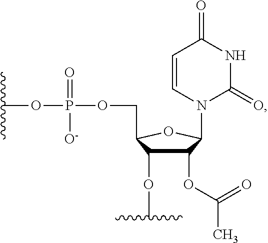

wherein [symbol] indicates the position of attachment to an adjacent ribonucleotide.

In some embodiments, a polynucleotide (e.g., polyribonucleotide) comprises one or more modified ribonucleotides that have the following structure:

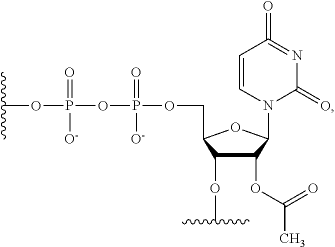

wherein [symbol] indicates the position of attachment to an adjacent ribonucleotide.

In some embodiments, a polynucleotide (e.g., polyribonucleotide) comprises one or more modified ribonucleotides that have the following structure:

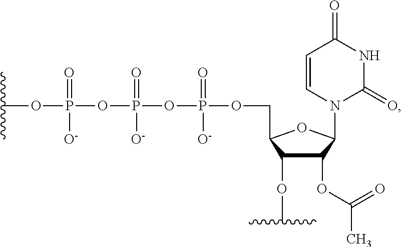

wherein [symbol] indicates the position of attachment to an adjacent ribonucleotide.

In some embodiments, one or more modified ribonucleotides comprising a 2′-O-acetylated ribose comprises a 5-hydroxymethyluracil nucleobase. In some embodiments, one or more modified ribonucleotides has a structure provided in Table 3C. In some embodiments, one or more modified ribonucleotides has a structure provided in Table 3D when incorporated in a polynucleotide. In some embodiments, a polynucleotide (e.g., a polyribonucleotide) comprises one or more modified ribonucleotides that have a structure provided in Table 3D.

In some embodiments, a polynucleotide (e.g., polyribonucleotide) comprises one or more modified ribonucleotides that have the following structure:

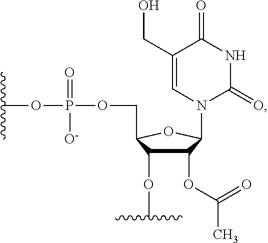

wherein [symbol] indicates the position of attachment to an adjacent ribonucleotide.

In some embodiments, a polynucleotide (e.g., polyribonucleotide) comprises one or more modified ribonucleotides that have the following structure:

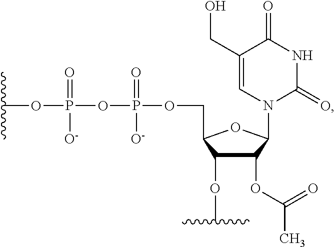

wherein [symbol] indicates the position of attachment to an adjacent ribonucleotide.

In some embodiments, a polynucleotide (e.g., polyribonucleotide) comprises one or more modified ribonucleotides that have the following structure:

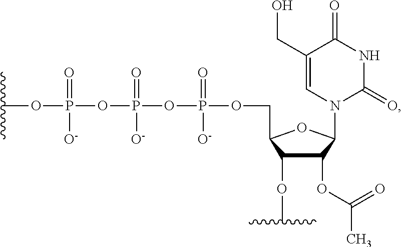

wherein [symbol] indicates the position of attachment to an adjacent ribonucleotide.

In some embodiments, one or more modified ribonucleotides comprising a 2′-O-acetylated ribose comprises a N1-methylpseudouracil nucleobase. In some embodiments, one or more modified ribonucleotides has a structure provided in Table 3C. In some embodiments, one or more modified ribonucleotides has a structure provided in Table 3D when incorporated in a polynucleotide. In some embodiments, a polynucleotide (e.g., a polyribonucleotide) comprises one or more modified ribonucleotides that have a structure provided in Table 3D.

In some embodiments, a polynucleotide (e.g., polyribonucleotide) comprises one or more modified ribonucleotides that have the following structure:

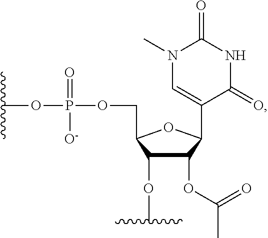

wherein [symbol] indicates the position of attachment to an adjacent ribonucleotide.

In some embodiments, a polynucleotide (e.g., polyribonucleotide) comprises one or more modified ribonucleotides that have the following structure:

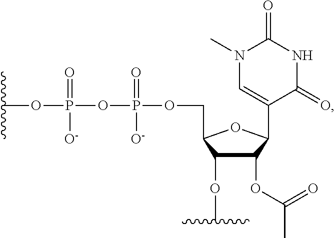

wherein [symbol] indicates the position of attachment to an adjacent ribonucleotide.

In some embodiments, a polynucleotide (e.g., polyribonucleotide) comprises one or more modified ribonucleotides that have the following structure:

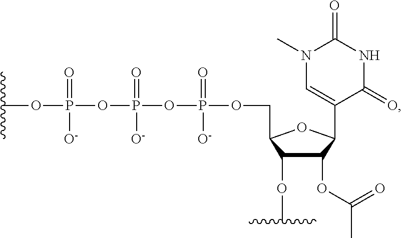

wherein [symbol] indicates the position of attachment to an adjacent ribonucleotide.

In some embodiments of any of the polyribonucleotides disclosed herein, at least 5% of ribose moieties are acetylated (2′-O-acetylated).

In some embodiments of any of the polyribonucleotides disclosed herein, about 5% to about 99% of ribose moieties are acetylated (2′-O-acetylated).

In some embodiments of any of the polyribonucleotides disclosed herein, a polyribonucleotide comprises a cap structure and a cap structure does not comprise a 2′-O-acetylated ribose.

In some embodiments of any of the polyribonucleotides disclosed herein, a polyribonucleotide comprises a cap structure and a cap structure comprises a 2′-O-acetylated ribose.

In some embodiments of any of the polyribonucleotides disclosed herein, a polyribonucleotide further comprises one or more ribonucleotides that does not comprise a 2′-0 acetylated ribose.

In some embodiments of any of the polyribonucleotides disclosed herein, a polyribonucleotide comprises a coding region. In some embodiments, a polyribonucleotide is or comprises an RNA oligo; a messenger RNA (mRNA); a gRNA; an inhibitory RNA; an miRNA or siRNA; an antisense oligonucleotide; or any combination thereof.

This disclosure further provides a fusion polypeptide encoded by a polynucleotide of disclosed herein.

In some embodiments, a fusion polypeptide has at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 8.

In some embodiments of a fusion polypeptide disclosed herein, a complement C3d-binding polypeptide comprises an Sbi domain III comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 3. In some embodiments of a fusion polypeptide disclosed herein, a complement C3d-binding polypeptide comprises an Sbi domain IV comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 4. In some embodiments of a fusion polypeptide disclosed herein, a complement C3d-binding polypeptide comprises: (a) an Sbi domain III comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 3; or (b) an Sbi domain IV comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 4; or (c) both (a) and (b).

In some embodiments of a fusion polypeptide disclosed herein, a complement C3d-binding polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 5.

In some embodiments of a fusion polypeptide disclosed herein, a polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 8.

Also disclosed herein is an expression vector comprising a polynucleotide disclosed herein. In some embodiments, an expression vector comprises a viral vector that is a retrovirus vector, an adenovirus vector, an adeno-associated virus vector or a lentivirus vector or an RNA vector.

This disclosure further provides, a composition for delivering a polyribonucleotide disclosed herein, a fusion polypeptide disclosed herein, or an expression vector disclosed herein.

In some embodiments, a composition is a pharmaceutical composition. In some embodiments, a pharmaceutical composition is or comprises an immunogenic composition, a vaccine, a gene therapy, a chemotherapy, a protein replacement therapy, an immunotherapy, an antibody therapy, an immune-modulation therapy, a cell engineering therapy, or a combination thereof.

Also provided herein is a cell comprising a polyribonucleotide disclosed herein, a fusion polypeptide disclosed herein, or an expression vector disclosed herein.

Further provided herein is a method of making a polynucleotide comprising: recombinantly joining (i) a first nucleotide sequence that encodes an antigen, e.g., an antigen fragment, comprising an epitope of a target protein antigen, and (ii) a second nucleotide sequence that encodes a complement C3d-binding polypeptide from an immunoglobulin-binding protein (Sbi) of Staphylococcus aureus, wherein the joining forms a polynucleotide comprising the first nucleotide sequence and the second nucleotide sequence. In some embodiments, a first nucleotide sequence and a second nucleotide sequence are joined directly to one another. In some embodiments, a first nucleotide sequence and a second nucleotide sequence are indirectly joined (e.g., with a nucleotide encoding a linker between the first and second nucleotide sequences).

Among other things, this disclosure provides a method comprising administering to a subject in need thereof at least one dose of a pharmaceutical composition disclosed herein. In some embodiments, at least one dose is administered in an effective amount to: (i) induce an immune response against the antigen in a subject; (ii) stimulate B cells in a subject; or (iii) both (i) and (ii).

In some embodiments, an immunosuppressed subject does not generate a functional T cell response.

In some embodiments, B cells are stimulated in a subject without inducing a T cell response.

In some embodiments, B cells are stimulated in a subject in the absence of functional T cells (e.g., no T cells or no T cell function).

In some embodiments, B cells are stimulated in a subject in the presence of T cells. In some embodiments, T cells in a subject have a reduced ability to induce an immune response.

Provided herein is a method for enhancing an immunogenicity of an antigen, comprising administering to an immunosuppressed subject, a polynucleotide disclosed herein, or a pharmaceutical composition disclosed herein.

In some embodiments, enhancing immunogenicity of an antigen comprises stimulating an immune response against an antigen. In some embodiments, enhancing immunogenicity of an antigen comprises stimulating a humoral immune response. In some embodiments, a humoral immune response is an antibody response.

This disclosure provides, a method for stimulating an immune response against an antigen, comprising administering to an immunosuppressed subject, e.g., an immunocompromised subject, a polynucleotide disclosed herein, or a pharmaceutical composition disclosed herein.

In some embodiments, an immunosuppressed subject has or has been diagnosed with a rare disease, a lung disease, a liver disease, a kidney disease, a blood disorder, an autoimmune disease, an immunodeficiency disorder, a cardiovascular disorder, a cancer, or any combination thereof.

In some embodiments, an immunosuppressed subject has received, is receiving, or will be receiving one or more transplants. In some embodiments, an immunosuppressed subject who has received, is receiving, or will be receiving one or more transplants can also be administered one or more therapeutic agents and/or be subjected to one or more procedures, e.g., dialysis. In some embodiments, an immunosuppressed subject has received, is receiving, or will be receiving one or more dialysis treatments. In some embodiments, a dialysis treatment is or comprises hemodialysis. In some embodiments, a dialysis treatment is or comprises peritoneal dialysis.

In some embodiments, an immunosuppressed subject has received, is receiving, or will be receiving one or more dialysis treatments. In some embodiments, a dialysis treatment is or comprises hemodialysis. In some embodiments, a dialysis treatment is or comprises peritoneal dialysis.

In some embodiments, an immunosuppressed subject has received or is receiving an immunosuppressive therapy. In some embodiments, an immunosuppressive therapy comprises: an organ transplant conditioning regimen, a therapy that suppresses an immune system (e.g., an antibody therapy), a B-cell targeting therapy, a T-cell targeting therapy, a chemotherapy, a radiation therapy, a cancer therapy, a treatment for an inflammatory disease and/or a treatment for an autoimmune disease.

In some embodiments, one or more transplants is an organ transplant. In some embodiments, an organ transplant comprises: a kidney transplant, a liver transplant, a heart transplant, a lung transplant, a pancreas transplant, a stomach transplant, an intestine transplant, or any combination thereof.

In some embodiments, one or more transplants is a cell transplant. In some embodiments, a cell transplant is a transplant of a population of stem cells (e.g., hematopoietic stem cells, induced pluripotent stem cells, or embryonic stem cells), immune cells (e.g., T cells or macrophages), or any combination thereof. In some embodiments, a cell transplant is a transplant of a population of bone marrow cells, blood cells, or any combination thereof. In some embodiments, a cell transplant is a transplant of a population of engineered cells. In some embodiments, a cell transplant is a transplant of a population of non-engineered cells.

In some embodiments, one or more transplants is a tissue transplant. In some embodiments, a tissue transplant comprises skin tissue transplant, bone tissue transplant, cartilage tissue transplant, adrenal tissue transplant, corneal tissue transplant, or any combination thereof.

In some embodiments, a transplant is an allogeneic transplant. In some embodiments, a transplant is an autologous transplant.

In some embodiments of any of the methods disclosed herein, a method stimulates a humoral immune response. In some embodiments, a humoral response is an antibody response.

In some embodiments of any of the methods disclosed herein, administration of a pharmaceutical composition or a polynucleotide disclosed herein, results in an increased antibody response (e.g., an increased titer or an increased concentration of an antibody response). In some embodiments, an increased titer of an antibody response is compared to administration of an otherwise similar composition that does not comprise a polynucleotide comprising an Sbi domain III, or a fragment or variant thereof; and/or an Sbi domain IV, or a fragment or a variant thereof.

In some embodiments, an antibody response (e.g., a titer or a concentration of an antibody response) is increased by about 1.5-fold to about 500-fold, about 1.5-fold to about 400-fold, about 1.5-fold to about 300-fold, about 1.5-fold to about 200-fold, about 1.5-fold to about 100-fold, about 1.5-fold to about 50-fold, about 1.5-fold to about 20-fold, about 1.5-fold to about 10-fold, about 1.5-fold to about 5-fold, about 1.5-fold to about 2 fold, about 2-fold to about 500-fold, about 5-fold to about 500-fold, about 10-fold to about 500-fold, about 20-fold to about 500-fold, about 50-fold to about 500-fold, about 100-fold to about 500-fold, about 200-fold to about 500-fold, about 300-fold to about 500-fold, or about 400-fold to about 500-fold.

In some embodiments, an antibody response is increased by about 1.5-fold, about 2-fold, about 5-fold, about 10-fold, about 20-fold, about 50-fold, about 100-fold, about 200-fold, about 300-fold, about 400-fold, or about 500-fold.

In some embodiments, an antibody response is increased by at least 1.5-fold, at least 2-fold, at least 5-fold, at least 10-fold, at least 20-fold, at least 50-fold, at least 100-fold, at least 200-fold, at least 300-fold, at least 400-fold, or at least 500-fold.

In some embodiments of any of the methods disclosed herein, an immunosuppressed subject has received or is receiving an immunosuppressive therapy. In some embodiments, an immunosuppressive therapy comprises: an organ transplant conditioning regimen, chemotherapy, radiation therapy, or a treatment for a cancer, an inflammatory disease and/or an autoimmune disease.

In some embodiments, an organ transplant conditioning regimen comprises a calcineurin inhibitor, an antiproliferative agent, a steroid, an mTOR inhibitor, or any combination thereof. In some embodiments, a calcineurin inhibitor comprises tacrolimus. In some embodiments, an antiproliferative agent comprises mycophenolate mofetil. In some embodiments, a steroid comprises prednisone.

In some embodiments, an organ transplant conditioning regimen, e.g., an immunosuppression induction and/or maintenance treatment regimen, comprises tacrolimus, mycophenolate mofetil, prednisone, or any combination thereof.

Also provided herein is a composition comprising a polynucleotide disclosed herein, or a pharmaceutical composition of disclosed herein, for use in: enhancing an immunogenicity of an antigen, or for stimulating an immune response against an antigen.

Further provided herein is the use of a composition comprising a polynucleotide disclosed herein, or a pharmaceutical composition disclosed herein, in the preparation of a medicament for enhancing the immunogenicity of an antigen, or for stimulating an immune response against an antigen.

In some embodiments of any one of the uses, or compositions for use disclosed herein, a polynucleotide or pharmaceutical composition is administered to a subject.

In some embodiments of any one of the uses, or compositions for use disclosed herein, a subject is an immunosuppressed subject, e.g., an immunocompromised subject. In some embodiments, an immunosuppressed subject has received, is receiving, or will be receiving one or more transplants. In some embodiments, an immunosuppressed subject who has received, is receiving, or will be receiving one or more transplants can also be administered one or more therapeutic agents and/or be subjected to one or more procedures, e.g., dialysis. In some embodiments, an immunosuppressed subject has received, is receiving, or will be receiving one or more dialysis treatments. In some embodiments, a dialysis treatment is or comprises hemodialysis. In some embodiments, a dialysis treatment is or comprises peritoneal dialysis.

In some embodiments, one or more transplants is an organ transplant. In some embodiments, an organ transplant comprises: a kidney transplant, a liver transplant, a heart transplant, a lung transplant, a pancreas transplant, a stomach transplant, an intestine transplant, or any combination thereof.

In some embodiments, one or more transplants is a cell transplant. In some embodiments, a cell transplant is a transplant of a population of stem cells (e.g., hematopoietic stem cells, induced pluripotent stem cells, or embryonic stem cells), immune cells, or any combination thereof. In some embodiments, a cell transplant is a transplant of a population of bone marrow cells, blood cells, or any combination thereof. In some embodiments, a cell transplant is a transplant of a population of engineered cells. In some embodiments, a cell transplant is a transplant of a population of non-engineered cells.

In some embodiments, one or more transplants is a tissue transplant. In some embodiments, a tissue transplant comprises skin tissue transplant, bone tissue transplant, cartilage tissue transplant, adrenal tissue transplant, corneal tissue transplant, or any combination thereof.

In some embodiments of any of the methods, uses, or compositions for use disclosed herein, a transplant is an allogeneic transplant.

In some embodiments of any of the methods, uses, or compositions for use disclosed herein, a single dose, or a plurality of doses of a polynucleotide or pharmaceutical composition is administered to a subject. In some embodiments, a first dose and one or more subsequent doses are in the same amount amounts. In some embodiments, a first dose and one or more subsequent doses are in different amount amounts. In sone embodiments, a first dose and one or more subsequent doses are administered by at least 3 days, at least 4 days, at least 5 days, at least 6 days, at least 7 days, at least 2 weeks, at least 3 weeks, or at least 4 weeks apart.

In some embodiments of any of the methods, uses, or compositions for use disclosed herein, a pharmaceutical composition is administered in combination with one or more therapeutic compositions, e.g., standard of care.

In some embodiments of any of the methods, uses, or compositions for use disclosed herein, a subject is a mammal.

In some embodiments of any of the methods, uses, or compositions for use disclosed herein, a subject is a human.

These, and other aspects encompassed by the present disclosure, are described in more detail below and in the claims.

### BRIEF DESCRIPTION OF THE DRAWINGS

FIGS. 1A-1D demonstrate improved immunogenicity of exemplary fusion polypeptides that comprise an isolated SARS-CoV-2 S protein domain with the third and fourth domains of Sbi (“Sbi(III-IV)”). FIG. 1A is a schematic showing a hypothesized mechanism of action of fusion polypeptides comprising Sbi(III-IV). Without wishing to be bound to any particular theory, Sbi(III-IV) becomes coated in the degradation products of C3 complement proteins including C3d, which in turn can drive B cell activation by binding to CR2. C3d-driven B cell co-stimulation is orthogonal to helper T cell activation, which canonically occurs through TCR recognition of a cognate MHCII-peptide complex and CD40 signaling. FIG. 1B is a bar graph showing an exemplary fusion polypeptide comprising a Receptor Binding Domain of SARS-CoV-2 S protein (referred to herein as “RBD”) and an Sbi(III-IV) led to increased IgG titers in BALB/c mice at Day 21 following a 10 μg dose of IM-delivered RNA. Co-delivering an RBD joined (e.g., directly or indirectly) to the transmembrane domain of S protein (at total RNA dose of 10 μg) in addition to the exemplary fusion polypeptide leads to a further increase in titer FIG. 1C is a bar graph with ELISPOT counts of the number of IFNγ/IL2 double-stained colonies. For each condition, spleens from 5 mice were pooled and plated to 4 wells at 500,000 cells/well. 2 wells each were stimulated by either N-terminal or C-terminal S protein peptide pools (PepMix SARS-CoV-2 Spike Glycoprotein mix). Rates were calculated by pooling colony counts across the 4 wells, and the error bars were calculated as the Poisson error of the pooled colony counts. FIG. 1D is a bar graph depicting exemplary fusion polypeptides comprising fragments of an RBD joined (e.g., directly or indirectly) to Sbi(III-IV), which following administration, led to detectable humoral responses.

FIGS. 2A-2B depict a high-throughput protein folding screen. FIG. 2A is a schematic of a screen for protein folding. Anti-sera are generated by vaccinating mice with an RNA vaccine encoding a full-length protein (e.g., a control or an RBD) of interest. The collected anti-sera are then used to detect properly folded fragments presented in the context of a mammalian display system in a library of protein fragments. FIG. 2B is a bar graph showing positive and negative controls to validate the screening strategy. RBD was presented on the surface of HEK 293T cells and stained with anti-sera collected from RBD-vaccinated mice.

FIG. 3 is a bar graph showing increased virus neutralization with an exemplary fusion polypeptide comprising an Sbi(III-IV) joined (e.g., directly or indirectly) to an RBD.

FIGS. 4A-4B are bar graphs showing stimulation of B and T cells by an exemplary fusion polypeptide comprising an Sbi(III-IV) joined (e.g., directly or indirectly) to an RBD. The top and bottom horizontal lines indicate the detection limit of the dilution series used in the titer measurement. Sera that had titers that fell outside of that range were given values from the extreme ends of the dilution series. Values were calculated as geometric means, with the error bars corresponding to geometric standard deviation.

FIGS. 5A-5B are bar graphs showing the results of a vaccination regimen comprising a priming dose and a booster dose. The top and bottom horizontal lines indicate the detection limit of the dilution series used in the titer measurement. Sera that had titers that fell outside of that range were given values from the extreme ends of the dilution series. Values were calculated as geometric means, with the error bars corresponding to geometric standard deviation.

FIG. 6 is a bar graph showing antibody titer generated with a vaccination regimen comprising a priming dose and a booster dose. The top and bottom horizontal lines indicate the detection limit of the dilution series used in the titer measurement. Sera that had titers that fell outside of that range were given values from the extreme ends of the dilution series. Values were calculated as geometric means, with the error bars corresponding to geometric standard deviation.

FIGS. 7A-7D are graphs showing body weights of female mice (FIGS. 7A and 7C) and male mice (FIGS. 7B and 7D) treated with Tacrolimus, Mycophenolate Mofetil and Prednisone (referred to herein as “TMP”) or saline, with or without vaccination. Bars represent mean+standard error of the mean (SEM).

FIGS. 8A-8B are graphs of body temperatures of female mice (FIG. 8A) and male mice (FIG. 8B) treated with TMP or saline, with or without vaccination.

FIGS. 9A-9F are bar graphs showing certain immune-cell counts on Day 3 in female and male mice treated with TMP or saline. Bars represent mean+SD. In each graph there is a set of two bars for each condition (TMP or saline) with the first bar representing data from female mice and the second bar representing data from male mice. For example, in FIG. 9A, the graph has a set of two bars for TMP and a set of two bars for saline. In each of these sets of bars, the first bar is data from female mice and the second bar is data from male mice.

FIGS. 10A-10D are bar graphs of cell cycle phases of CD4+T and CD8+ T cell phases on day of priming (Day 3) in female (FIGS. 10A and 10C) or male (FIGS. 10B and 10D) mice treated with TMP or saline. ****P<0.0001 by 2-way ANOVA with Sidak's post hoc test compared to saline. Bars represent mean+SD. In each graph, for each condition (TMP or saline) there is a set of four bars representing phases of the cell cycle, in the following order: G0, G1, S and M.

FIGS. 11A-11F are bar graphs of certain immune cell-counts on Day 17 of female and male mice treated with TMP or saline, with or without vaccination. *P<0.05, **P<0.01 by 2-way ANOVA with Dunnett's post hoc test, compared to saline. Bars represent mean+SD. In each graph, for each condition (TMP, saline, TMP+vaccine or saline+vaccine) there is a set of two bars with the first bar in each set representing data from female mice and the second bar in each set representing data from male mice.

FIGS. 12A-12F are bar graphs of certain immune cell-counts on Day 38 of female and male mice treated with TMP or saline, with or without vaccination. **P<0.01 by 2-way ANOVA with Dunnett's post hoc test, compared to saline. Bars represent mean+SD. In each graph, for each condition (TMP, saline, TMP+vaccine or saline+vaccine) there is a set of two bars with the first bar in each set representing data from female mice and the second bar in each set representing data from male mice.

FIGS. 13A-13D are bar graphs of cell cycle phases of CD4+T and CD8+ T cell phases on Day 38 in female (FIGS. 13A and 13C) or male mice (FIGS. 13B and 13D) treated with TMP or saline, with or without vaccination. *P<0.05, **P<0.01, ***P<0.001, ****P<0.0001 by 2-way ANOVA with Dunnett's post hoc test, compared to saline. Bars represent mean+SD. In each graph, for each condition (TMP, saline, TMP+vaccine or saline+vaccine) there is a set of four bars representing phases of the cell cycle, in the following order: G0, G1, S and M.

FIGS. 14A-14D are bar graphs of ELISPOT analysis. FIGS. 14A-14B are bar graphs of spleen weights and splenocyte counts, respectively, from WT immunocompetent or immunosuppressed mice not vaccinated or vaccinated with exemplary vaccination. FIGS. 14C-14D are bar graphs of ELISPOT counts of the number of IFNγ/IL5 double-stained colonies.

FIGS. 15A-15L are graphs showing tissue weights of female and male mice treated with TMP or saline, with or without vaccination. FIGS. 15A, 15C, 15E, 15G, 15I, and 15K correspond to data from female mice. FIGS. 15B, 15D, 15F, 15H and 15L correspond to data from male mice. **P<0.01 by 2-way ANOVA with Dunnett's post hoc test, compared to saline.

FIGS. 16A-16D are bar graphs of cell cycle phases of CD4+T and CD8+ T cell phases on Day 17 in female (FIGS. 16A and 16C) or male (FIGS. 16B and 16D) mice treated with TMP or saline, with or without vaccination. *P<0.05, **P<0.01, ***P<0.001, ****P<0.0001 by 2-way ANOVA with Dunnett's post hoc test, compared to saline. Bars represent mean+SD.

FIG. 17 is a bar graph showing serum neutralization of SARS-CoV-2 from WT immunocompetent or immunosuppressed mice which were vaccinated with exemplary vaccination.

FIG. 18 is a bar graph showing anti-RBD serum IgG titer in WT (immunocompetent) or immunosuppressed (IS) BALB/c mice vaccinated with lipid nanoparticles (LNPs) comprising RNAs, wherein the RNAs encoded RBD, an exemplary fusion polypeptide comprising an RBD joined (e.g., directly or indirectly) to Sbi(III-IV), or Spike protein. The IS mice were treated with TMP (as described in Example 6). The RNAs encoding RBD and the fusion polypeptide included N-acetylcytidine (Ac4C) modified nucleosides and the RNAs encoding Spike protein included N1-methylpseudouridine modified nucleosides.

FIG. 19 is a bar graph showing pseudovirus microneutralization titers (pVNT) from WT (immunocompetent) or immunosuppressed (IS) mice vaccinated with LNPs comprising RNAs, wherein the RNAs encoded RBD, an exemplary fusion polypeptide comprising an RBD joined (e.g., directly or indirectly) to Sbi(III-IV), or Spike protein. The RNAs encoding RBD and the fusion polypeptide included N-acetylcytidine (Ac4C) modified nucleosides and the RNAs encoding Spike protein included N1-methylpseudouridine modified nucleosides.

FIG. 20 is a bar graph showing results of an anti-antigen IgG ELISA serology assay performed on serial dilutions of sera from WT (immunocompetent) BALB/c mice vaccinated with lipid nanoparticles (LNPs) comprising RNAs encoding fusion polypeptides comprising an Sbi(III-IV) joined (e.g., directly or indirectly) to an exemplary influenza antigen. The RNA constructs further encoded exemplary signal peptides (e.g., exemplary signal peptide 1 (“SP1”), exemplary signal peptide 2 (“SP2”), or exemplary signal peptide 2 joined (e.g., directly or indirectly) to an exemplary secretion sequence (“SP²_sec”)). Exemplary RNAs encoding the fusion polypeptides included N-acetylcytidine (Ac4C) and 5-hydroxymethyluridine (5hmU) nucleosides. Sera was collected on day 34 (D34). Values were calculated as geometric means, with the error bars corresponding to geometric standard deviation. The graph shows the geometric mean end point serum IgG titer against the exemplary antigen for each vaccine treatment group (titer threshold is set based on untreated control mouse signal at each dilution). Each group had 5 mice.

### CERTAIN DEFINITIONS

About or approximately As used herein, the terms “about” and “approximately,” when used herein in reference to a value, refers to a value that is similar, in context to the referenced value. In general, those skilled in the art, familiar with the context, will appreciate the relevant degree of variance encompassed by “about” or “approximately” in that context. For example, in some embodiments, the term “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administering: As used herein, the term “administering” or “administration” typically refers to administration of a composition to a subject to achieve delivery of an agent that is, or is included in, the composition. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, interdermal, transdermal, etc.), enteral, intra-arterial, intradermal, intragastric, intramedullary, intramuscular, intranasal, intraperitoneal, intrathecal, intravenous, intraventricular, within a specific organ (e.g., intrahepatic), mucosal, nasal, oral, rectal, subcutaneous, sublingual, topical, tracheal (e.g., by intratracheal instillation), vaginal, vitreal, etc. In some embodiments, administration may involve only a single dose. In some embodiments, administration may involve application of a fixed number of doses. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Adjuvant: The term “adjuvant,” as used herein, refers to an agent that modulates and/or enhances an immune response to an agent that elicits an immune response. In some embodiments, an adjuvant is also referred to herein as an immunomodulator. In some embodiments, an adjuvant is administered before, concurrently with or after administration of an agent that elicits an immune response. In some embodiments, an adjuvant and an agent that elicits an immune response are in one composition. In some embodiments, an adjuvant and an agent that elicits an immune response are in different compositions. In some embodiments, an adjuvant is or comprises a nucleic acid, polypeptide, polysaccharide, or small molecule. In some embodiments, an adjuvant is or comprises a complement binding domain. In some embodiments, an adjuvant is or comprises a C3d binding domain. In some embodiments, an adjuvant is or comprises a domain III of Sbi immunoglobulin-binding protein of Staphylococcus aureus, or a functional fragment or variant thereof. In some embodiments, an adjuvant is or comprises a domain IV of Sbi immunoglobulin-binding protein of Staphylococcus aureus, or a functional fragment or variant thereof. In some embodiments, an adjuvant comprises both a domain III and a domain IV of the Sbi of Staphylococcus aureus, or a functional fragment or a variant thereof.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen comprises at least one epitope of a target protein. In some embodiments, an epitope may be a linear epitope. In some embodiments, an epitope may be a conformational epitope. In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer]) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen. In some embodiments, an antigen comprises a wild-type or variant sequence (e.g., an antigen variant). In some embodiments, an antigen comprises a full-length version of the antigen or a portion of the antigen (e.g., an antigen fragment or an antigen fragment variant).

Antigen fragment: An “antigen fragment” is used herein to refer to a fragment of an antigen that is not the full-length antigen but comprises at least one epitope of the antigen. In some embodiments, an epitope is or comprises an epitope presented by MHC Class I. In some embodiments, an epitope is or comprises an epitope presented by MHC Class II. In some embodiments, an antigen fragment is a polypeptide. In some embodiments, an antigen fragment is encoded by a polynucleotide encoding an antigen fragment. In some embodiments, a polypeptide antigen fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 200, 250, 300 or more monomeric units (e.g., residues) as found in a target protein antigen polypeptide. In some embodiments, a polypeptide antigen fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in a target protein antigen polypeptide. In some embodiments, a polypeptide antigen fragment comprises or consists of no more than about 50%, 40%, 30%, 20%, 10%, or 5% of the monomeric units (e.g., residues) found in a target protein antigen polypeptide.

Antigen fragment variant: As used herein, the term “antigen fragment variant” refers to an antigen fragment that shows significant sequence and/or structural identity with an antigen fragment but differs in sequence and/or structure from the antigen fragment in the presence or level of one or more chemical moieties as compared to the antigen fragment. In some embodiments, an antigen fragment variant differs functionally from an antigen fragment. In some embodiments, an antigen fragment variant does not differ functionally from an antigen fragment. In some embodiments, an antigen fragment variant differs from an antigen fragment as a result of one or more differences in amino acid sequence and/or one or more differences in chemical moieties (e.g., carbohydrates, lipids, etc.) covalently attached to the polypeptide backbone.

Antigen variant: As used herein, the term “antigen variant” refers to an antigen that shows significant structural identity with a target protein antigen but differs structurally from the target protein antigen in the presence or level of one or more chemical moieties as compared to the target protein antigen. In some embodiments, an antigen variant differs functionally from a target protein antigen. In some embodiments, an antigen variant does not differ functionally from a target protein antigen. In some embodiments, an antigen variant comprises an epitope of a target protein antigen. In some embodiments, an antigen variant differs from a target protein antigen as a result of one or more differences in amino acid sequence and/or one or more differences in chemical moieties (e.g., carbohydrates, lipids, etc.) covalently attached to the polypeptide backbone.

Comparable: As used herein, the term “comparable” refers to two or more agents (e.g., entities or set(s) of conditions), situations, etc., that may not be identical to one another but that are sufficiently similar to permit comparison therebetween so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Delivery/contacting: As used interchangeably herein, the term “delivery,” “delivering,” or “contacting” refers to introduction of a polynucleotide (e.g., as described herein) or a fusion polypeptide (e.g., as described herein) into a target cell. A target cell can be cultured in vitro or ex vivo or be present in a subject (in vivo). Methods of introducing a polynucleotide (e.g., as described herein) or a fusion polypeptide (e.g., as described herein) into a target cell can vary with in vitro, ex vivo, or in vivo applications. In some embodiments, a polynucleotide (e.g., as described herein) or a fusion polypeptide (e.g., as described herein) can be introduced into a target cell in a cell culture by in vitro transfection. In some embodiments, a polynucleotide (e.g., as described herein) or a fusion polypeptide (e.g., as described herein) can be introduced into a target cell via delivery vehicles (e.g., nanoparticles, liposomes, and/or complexation with a cell-penetrating agent). In some embodiments, a polynucleotide (e.g., as described herein) or a fusion polypeptide (e.g., as described herein) can be introduced into a target cell in a subject by administering a polynucleotide (e.g., as described herein) or a fusion polypeptide (e.g., as described herein) to a subject.

Encode: As used herein, the team “encode” or “encoding” refers to a first molecule that is produced by a second molecule, wherein the sequence information of the second molecule determines the sequence of the first molecule. For example, a first molecule can have a defined sequence of nucleotides (e.g., a polyribonucleotide) or a defined sequence of amino acids which are determined by the sequence of the second molecule (e.g., a polynucleotide). For example, a DNA molecule (e.g., a second molecule) can encode an RNA molecule (e.g., a first molecule, for example by a transcription process that includes a DNA-dependent RNA polymerase enzyme) or a polypeptide (e.g., a first molecule for example by a transcription and a translation process). An RNA molecule (e.g., a second molecule) can encode a polypeptide (e.g., a first molecule for example by a translation process). Thus, a gene, a cDNA, or an RNA molecule encodes a polypeptide if transcription and translation of RNA corresponding to that gene produces the polypeptide in a cell or other biological system.

Functional: As used herein, the term “functional” is used to refer to a form or fragment of an entity that exhibits a particular property and/or activity.

Fragment: A “fragment” of a material or entity as described herein has a structure that includes a discrete portion of the whole, but lacks one or more moieties found in the whole. In some embodiments, a fragment consists of such a discrete portion. In some embodiments, a fragment consists of or comprises a characteristic structural element or moiety found in the whole. In some embodiments, a fragment comprises a polynucleotide fragment. In some embodiments, a fragment comprises a polypeptide fragment. In some embodiments, a polynucleotide fragment or a polypeptide fragment comprises or consists of at least 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 275, 300, 325, 350, 375, 400, 425, 450, 475, 500 or more monomeric units (e.g., residues) as found in the whole polynucleotide or whole polypeptide. In some embodiments, a polynucleotide fragment or a polypeptide fragment comprises or consists of at least about 5%, 10%, 15%, 20%, 25%, 30%, 25%, 40%, 45%, 50%, 55%, 60%, 65%, 70%, 75%, 80%, 85%, 90%, 95%, 96%, 97%, 98%, 99% or more of the monomeric units (e.g., residues) found in the whole polynucleotide or whole polypeptide. The whole polypeptide or whole polynucleotide may in some embodiments be referred to as the “parent” of the polynucleotide fragment or polypeptide fragment.

Immunosuppressed subject: As used herein, the term “immunosuppressed subject” refers to a subject having an immune response that is reduced, e.g., suppressed or compromised, compared to a reference. In some embodiments, an immune response that is reduced, e.g., suppressed or compromised, is reduced by about 5% to about 100% compared to a reference. In some embodiments, a reference comprises an immune response of a subject prior to initiation of a therapy. In some embodiments, a reference comprises an immune response of a healthy subject. In some embodiments, a reference comprises an immune response of a population of comparable healthy subjects. In some embodiments, an immunosuppressed subject is an immunocompromised subject. As described herein, in some instances, an immunosuppressed subject is receiving an agent (e.g., drug) to suppress an immune response in the subject. In some situations, an immunosuppressed subject has a disease, disorder, or condition that suppresses the subject's immune system, e.g., one or more genetic mutations that suppresses a subject's immune system. Methods for determining whether a subject is immunosuppressed or diagnosing a subject as being immunosuppressed are known in the art. For example, in some embodiments, a subject may have a reduced number or percentage of certain immune cells (e.g., B cells, T cells, macrophages, NK cells, etc.). In some embodiments, a subject may have certain immune cells (e.g., B cells, T cells, macrophages, NK cells, etc.) that have reduced functionality. In some embodiments, immunosuppression is characterized by a frequency or intensity of diseases, disorders, symptoms or conditions experienced by a subject in a set time frame. In some embodiments, immunosuppression is characterized by a reduced immune response (e.g., in magnitude and/or timing) to one or more pathogens by a subject as compared to a reference, e.g., a healthy subject.

Nucleic acid/Oligonucleotide/Polynucleotide: As used herein, the terms “nucleic acid” and “polynucleotide” and “oligonucleotide” are used interchangeably, and refer to a polymer of 3 nucleotides or more. In some embodiments, a nucleic acid comprises DNA. In some embodiments, a nucleic acid comprises RNA. In some embodiments, a nucleic acid comprises messenger RNA (mRNA). In some embodiments, a nucleic acid is single stranded. In some embodiments, a nucleic acid is double stranded. In some embodiments, a nucleic acid comprises both single and double stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural nucleosides (e.g., adenosine, cytidine, guanosine, uridine, thymidine, deoxyadenosine, deoxycytidine, deoxyguanosine, deoxythymidine, deoxyuridine). In some embodiments, a nucleic acid comprises one or more, or all, non-natural nucleotides. In some embodiments, a non-natural nucleoside comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, a nucleoside comprising a methylated base, a nucleoside comprising an intercalated base, and combinations thereof). In some embodiments, a non-natural nucleoside comprises a modified nucleoside, e.g., as described herein. In some embodiments, a non-natural nucleoside comprises N4-acetylcytidine, 5-hydroxymethyluridine and/or N1-methylpseudouridine. In some embodiments, a non-natural nucleoside comprises one or more modified sugars (e.g., 2′-fluororibose, ribose, 2′-deoxyribose, arabinose, and hexose) as compared to those in natural nucleosides. In some embodiments, a non-natural nucleoside comprises a 2′-O-acetylated ribose. As used herein, “adenosine nucleosides” encompasses a natural adenosine nucleoside, as well as modified adenosine nucleosides. Accordingly, when a percentage of adenosine nucleosides is provided herein, the percentage is based on a total number of natural and modified adenosine nucleosides. Similarly, “cytidine nucleosides” encompasses a natural cytidine nucleoside, as well as modified cytidine nucleosides (e.g., N4-acetylcytidine or 2′-O-acetylated N4-acetylcytidine); “guanosine nucleosides” encompasses a natural guanosine nucleoside, as well as modified guanosine nucleosides; and “uridine nucleosides” encompasses a natural uridine nucleoside, as well as modified uridine nucleosides (e.g., 5-hydroxymethyluridine or 2′-O-acetylated 5-hydroxymethyluridine). Further, as used herein, “adenine nucleobase” encompasses a natural adenine nucleobase, as well as modified adenine nucleobase; “cytosine nucleobase” encompasses a natural cytosine nucleobase, as well as modified cytosine nucleobase (e.g., N4-acetylcytosine); “guanine nucleobase” encompasses a natural guanine nucleobase, as well as modified guanine nucleobase; and “uracil nucleobase” encompasses a natural uracil nucleobase, as well as modified uracil nucleobase (e.g., 5-hydroxymethyluracil). In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 nucleotides long. When a number of nucleotides is used as an indication of size, e.g., of a polynucleotide, a certain number of nucleotides refers to the number of nucleotides on a single strand, e.g., of a polynucleotide.

Polypeptide: The term “polypeptide”, as used herein, generally has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof.

RNA oligonucleotide: As used herein, the term “RNA oligonucleotide” refers to an oligonucleotide of ribonucleotides. In some embodiments, an RNA oligonucleotide is single stranded. In some embodiments, an RNA oligonucleotide is double stranded. In some embodiments, an RNA oligonucleotide comprises both single and double stranded portions. In some embodiments, an RNA oligonucleotide can comprise a backbone structure as described in the definition of “Nucleic acid/Oligonucleotide” above. An RNA oligonucleotide can be a regulatory RNA (e.g., siRNA, microRNA, etc.), or a messenger RNA (mRNA) oligonucleotide. In some embodiments an RNA oligonucleotide can comprise at its 3′ end a poly(A) region. In some embodiments an RNA oligonucleotide comprises at its 5′ end a cap structure, e.g., for recognizing and attachment of an RNA to a ribosome to initiate translation. In some embodiments, a polynucleotide comprises an RNA oligonucleotide. When a number of ribonucleotides is used as an indication of size, e.g., of a polynucleotide, a certain number of nucleotides refers to the number of ribonucleotides on a single strand.

Subject: As used herein, the term “subject” refers an organism, typically a mammal (e.g., a human). In some embodiments, a subject is an immunosuppressed subject. In some embodiments, a subject is suffering from a disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Variant: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide.

Standard techniques may be used for recombinant DNA, oligonucleotide synthesis, e.g., RNA synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Green and Sambrook, Molecular Cloning: A Laboratory Manual (4^{th} ed., Cold Spring Harbor Laboratory Press, Cold Spring Harbor, N.Y. (2012)), which is incorporated herein by reference for any purpose.

### DETAILED DESCRIPTION OF CERTAIN EMBODIMENTS

Eliciting an immune response, such as a productive immune response to a vaccine, can be challenging in immunosuppressed subjects (e.g., immunocompromised subjects). Immunosuppressed subjects include transplant recipients and patients treated with immunosuppressants such as chemotherapy or radiation therapy. The immune system of immunosuppressed subjects is often kept suppressed to manage a disease or disorder such as cancer or transplant rejection. Immunosuppressed subjects also include subjects that have a weakened immune system due to a disease, disorder or condition. As a consequence, a suppressed immune response in such subjects is often unable to generate a productive and meaningful immune response to vaccination.

This disclosure provides technologies, such as fusion polypeptides and polynucleotides encoding fusion polypeptides. In some embodiments, a fusion polypeptide comprises antigens joined (e.g., directly or indirectly) joined to a complement C3d-binding region. In some embodiments, a complement C3d-binding region of a fusion polypeptide acts as an adjuvant, e.g., an immunomodulator. In some embodiments, the provided technologies can address challenges associated with diminished immune responses in immunosuppressed subjects. The present disclosure provides the recognition that vaccinating an immunosuppressed subject with a polynucleotide encoding an antigen joined (e.g., directly or indirectly) to a complement C3d-binding region (e.g., in a fusion polypeptide provided herein) can enhance the immunogenicity of the antigen and provide an increased immune response (e.g., titers of the resulting antibody response) while minimizing undesired immunogenicity from the delivered polynucleotide itself. For example, delivery of fusion polynucleotides encoding fusion polypeptides as disclosed herein can directly stimulate and/or drive uptake of the encoded fusion polypeptide by B cells, while minimizing non-specific inflammation at the site of polynucleotide administration and/or expression. As an example, this disclosure provides compositions and uses of fusion polypeptides (and fusion polynucleotide encoding the same) comprising an exemplary antigen joined (e.g., directly or indirectly) to an Sbi fragment (Sbi III-IV) which have enhanced antigenicity and/or stimulate a productive humoral immune response in immunosuppressed subjects (see Examples 6 and 7).

In some embodiments, an immunosuppressed subject generates a T cell response that is less functional compared to a non-immunosuppressed subject, e.g., a healthy subject. In some embodiments, an immunosuppressed subject does not generate a functional T cell response.

In some embodiments, a humoral immune response stimulated by a composition disclosed herein occurs in the absence of T cells (e.g., absence of functional T cells) or in the presence of T cells that are defective in stimulating an immune response. In some embodiments, a composition disclosed herein stimulates B cells in a subject, e.g., in the absence of T cells, to elicit a productive humoral immune response.

In some embodiments, a complement C3d-binding region of a Sbi protein from Staphylococcus aureus is an adjuvant. In some embodiments, an antigen joined (e.g., directly or indirectly) to a complement C3d-binding region serves as a synthetic immunological synapse, mimicking natural viral infection to drive a strong and appropriate immune response. In some embodiments, an antigen joined (e.g., directly or indirectly) to a complement C3d-binding region allows small antigens that lack MHC-presented peptides to elicit meaningful humoral response.

### Immunosuppressed Subjects and Related Disorders

Among other things, disclosed herein are methods of enhancing immunogenicity against an antigen and/or methods of inducing an immune response against an antigen in a subject, e.g., an immunosuppressed subject. Immunosuppressed subjects as described herein can have, or be suspected of having, or be predisposed to one or more disorders, e.g., as described herein, and/or have received or will be receiving one or more treatments, e.g., as described herein. For example, an immunosuppressed subject can be a subject who: (1) has received, is receiving, or will be receiving a transplant, and (2) has one or more disorders disclosed herein. As another example, an immunosuppressed subject can be a subject who: (1) has a suppressed or compromised immune system due to a drug, e.g., an immunosuppressive therapy (e.g., an organ transplant conditioning regimen, a therapy that suppresses an immune system (e.g., an antibody therapy), a B-cell targeting therapy, a T-cell targeting therapy, a chemotherapy, a radiation therapy, a cancer therapy, a treatment for an inflammatory disease and/or a treatment for an autoimmune disease), and (2) has one or more disorders disclosed herein.

In some embodiments, an immunosuppressed subject has received, is receiving, or will be receiving one or more transplants. In some embodiments, an immunosuppressed subject who has received, is receiving, or will be receiving one or more transplants can also be administered one or more therapeutic agents and/or be subjected to one or more procedures, e.g., dialysis.

In some embodiments, an immunosuppressed subject has received, is receiving, or will be receiving one or more dialysis treatments. In some embodiments, a dialysis treatment is or comprises hemodialysis. In some embodiments, a dialysis treatment is or comprises peritoneal dialysis.

In some embodiments, one or more transplants described herein is an organ transplant. In some embodiments, an organ transplant comprises: a kidney transplant, a liver transplant, a heart transplant, a lung transplant, a pancreas transplant, a stomach transplant, an intestine transplant, or any combination thereof.

In some embodiments, an immunosuppressed subject has received, is receiving, or will be receiving a kidney transplant.

In some embodiments, an immunosuppressed subject has received, is receiving, or will be receiving a liver transplant.

In some embodiments, an immunosuppressed subject has received, is receiving, or will be receiving a heart transplant.

In some embodiments, an immunosuppressed subject has received, is receiving, or will be receiving lung transplant.

In some embodiments, an immunosuppressed subject has received, is receiving, or will be receiving a pancreas transplant.

In some embodiments, an immunosuppressed subject has received, is receiving, or will be receiving a stomach transplant.

In some embodiments, an immunosuppressed subject has received, is receiving, or will be receiving an intestine transplant. In some embodiments, an immunosuppressed subject who has received, is receiving, or will be receiving one or more transplants can also be administered one or more therapeutic agents and/or be subjected to one or more procedures, e.g., dialysis. In some embodiments, an immunosuppressed subject has received, is receiving, or will be receiving one or more dialysis treatments. In some embodiments, a dialysis treatment is or comprises hemodialysis. In some embodiments, a dialysis treatment is or comprises peritoneal dialysis.

In some embodiments, one or more transplants described herein is a cell transplant. In some embodiments, a cell transplant is a transplant of a population of stem cells (e.g., hematopoietic stem cells, induced pluripotent stem cells, or embryonic stem cells), immune cells, or any combination thereof. In some embodiments, a cell transplant is a transplant of a population of bone marrow cells, blood cells, or any combination thereof.

In some embodiments, a cell transplant is a transplant of a population of engineered cells.

In some embodiments, a cell transplant is a transplant of a population of non-engineered cells.

In some embodiments, an immunosuppressed subject has received, is receiving, or will be receiving a transplant of a population of stem cells (e.g., hematopoietic stem cells, induced pluripotent stem cells, or embryonic stem cells), immune cells, or any combination thereof.

In some embodiments, an immunosuppressed subject has received, is receiving, or will be receiving a transplant of a population of bone marrow cells, blood cells, or any combination thereof.

In some embodiments, an immunosuppressed subject has received, is receiving, or will be receiving a transplant of a population of engineered cells.

In some embodiments, an immunosuppressed subject has received, is receiving, or will be receiving a transplant of a population of non-engineered cells.

In some embodiments, one or more transplants described herein is a tissue transplant. In some embodiments, a tissue transplant comprises skin tissue transplant, bone tissue transplant, cartilage tissue transplant, adrenal tissue transplant, corneal tissue transplant, or any combination thereof.

In some embodiments, a transplant is an allogeneic transplant.

In some embodiments, an immunosuppressed subject has received, is receiving, or will be receiving a skin tissue transplant.

In some embodiments, an immunosuppressed subject has received, is receiving, or will be receiving a bone tissue transplant.

In some embodiments, an immunosuppressed subject has received, is receiving, or will be receiving a cartilage tissue transplant.

In some embodiments, an immunosuppressed subject has received, is receiving, or will be receiving an adrenal tissue transplant.

In some embodiments, an immunosuppressed subject has received, is receiving, or will be receiving a corneal tissue transplant.

In some embodiments, an immunosuppressed subject has or has been diagnosed with one or more diseases, e.g., as described herein. In some embodiments, an immunosuppressed subject has received, is receiving or will receive one or more treatments for one or more diseases, e.g., as described herein.

In some embodiments, an immunosuppressed subject has or has been diagnosed with a rare disease, a lung disease, a liver disease, a kidney disease, a blood disorder, an autoimmune disease, an immunodeficiency disorder, a cardiovascular disorder, a cancer, or any combination thereof.

In some embodiments, an immunosuppressed subject has or has been diagnosed with a rare disease, e.g., cystic fibrosis. In some embodiments, an immunosuppressed subject has received, is receiving or will receive one or more treatments for a rare disease, e.g., cystic fibrosis. In some embodiments, treatments for cystic fibrosis include cystic fibrosis transmembrane conductance regulator (“CFTR”) modulator therapy (e.g., ivacaftor, lumacaftor-ivacaftor, tezacaftor-ivacaftor, ETI, etc.). See, for example, Southern, K. et al., “Standards for the care of people with cystic fibrosis; establishing and maintaining health,” Journal of Cystic Fibrosis, Volume 23, Issue 1, 12-28, the entire contents of which are hereby incorporated by reference in its entirety.

In some embodiments, an immunosuppressed subject has or has been diagnosed with a lung disease, e.g., chronic lung disease, idiopathic pulmonary fibrosis (“IPF”), pulmonary arterial hypertension (“PAH”), chronic obstructive pulmonary disease (“COPD”), or emphysema. In some embodiments, an immunosuppressed subject has received, is receiving or will receive one or more treatments for a lung disease. In some embodiments, treatments for lung disease include bronchodilators (e.g., short-acting beta-agonists, long-acting beta-agonists), inhaled corticosteroids, and/or combinations thereof. See, for example, Nici L, et al. Pharmacologic management of chronic obstructive pulmonary disease: An official American Thoracic Society clinical practice guideline. American Journal of Respiratory and Critical Care Medicine. 2020; doi:10.1164/rccm.202003-0625ST, the entire contents of which are hereby incorporated by reference in its entirety.

In some embodiments, an immunosuppressed subject has or has been diagnosed with autoimmune disease, e.g., diabetes, systemic lupus erythematosus (“SLE”) or multiple sclerosis. In some embodiments, an immunosuppressed subject has received, is receiving or will receive one or more treatments for an autoimmune disease. In some embodiments, a treatment for diabetes includes insulin therapy. See for example, American Diabetes Association Professional Practice Committee; 9. Pharmacologic Approaches to Glycemic Treatment: Standards of Care in Diabetes-2024. Diabetes Care 1 Jan. 2024; 47 (Supplement_1): S158-S178, the entire contents of which are hereby incorporated by reference in its entirety. In some embodiments, exemplary treatments for SLE includes hydroxychloroquine, corticosteroids (e.g., prednisone), immunosuppressants (e.g., azathioprine, cyclophosphamide, mycophenolate, methotrexate), and/or combinations thereof. See, for example, Fanouriakis A, et al, “EULAR recommendations for the management of systemic lupus erythematosus: 2023 update,” Annals of the Rheumatic Diseases 2024; 83:15-29, the entire contents of which are hereby incorporated by reference in its entirety. In some embodiments, exemplary treatments for multiple sclerosis include steroids, immunosuppressants (e.g., mitoxantrone, cyclophosphamide, methotrexate). See, for example, Hauser S L, Cree B A C. Treatment of Multiple Sclerosis: A Review. Am J Med. 2020 December; 133(12):1380-1390.e2. doi: 10.1016/j.amjmed.2020.05.049. Epub 2020 Jul. 17. PMID: 32682869, the entire contents of which are incorporated by reference in its entirety.

In some embodiments, an immunosuppressed subject has or has been diagnosed with liver disease, e.g., chronic liver disease (e.g., cirrhosis). In some embodiments, an immunosuppressed subject has received, is receiving or will receive one or more treatments for a liver disease. In some embodiments, treatment for chronic liver disease includes anti-viral agents.

In some embodiments, an immunosuppressed subject has or has been diagnosed with kidney disease, e.g., end-stage renal disease, chronic kidney disease, or IgA nephropathy. In some embodiments, an immunosuppressed subject has received, is receiving or will receive one or more treatments for kidney disease. In some embodiments, exemplary treatments for kidney diseases comprise angiotensin-converting-enzyme (“ACE”) inhibitors, angiotension receptor blockers (“ARBs”), empagliflozin, dialysis, and/or combinations thereof. See, for example, Breyer M D, Susztak K. Developing Treatments for Chronic Kidney Disease in the 21st Century. Semin Nephrol. 2016 November; 36(6):436-447. doi: 10.1016, the entire contents of which are hereby incorporated by reference in its entirety.

In some embodiments, an immunosuppressed subject has or has been diagnosed with a cardiovascular disease. In some embodiments, an immunosuppressed subject has received, is receiving or will receive one or more treatments for a cardiovascular disease. In some embodiments, exemplary treatments for cardiovascular include anticoagulants, antiplatelet agents and dual antiplatelet therapy, ACE inhibitors, angiotensin II receptor blockers, angiotensin receptor-neprilysin inhibitors, beta blockers, calcium channel blockers, cholesterol-lowering medications, digitalis preparations, diuretics, vasodilators, or combinations thereof.

In some embodiments, an immunosuppressed subject has or has been diagnosed with a blood disorder. In some embodiments, an immunosuppressed subject has received, is receiving or will receive one or more treatments for a blood disorder. In some embodiments, exemplary treatments for a blood disorder includes blood transfusions, platelet transfusions, anticoagulants, growth factor supplements, corticosteroids,

In some embodiments, an immunosuppressed subject has or has been diagnosed with a neurological disease, e.g., neuromyelitis optica (“NMO”), Guillain-Barre syndrome (“GBS”). In some embodiments, an immunosuppressed subject has received, is receiving or will receive one or more treatments for a neurological disease.

In some embodiments, an immunosuppressed subject has or has been diagnosed with an immunodeficiency disorder, e.g., human immunodeficiency virus (“HIV”) infection, acquired immune deficiency syndrome (“AIDS”), and other various primary immunodeficiency disorders (“PIDDs”). In some embodiments, an immunosuppressed subject has received, is receiving or will receive one or more treatments for an immunodeficiency disorder. In some embodiments, exemplary treatments for HIV include nucleoside reverse transcriptase inhibitors (“NRTIs”), non-nucleoside reverse transcriptase inhibitors (“NNRTIs”), protease inhibitors (“PIs”), fusion inhibitors, CCR5 antagonists, integrase strand transfer inhibitors (“INSTIs”), attachment inhibitors, post-attachment inhibitors, capsid inhibitors, combinations thereof. See, for example, Phanuphak N, Gulick R M. HIV treatment and prevention 2019: current standards of care. Curr Opin HIV AIDS. 2020 January; 15, the entire contents of which are hereby incorporated by reference in its entirety.

In some embodiments, an immunosuppressed subject has or has been diagnosed with a cancer, e.g., a blood cancer or a solid cancer. In some embodiments, a blood cancer is a leukemia, a lymphoma (e.g., Hodgkin's lymphoma, non-Hodgkin's lymphoma), or a myeloma (e.g., multiple myeloma). In some embodiments, an immunosuppressed subject has received, is receiving or will receive one or more treatments for cancer, e.g., chemotherapy and/or radiation therapy.

In some embodiments, an immunosuppressed subject is more than 65 years old. In some embodiments, an immunosuppressed subject is about 65 years to about 100 years old.

In some embodiments, an immunosuppressed subject has received or is receiving an immunosuppressive therapy. In some embodiments, an immunosuppressive therapy comprises: an organ transplant conditioning regimen, a therapy that suppresses an immune system, a B-cell targeting therapy, a T-cell targeting therapy, a chemotherapy, a radiation therapy, a cancer therapy, a treatment for an inflammatory disease and/or a treatment for an autoimmune disease.

In some embodiments, an immunosuppressed subject has received or is receiving a B cell targeting therapy (e.g., rituximab, or belimumab).

In some embodiments, an organ transplant conditioning regimen comprises a calcineurin inhibitor, an antiproliferative agent, a steroid, an mTOR inhibitor or any combination thereof. In some embodiments, a calcineurin inhibitor comprises tacrolimus. In some embodiments, an antiproliferative agent comprises mycophenolate mofetil. In some embodiments, a steroid comprises prednisone. In some embodiments, an organ transplant conditioning regimen comprises tacrolimus, mycophenolate mofetil and prednisone, or any combination thereof.

### Exemplary Antigens

The present disclosure provides antigens (including, e.g., antigen fragments, antigen variants, and/or antigen fragment variants). In some embodiments, antigens (e.g., antigen variants, antigen fragments or antigen fragment variants) provided herein comprise an epitope. The present disclosure further provides compositions comprising antigens (e.g., antigen variants, antigen fragments or antigen fragment variants described herein). In some embodiments, an antigen (e.g., an antigen variant, an antigen fragment or an antigen fragment variant) is joined (e.g., directly or indirectly) a C3d binding polypeptide. In some embodiments, a fusion polypeptide provided herein comprises an antigen (e.g., an antigen variant, an antigen fragment or an antigen fragment variant) joined (e.g., directly or indirectly) to a C3d binding polypeptide.

In some embodiments, fusion polypeptides disclosed herein (e.g., encoded by fusion polynucleotides disclosed herein) comprise one or more antigens (e.g., antigen fragments, antigen variants, and/or antigen fragment variants). In some embodiments, one or more antigens are from one pathogen. In some embodiments, one or more antigens are from different pathogens (e.g., 2 or more different pathogens). In some embodiments, fusion polypeptides disclosed herein comprise 1, 2, 3, or 4 antigens from one pathogen or from one or more different pathogens.

In some embodiments, a fusion polypeptide disclosed herein comprises at least 2, 3, 4, 5, or 10 of the same antigen from one pathogen. In some embodiments, a fusion polypeptide disclosed herein comprises at least 2, 3, 4, 5, or 10 different antigens from one pathogen.

In some embodiments, a fusion polypeptide disclosed herein comprises at least 2, 3, 4, 5, or 10 of antigens from different pathogens (e.g., 2 or more different pathogens). In some embodiments, at least 2, 3, 4, 5, or 10 antigens are each a different antigen, e.g., from a different target protein.

In some embodiment, a fusion polypeptide comprises more than one antigen (e.g., more than one antigen variant, antigen fragment or antigen fragment variant) joined (e.g., directly or indirectly) to a C3d binding polypeptide In some embodiments, a fusion polypeptide comprises 2, 3, 4, 5, or 10 antigens (e.g., antigen variants, antigen fragments or antigen fragment variants) joined (e.g., directly or indirectly) to a C3d binding polypeptide.

In some embodiments, an antigen (e.g., an antigen variant, an antigen fragment or an antigen fragment variant) comprises an epitope (e.g., T cell epitope) of a target protein antigen. In some embodiments, an antigen (e.g., an antigen variant, an antigen fragment or an antigen fragment variant) comprises a portion of an epitope (e.g., T cell epitope) of a target protein antigen.

In some embodiments, an antigen (e.g., an antigen variant, an antigen fragment or an antigen fragment variant) does not comprise one or more epitopes (e.g., T cell epitope) of a wild-type, full-length version of the antigen.

In some embodiments, an antigen fragment has an amino acid sequence length of at least 5%, at least 10%, at least 20%, at least 30%, at least 40%, at least 50%, at least 60%, at least 70%, at least 80%, at least 90% or at least 95% compared to the amino acid sequence length of a wild-type, full length target protein antigen. In some embodiments, an antigen fragment has an amino acid sequence length of no more than 50% of the amino acid sequence length of a wild-type, full length target protein antigen. In some embodiments, an antigen fragment has an amino acid sequence length of no more than 40%, no more than 30%, no more than 20%, no more than 10% or no more than 5% of the amino acid sequence length of a wild-type, full length target protein antigen.

In some embodiments, an antigen fragment has about 10-300 amino acid residues in length. In some embodiments, an antigen fragment has at least 10 amino acid residues in length. In some embodiments, an antigen fragment has less than about 300 amino acid residues in length. In some embodiments, an antigen fragment has 10-300, 10-250, 10-200, 10-150, 10-100, 10-90, 10-80, 10-70, 10-60, 10-50, 10-40, 10-30, 10-20, 20-300, 30-300, 40-300, 50-300, 60-300, 70-300, 80-300, 90-100, 100-300, 150-300, 200-300, 250-300, 20-250, 30-200, 40-150, 50-100, 60-90, or 70-80 amino acids residues in length. In some embodiments, an antigen fragment has about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, or about 300 amino acids residues in length.

In some embodiments, an antigen variant or antigen fragment variant amino acid sequence length is identical to the amino acid sequence length of a wild-type, full length target protein antigen.

In some embodiments, an antigen variant or antigen fragment variant comprises at least one modified amino acid compared to a reference target protein antigen, e.g., a wild-type target protein antigen. In some embodiments, a modified amino acid comprises N-linked glycosylation. In some embodiments, an antigen variant or antigen fragment variant comprises at least one amino acid mutation compared to a reference target protein antigen, e.g., a wild-type target protein antigen. In some embodiments, an antigen variant or antigen fragment variant comprises 2, 3, 4, 5, 6, 7, 8, 9, 10, 15, 20, 30, 40 or 50 amino acid mutations compared to a reference target protein antigen, e.g., a wild-type target protein antigen. In some embodiments, the mutation introduces a Serine, a Threonine, an Alanine, or an amino acid at a particular position which is different from the amino acid present at that position in a reference target protein antigen, e.g., a wild-type target protein antigen. In some embodiments, the mutation prevents formation of a disulfide bond.

In some embodiments, an antigen fragment is characterized in that when expressed in vivo, it binds to a Major Histocompatibility Complex (MHC) molecule.

In some embodiments, an antigen fragment is characterized in that when expressed in vivo, it does not bind to an MHC molecule.

In some embodiments, an MHC molecule is or comprises an MHC class I molecule (e.g., HLA class I) or an MHC class II (e.g., HLA class II) molecule.

In some embodiments, an antigen fragment is characterized in that when expressed in vivo, it folds into a three-dimensional conformation that is substantially identical to the three-dimensional conformation of the antigen fragment as it is in its native position in the target protein antigen.

In some embodiments, an antigen fragment, antigen variant or antigen fragment variant further comprises an amino acid sequence from a second target protein antigen.

### Exemplary Target Protein Antigens

Among other things, provided herein are fusion polypeptides comprising one or more antigens (e.g., one or more antigen variants, one or more antigen fragments or one or more antigen fragment variants) comprising one or more epitopes of one or more target protein antigens. Also provided herein are polynucleotides encoding fusion polypeptides comprising one or more antigens (e.g., one or more antigen variants, one or more antigen fragments or one or more antigen fragment variants) comprising one or more epitopes of one or more target protein antigens.

In some embodiments, a target protein antigen is or comprises an infectious disease antigen. In some embodiments, an infectious disease antigen is or comprises a viral antigen, a bacterial antigen, a fungal antigen, or any combination thereof. In some embodiments, a viral antigen is or comprises an influenza antigen. In some embodiments, a viral antigen is or comprises a coronavirus polypeptide. In some embodiments, a coronavirus polypeptide is or comprises a SARS-CoV-2 protein, e.g., as described herein.

In some embodiments, a target protein antigen is or comprises a cancer antigen.

### SARS-CoV-2 Antigens

Exemplary antigens that can be included in any of the fusion polypeptides, polynucleotides encoding fusion polypeptides, compositions, methods or uses disclosed herein include one or more SARS-CoV-2 polypeptides. In some embodiments, a target protein antigen disclosed herein is or comprises a SARS-CoV-2 antigen. In some embodiments, a SARS-CoV-2 antigen is a Spike glycoprotein (SARS-CoV-2 S) polypeptide or antigenic fragment thereof; an Envelope protein (SARS-CoV-2 E) polypeptide or antigenic fragment thereof; a Membrane protein (SARS-CoV-2 M) polypeptide or antigenic fragment thereof; a nucleocapsid protein (SARS-CoV-2 N) polypeptide or antigenic fragment thereof; an accessory factor polypeptide or antigenic fragment thereof; or any combination thereof.

In some embodiments, an antigen (e.g., an antigen an antigen variant, an antigen fragment, or an antigen fragment variant) comprises an epitope from a target protein antigen which is or comprises a SARS-CoV-2 polypeptide. In some embodiments, an antigen, e.g., an antigen fragment, an antigen variant or an antigen fragment variant, comprises an epitope from a SARS-CoV-2 Spike glycoprotein (SARS-CoV-2 S) polypeptide or a fragment thereof (e.g., RBD).

The SARS-CoV-2 S polypeptide is referenced by Gene ID: 43740568 and/or NCBI RefNC_045512.2. An amino acid sequence for SARS-CoV-2 S polypeptide is provided by SEQ ID NO: 14:

MFVFLVLLPLVSSQCVNLTTRTQLPPAYTNSFTRGVYYPDKVFR
SSVLHSTQDLFLPFFSNVTWFHAIHVSGTNGTKRFDNPVLPFNDGVYFASTEKSNIIR
GWIFGTTLDSKTQSLLIVNNATNVVIKVCEFQFCNDPFLGVYYHKNNKSWMESEFRVY
SSANNCTFEYVSQPFLMDLEGKQGNFKNLREFVFKNIDGYFKIYSKHTPINLVRDLPQ
GFSALEPLVDLPIGINITRFQTLLALHRSYLTPGDSSSGWTAGAAAYYVGYLQPRTFL
LKYNENGTITDAVDCALDPLSETKCTLKSFTVEKGIYQTSNFRVQPTESIVRFPNITN
LCPFGEVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKVGGNYN
YLYRLFRKSNLKPFERDISTEIYQAGSTPCNGVEGFNCYFPLQSYGFQPTNGVGYQPY
RVVVLSFELLHAPATVCGPKKSTNLVKNKCVNFNFNGLTGTGVLTESNKKFLPFQQFG
RDIADTTDAVRDPQTLEILDITPCSFGGVSVITPGTNTSNQVAVLYQDVNCTEVPVAI
HADQLTPTWRVYSTGSNVFQTRAGCLIGAEHVNNSYECDIPIGAGICASYQTQTNSPR
RARSVASQSIIAYTMSLGAENSVAYSNNSIAIPTNFTISVTTEILPVSMTKTSVDCTM
YICGDSTECSNLLLQYGSFCTQLNRALTGIAVEQDKNTQEVFAQVKQIYKTPPIKDFG
GFNFSQILPDPSKPSKRSFIEDLLFNKVTLADAGFIKQYGDCLGDIAARDLICAQKFN
GLTVLPPLLTDEMIAQYTSALLAGTITSGWTFGAGAALQIPFAMQMAYRFNGIGVTQN
VLYENQKLIANQFNSAIGKIQDSLSSTASALGKLQDVVNQNAQALNTLVKQLSSNFGA
ISSVLNDILSRLDKVEAEVQIDRLITGRLQSLQTYVTQQLIRAAEIRASANLAATKMS
ECVLGQSKRVDFCGKGYHLMSFPQSAPHGVVFLHVTYVPAQEKNFTTAPAICHDGKAH
FPREGVFVSNGTHWFVTQRNFYEPQIITTDNTFVSGNCDVVIGIVNNTVYDPLQPELD
SFKEELDKYFKNHTSPDVDLGDISGINASVVNIQKEIDRLNEVAKNLNESLIDLQELG
KYEQYIKWPWYIWLGFIAGLIAIVMVTIMLCCMTSCCSCLKGCCSCGSCCKFDEDDSE
PVLKGVKLHYT

A polynucleotide sequence for SARS-CoV-2 S polypeptide is provided by SEQ ID NO: 15:

| | |
|---|---|
| 1 | atgtttgttt ttcttgtttt attgccacta gtctctagtc agtgtgttaa tcttacaacc |
| 61 | agaactcaat taccccctgc atacactaat tctttcacac gtggtgttta ttaccctgac |
| 121 | aaagttttca gatcctcagt tttacattca actcaggact tgttcttacc tttcttttcc |
| 181 | aatgttactt ggttccatgc tatacatgtc tctgggacca atggtactaa gaggtttgat |
| 241 | aaccctgtcc taccatttaa tgatggtgtt tattttgctt ccactgagaa gtctaacata |
| 301 | ataagaggct ggatttttgg tactacttta gattcgaaga cccagtccct acttattgtt |
| 361 | aataacgcta ctaatgttgt tattaaagtc tgtgaatttc aattttgtaa tgatccattt |
| 421 | ttgggtgttt attaccacaa aaacaacaaa agttggatgg aaagtgagtt cagagtttat |
| 481 | tctagtgcga ataattgcac ttttgaatat gtctctcagc cttttcttat ggaccttgaa |
| 541 | ggaaaacagg gtaatttcaa aaatcttagg gaatttgtgt ttaagaatat tgatggttat |
| 601 | tttaaaatat attctaagca cacgcctatt aatttagtgc gtgatctccc tcagggtttt |
| 661 | tcggctttag aaccattggt agatttgcca ataggtatta acatcactag gtttcaaact |
| 721 | ttacttgctt tacatagaag ttatttgact cctggtgatt cttcttcagg ttggacagct |
| 781 | ggtgctgcag cttattatgt gggttatctt caacctagga cttttctatt aaaatataat |
| 841 | gaaaatggaa ccattacaga tgctgtagac tgtgcacttg accctctctc agaaacaaag |
| 901 | tgtacgttga aatccttcac tgtagaaaaa ggaatctatc aaacttctaa ctttagagtc |
| 961 | caaccaacag aatctattgt tagatttcct aatattacaa acttgtgccc ttttggtgaa |
| 1021 | gtttttaacg ccaccagatt tgcatctgtt tatgcttgga acaggaagag aatcagcaac |
| 1081 | tgtgttgctg attattctgt cctatataat tccgcatcat tttccacttt taagtgttat |
| 1141 | ggagtgtctc ctactaaatt aaatgatctc tgctttacta atgtctatgc agattcattt |
| 1201 | gtaattagag gtgatgaagt cagacaaatc gctccagggc aaactggaaa gattgctgat |
| 1261 | tataattata aattaccaga tgattttaca ggctgcgtta tagcttggaa ttctaacaat |
| 1321 | cttgattcta aggttggtgg taattataat tacctgtata gattgtttag gaagtctaat |
| 1381 | ctcaaacctt ttgagagaga tatttcaact gaaatctatc aggccggtag cacaccttgt |
| 1441 | aatggtgttg aaggttttaa ttgttacttt cctttacaat catatggttt ccaacccact |
| 1501 | aatggtgttg gttaccaacc atacagagta gtagtacttt cttttgaact tctacatgca |
| 1561 | ccagcaactg tttgtggacc taaaaagtct actaatttgg ttaaaaacaa atgtgtcaat |
| 1621 | ttcaacttca atggtttaac aggcacaggt gttcttactg agtctaacaa aaagtttctg |
| 1681 | cctttccaac aatttggcag agacattgct gacactactg atgctgtccg tgatccacag |
| 1741 | acacttgaga ttcttgacat tacaccatgt tcttttggtg gtgtcagtgt tataacacca |
| 1801 | ggaacaaata cttctaacca ggttgctgtt ctttatcagg atgttaactg cacagaagtc |
| 1861 | cctgttgcta ttcatgcaga tcaacttact cctacttggc gtgtttattc tacaggttct |
| 1921 | aatgtttttc aaacacgtgc aggctgttta ataggggctg aacatgtcaa caactcatat |
| 1981 | gagtgtgaca tacccattgg tgcaggtata tgcgctagtt atcagactca gactaattct |
| 2041 | cctcggcggg cacgtagtgt agctagtcaa tccatcattg cctacactat gtcacttggt |
| 2101 | gcagaaaatt cagttgctta ctctaataac tctattgcca tacccacaaa ttttactatt |
| 2161 | agtgttacca cagaaattct accagtgtct atgaccaaga catcagtaga ttgtacaatg |
| 2221 | tacatttgtg gtgattcaac tgaatgcagc aatcttttgt tgcaatatgg cagtttttgt |
| 2281 | acacaattaa accgtgcttt aactggaata gctgttgaac aagacaaaaa cacccaagaa |
| 2341 | gtttttgcac aagtcaaaca aatttacaaa acaccaccaa ttaaagattt tggtggtttt |
| 2401 | aatttttcac aaatattacc agatccatca aaaccaagca agaggtcatt tattgaagat |
| 2461 | ctacttttca acaaagtgac acttgcagat gctggcttca tcaaacaata tggtgattgc |
| 2521 | cttggtgata ttgctgctag agacctcatt tgtgcacaaa agtttaacgg ccttactgtt |
| 2581 | ttgccacctt tgctcacaga tgaaatgatt gctcaataca cttctgcact gttagcgggt |
| 2641 | acaatcactt ctggttggac ctttggtgca ggtgctgcat tacaaatacc atttgctatg |
| 2701 | caaatggctt ataggtttaa tggtattgga gttacacaga atgttctcta tgagaaccaa |
| 2761 | aaattgattg ccaaccaatt taatagtgct attggcaaaa ttcaagactc actttcttcc |
| 2821 | acagcaagtg cacttggaaa acttcaagat gtggtcaacc aaaatgcaca agctttaaac |
| 2881 | acgcttgtta aacaacttag ctccaatttt ggtgcaattt caagtgtttt aaatgatatc |
| 2941 | ctttcacgtc ttgacaaagt tgaggctgaa gtgcaaattg ataggttgat cacaggcaga |
| 3001 | cttcaaagtt tgcagacata tgtgactcaa caattaatta gagctgcaga aatcagagct |
| 3061 | tctgctaatc ttgctgctac taaaatgtca gagtgtgtac ttggacaatc aaaaagagtt |
| 3121 | gatttttgtg gaaagggcta tcatcttatg tccttccctc agtcagcacc tcatggtgta |
| 3181 | gtcttcttgc atgtgactta tgtccctgca caagaaaaga acttcacaac tgctcctgcc |
| 3241 | atttgtcatg atggaaaagc acactttcct cgtgaaggtg tctttgtttc aaatggcaca |
| 3301 | cactggtttg taacacaaag gaatttttat gaaccacaaa tcattactac agacaacaca |
| 3361 | tttgtgtctg gtaactgtga tgttgtaata ggaattgtca acaacacagt ttatgatcct |
| 3421 | ttgcaacctg aattagactc attcaaggag gagttagata aatattttaa gaatcataca |
| 3481 | tcaccagatg ttgatttagg tgacatctct ggcattaatg cttcagttgt aaacattcaa |
| 3541 | aaagaaattg accgcctcaa tgaggttgcc aagaatttaa atgaatctct catcgatctc |
| 3601 | caagaacttg gaaagtatga gcagtatata aaatggccat ggtacatttg gctaggtttt |
| 3661 | atagctggct tgattgccat agtaatggtg acaattatgc tttgctgtat gaccagttgc |
| 3721 | tgtagttgtc tcaagggctg ttgttcttgt ggatcctgct gcaaatttga tgaagacgac |
| 3781 | tctgagccag tgctcaaagg agtcaaatta cattacacat aa |

In some embodiments, a target protein antigen is or comprises a SARS-CoV-2 S polypeptide. In some embodiments, a target protein antigen comprises a polypeptide having at least 80%, at least 85%, at least 90%, at least 91%, at least 92%, at least 93%, at least 94%, at least 95%, at least 96%, at least 97%, at least 98%, at least 99% or 100% identity to an amino acid sequence of a SARS-CoV-2 S polypeptide or a fragment thereof as described herein. In some embodiments, a target protein antigen comprises a polypeptide having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 14.

In some embodiments, an antigen fragment comprises an epitope of a SARS-CoV-2 S polypeptide. In some embodiments, an antigen fragment comprises a polypeptide having at least 80%, at least 85%, at least 90%, at least 91%, at least 92%, at least 93%, at least 94%, at least 95%, at least 96%, at least 97%, at least 98%, at least 99% or 100% identity to an amino acid sequence of a SARS-CoV-2 S polypeptide or a fragment thereof as described herein. In some embodiments, an antigen fragment comprises a polypeptide having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 14.

In some embodiments, an antigen fragment comprises an epitope of a SARS-CoV-2 S polypeptide. In some embodiments, an antigen fragment comprises a polynucleotide encoding a polypeptide having at least 80%, at least 85%, at least 90%, at least 91%, at least 92%, at least 93%, at least 94%, at least 95%, at least 96%, at least 97%, at least 98%, at least 99% or 100% identity to an amino acid sequence of a SARS-CoV-2 S polypeptide or a fragment thereof as described herein. In some embodiments, an antigen fragment comprises a polynucleotide having at least 80%, at least 85%, at least 90%, at least 91%, at least 92%, at least 93%, at least 94%, at least 95%, at least 96%, at least 97%, at least 98%, at least 99% or 100% identity to the nucleic acid sequence of SEQ ID NO: 15. Due to degeneracy in the genetic code, those of ordinary skill in the art would understand that other DNA sequences (including codon-optimized sequences) could encode these polypeptides, as well as the others disclosed herein.

### Adjuvants or Immunomodulators

In some embodiments, an adjuvant (also referred to as an immunomodulator) disclosed herein can be used to elicit and/or modulate an immune response elicited by an antigen (e.g., antigen fragment, antigen variant, or antigen fragment variant) described herein. In some embodiments, an adjuvant disclosed herein comprises a complement binding polypeptide. In some embodiments, a complement binding polypeptide comprises a complement C3d binding polypeptide. An exemplary C3d binding polypeptide is an immunoglobulin-binding protein (Sbi) of Staphylococcus aureus.

In some embodiments, a complement binding polypeptide described herein can be an adjuvant or function as an adjuvant. In some embodiments, a complement binding polypeptide comprises a complement C3d binding polypeptide. An exemplary C3d binding polypeptide is an immunoglobulin-binding protein (Sbi) of Staphylococcus aureus.

In some embodiments of any of the fusion polypeptides, polynucleotides encoding fusion polypeptides, compositions, methods or uses disclosed herein, an adjuvant disclosed herein can be used alone or in combination with an antigen disclosed herein to modulate and/or enhance an immune response. In an embodiment, an adjuvant disclosed herein can comprise a fusion polypeptide which comprises an antigen, e.g., an antigen fragment, an antigen variant, or an antigen fragment variant, and an adjuvant (e.g., a C3d binding polypeptide). In some embodiments, also disclosed herein are polynucleotides encoding fusion polypeptides comprising an antigen, e.g., an antigen fragment, an antigen variant, or an antigen fragment variant, and an adjuvant (e.g., C3d binding polypeptide). In some embodiments, a polynucleotide comprises DNA or RNA. In some embodiments, a polynucleotide comprises RNA, e.g., messenger RNA.

S. aureus Binder of Immunoglobulin (Sbi)

As disclosed herein, S. aureus binder of immunoglobulin (Sbi) is an exemplary polypeptide that can activate the alternative complement pathway and modulate B cell activity. The alternative complement pathway is known in the field and described in e.g., Maillard N. et al. (2015), “Current Understanding of the Role of Complement in IgA Nephropathy” J Am Soc Nephrol., vol. 26(7), pp. 1503-12, the entire contents of which are hereby incorporated by reference. The alternate complement pathway comprises a series of proteolytic cascades starting from cleavage of complement protein C3 into C3b and C3a. C3b can in turn be cleaved into fragments including iC3b, C3f, C3c, and C3dg. The C3dg fragment can be further cleaved into C3g and C3d. The C3d fragment, among other fragments in the alternative complement pathway, has opsonization activity and can bind to complement receptor 2 (CR2, also known as CD21) on B cells allowing engagement of CR2 with the B cell antigen receptor (BCR), thus lowering the threshold for B cell activation (see, e.g., Yang Y. et al., (2019) “Utilization of Staphylococcal Immune Evasion Protein Sbi as a Novel Vaccine Adjuvant,” Front. Immunol., 9:3139. doi: 10.3389/fimmu.2018.03139. PMID: 30687332; PMCID: PMC6336717).

Sbi can bind complement protein C3d (as described in Clark et al. (2011) Mol Immunol. 48(4): 452-462, the entire contents of which is incorporated herein by reference). Sbi comprises two immunoglobulin binding domains (Domains I and II) and two complement C3d binding domains (Domains III and IV). Sbi domains III and IV can bind C3d (in native C3, iC3b and C3dg) and can result in fluid phase consumption of C3 via activation of the alternative complement pathway (see Clark et al 2011). It has also been shown that Sbi can be secreted and is involved in S. aureus immune evasion (Burman et al., 2008 J. Biol. Chem; 283:17579-17593). Furthermore, as described in Yang 2019, Sbi can also bind to Factor H Related (“FHR”) proteins.

Without wishing to be bound by any particular theory, it is believed that in some embodiments, a complement C3d-binding polypeptide from Sbi of S. aureus can be used as an adjuvant to enhance and/or modulate an immune response from an antigen described herein. In some embodiments, the immune response is elicited by an antigen, e.g., an antigen fragment, antigen variant or antigen fragment variant, disclosed herein. In some embodiments, the immune response is elicited by a component of Sbi of S. aureus. In some embodiments, a complement C3d-binding polypeptide from Sbi of S. aureus can activate B cells by binding to C3d. Further without wishing to be bound by any particular theory, it is believed that in some embodiments, a complement C3d-binding polypeptide in a fusion polypeptide comprising a complement C3d-binding polypeptide and an antigen (e.g., as described herein), enhances an immune response from the antigen by binding to C3d and modulating (1) opsonization of the antigen by C3d, (2) binding of C3d to CR2, and (3) engagement of CR2 with a B cell receptor (e.g., clustering of CR2 and BCR). In some embodiments, this reduces the threshold for B cell activation and enhances the immune response from the antigen. In some embodiments, a complement C3d-binding polypeptide from Sbi of S. aureus can activate B cells by binding to Factor H Related (“FHR”) proteins.

S. aureus Sbi is referenced by Gene ID: 3919725 and NCBI Ref No.: NC_007795.1. A polynucleotide sequence of Sbi is provided herein as SEQ ID NO: 16:

| | |
|---|---|
| 1 | atgaaaaata aatatatctc gaagttgcta gttggggcag caacaattac gttagctaca |
| 61 | atgatttcaa atggggaagc aaaagcgagt gaaaacacgc aacaaacttc aactaagcac |
| 121 | caaacaactc aaaacaacta cgtaacagat caacaaaaag ctttttatca agtattacat |
| 181 | ctaaaaggta tcacagaaga acaacgtaac caatacatca aaacattacg cgaacaccca |
| 241 | gaacgtgcac aagaagtatt ctctgaatca cttaaagaca gcaagaaccc agaccgacgt |
| 301 | gttgcacaac aaaacgcttt ttacaatgtt cttaaaaatg ataacttaac tgaacaagaa |
| 361 | aaaaataatt acattgcaca aattaaagaa aaccctgata gaagccaaca agtttgggta |
| 421 | gaatcagtac aatcttctaa agctaaagaa cgtcaaaata ttgaaaatgc ggataaagca |
| 481 | attaaagatt tccaagataa caaagcacca cacgataaat cagcagcata tgaagctaac |
| 541 | tcaaaattac ctaaagattt acgtgataaa aacaaccgct ttgtagaaaa agtttcaatt |
| 601 | gaaaaagcaa tcgttcgtca tgatgagcgt gtgaaatcag caaatgatgc aatctcaaaa |
| 661 | ttaaatgaaa aagattcaat tgaaaacaga cgtttagcac aacgtgaagt taacaaagca |
| 721 | cctatggatg taaaagagca tttacagaaa caattagacg cattagttgc tcaaaaagat |
| 781 | gctgaaaaga aagtggcgcc aaaagttgag gctcctcaaa ttcaatcacc acaaattgaa |
| 841 | aaacctaaag tagaatcacc aaaagttgaa gtccctcaaa ttcaatcacc aaaagttgag |
| 901 | gttcctcaat ctaaattatt aggttactac caatcattaa aagattcatt taactatggt |
| 961 | tacaagtatt taacagatac ttataaaagc tataaagaaa aatatgatac agcaaagtac |
| 1021 | tactataata cgtactataa atacaaaggt gcgattgatc aaacagtatt aacagtacta |
| 1081 | ggtagtggtt ctaaatctta catccaacca ttgaaagttg atgataaaaa cggctactta |
| 1141 | gctaaatcat atgcacaagt aagaaactat gtaactgagt caatcaatac tggtaaagta |
| 1201 | ttatatactt tctaccaaaa cccaacatta gtaaaaacag ctattaaagc tcaagaaact |
| 1261 | gcatcatcaa tcaaaaatac attaagtaat ttattatcat tctggaaata a |

A polypeptide sequence of Sbi is provided herein as SEQ ID NO: 17.

MKNKYISKLLVGAATITLATMISNGEAKASENTQQTSTKHQTTQNNYVT
DQQKAFYQVLHLKGITEEQRNQYIKTLREHPERAQEVFSESLKDSKNPD
RRVAQQNAFYNVLKNDNLTEQEKNNYIAQIKENPDRSQQVWVESVQSSK
AKERQNIENADKAIKDFQDNKAPHDKSAAYEANSKLPKDLRDKNNRFVE
KVSIEKAIVRHDERVKSANDAISKLNEKDSIENRRLAQREVNKAPMDVK
EHLQKQLDALVAQKDAEKKVAPKVEAPQIQSPQIEKPKVESPKVEVPQI
QSPKVEVPQSKLLGYYQSLKDSFNYGYKYLTDTYKSYKEKYDTAKYYYN
TYYKYKGAIDQTVLTVLGSGSKSYIQPLKVDDKNGYLAKSYAQVRNYVT
ESINTGKVLYTFYQNPTLVKTAIKAQETASSIKNTLSNLLSFWK

In some embodiments, any of the fusion polypeptides, fusion nucleotides, compositions, methods or uses disclosed herein, comprises a complement C3d-binding polypeptide of Sbi of S. aureus. In some embodiments, a Sbi complement C3d binding polypeptide comprises one or both of domain III and domain IV of Sbi, or a functional fragment or a variant thereof.

In some embodiments, a Sbi complement C3d binding polypeptide comprises an amino acid sequence having at least 80%, at least 85%, at least 90%, at least 91%, at least 92%, at least 93%, at least 94%, at least 95%, at least 96%, at least 97%, or 100% identity to the amino acid sequence of SEQ ID NO: 3. In some embodiments, a Sbi complement C3d binding polypeptide comprises an amino acid sequence having at least 80%, at least 85%, at least 90%, at least 91%, at least 92%, at least 93%, at least 94%, at least 95%, at least 96%, at least 97%, or 100% identity to the amino acid sequence of SEQ ID NO: 4. In some embodiments, a Sbi complement C3d binding polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 17.

In some embodiments, a Sbi complement C3d binding polypeptide is encoded by a polynucleotide which encodes an amino acid having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 3. In some embodiments, a Sbi complement C3d binding polypeptide is encoded by a polynucleotide which encodes an amino acid having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 4. In some embodiments, a Sbi complement C3d binding polypeptide is encoded by a polynucleotide which encodes an amino acid having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 17. Due to degeneracy in the genetic code, those of ordinary skill in the art would understand that other DNA sequences (including codon-optimized sequences) could encode these polypeptides, as well as the others disclosed herein.

### Fusion Polypeptides

This disclosure provides fusion polypeptides comprising (a) an antigen (e.g., an antigen variant, an antigen fragment or an antigen fragment variant); and (b) a complement C3d-binding polypeptide from an immunoglobulin-binding protein (Sbi) of Staphylococcus aureus. Exemplary fusion polypeptides disclosed herein are provided in Tables 1 and 2.

In some embodiments, an antigen, e.g., an antigen fragment, antigen variant or antigen fragment variant, has an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 2.

In some embodiments, an antigen, e.g., an antigen fragment, antigen variant or antigen fragment variant, has an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 7.

In some embodiments, a complement C3d-binding polypeptide from an immunoglobulin-binding protein (Sbi) of Staphylococcus aureus comprises an Sbi domain III. In some embodiments, an Sbi domain III comprises an amino acid sequence with at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 3.

In some embodiments, a complement C3d-binding polypeptide from an immunoglobulin-binding protein (Sbi) of Staphylococcus aureus comprises an Sbi domain IV. In some embodiments, an Sbi domain IV comprises an amino acid sequence with at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 4.

In some embodiments, a complement C3d-binding polypeptide from an immunoglobulin-binding protein (Sbi) of Staphylococcus aureus comprises an Sbi domain III and an Sbi domain IV. In some embodiments, a complement C3d-binding polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 5.

In some embodiments, (a) and (b) are contiguous or separated by a linker. In some embodiments, a linker is a peptidyl linker. In some embodiments, a peptidyl linker comprises at least 60% glycine and/or serine. In some embodiments, a linker is a Gly-Gly-Gly-Gly-Ser (Gly4-Ser) linker or a histidine linker. In some embodiments, a linker is a Gly4-Ser linker. In some embodiments, a linker comprises 1, 2, 3, 4, 5, 6, 7, 8, 9, or 10 repeats of the Gly4-Ser linker. In some embodiments, a linker comprises 3 repeats of the Gly4-Ser linker. In some embodiments, a linker comprises the sequence of SEQ ID NO: 6.

In some embodiments, a polypeptide further comprises a secretion peptide. In some embodiments, a secretion peptide is about 10-30 amino acids in length. In some embodiments, a secretion peptide is about 10, 11, 12, 13, 14, 15, 16, 17, 18, 19, 20, 21, 22, 23, 24, 25, 26, 27, 28, 29 or 30 amino acids in length. In some embodiments, a secretion peptide comprises an amino acid having at least 80%, 85%, 90%, or 100% identity to the amino acid sequence of SEQ ID NO: 1. In some embodiments, a secretion peptide comprises an amino acid having at least 80%, at least 85%, at least 90%, or at least 100% identity to the amino acid sequence of SEQ ID NO: 20.

In some embodiments, a polypeptide has at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 8.

In some embodiments, a polynucleotide encodes a fusion polypeptide having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 8.

TABLE 1
Exemplary amino acid sequences of components of polypeptide fusions disclosed herein.

| SEQ ID | | |
|---|---|---|
| NO | Feature | Amino acid sequence |
| 1 | SARS-CoV-2 Spike residues 1-13 | MFVFLVLLPLVSS |
| | (secretion peptide) | |
| | Also referred to as S(ss) | |
| 20 | SARS-CoV-2 Spike residues 1-13 | MFVFLVLLPLVSSAA |
| | (secretion peptide) with AA | |
| | dipeptide | |
| 2 | SARS-CoV-2 Spike residues 331- | NITNLCPFGEVFNATRFASVYAWNRKRISNCVADYSVLYNSASFSTFKC |
| | 527 (RBD domain) | YGVSPTKLNDLCFTNVYADSFVIRGDEVRQIAPGQTGKIADYNYKLPD |
| | | DFTGCVIAWNSNNLDSKVGGNYNYLYRLFRKSNLKPFERDISTEIYQA |
| | | GSTPCNGVEGFNCYFPLQSYGFQPTNGVGYQPYRVVVLSFELLHAPAT |
| | | VCGP |
| 3 | Domain III of Sbi from S. aureus | IENADKAIKDFQDNKAPHDKSAAYEANSKLPKDLRDKNNRFV |
| | strain Mu50 | |
| 4 | Domain IV of Sbi from S. aureus | EKVSIEKAIVRHDERVKSANDAISKLNEKDSIENRRLAQREVNKAPMD |
| | strain Mu50 | VKEHLQKQLD |
| 5 | Domains III and IV of Sbi from | IENADKAIKDFQDNKAPHDKSAAYEANSKLPKDLRDKNNRFVEKVSIE |
| | S. aureus strain Mu50 | KAIVRHDERVKSANDAISKLNEKDSIENRRLAQREVNKAPMDVKEHLQ |
| | | KQLD |
| 6 | Gly₄ Ser linker | GGGGSGGGGSGGGGS |

TABLE 2
Exemplary amino acid sequences of polypeptide fusions encoded by polynucleotide sequences
disclosed herein.

| SEQ ID | | |
|---|---|---|
| NO | Feature | Amino acid sequence |
| 7 | S(ss)-RBD with ″AA″ linker, | MFVFLVLLPLVSSAANITNLCPFGEVFNATRFASVYAWNRKRISNCVAD |
| | referred to herein as S(ss)-RBD- | YSVLYNSASFSTFKCYGVSPTKLNDLCFTNVYADSFVIRGDEVRQIAPG |
| | AA | QTGKIADYNYKLPDDFTGCVIAWNSNNLDSKVGGNYNYLYRLFRKSN |
| | | LKPFERDISTEIYQAGSTPCNGVEGFNCYFPLQSYGFQPTNGVGYQPY |
| | | RVVVLSFELLHAPATVCGP |
| 8 | S(ss)-RBD with ″AA″ linker joined | MFVFLVLLPLVSSAANITNLCPFGEVFNATRFASVYAWNRKRISNCVAD |
| | (e.g., directly or indirectly) to | YSVLYNSASFSTFKCYGVSPTKLNDLCFTNVYADSFVIRGDEVRQIAPG |
| | Sbi domains III and IV with a | QTGKIADYNYKLPDDFTGCVIAWNSNNLDSKVGGNYNYLYRLFRKSN |
| | (Gly₄Ser)3 linker, referred to | LKPFERDISTEIYQAGSTPCNGVEGFNCYFPLQSYGFQPTNGVGYQPY |
| | herein as | RVVVLSFELLHAPATVCGPGGGGSGGGGSGGGGSIENADKAIKDFQD |
| | S(ss)-RBD-AA-Sbi(III-IV) | NKAPHDKSAAYEANSKLPKDLRDKNNRFVEKVSIEKAIVRHDERVKSA |
| | | NDAISKLNEKDSIENRRLAQREVNKAPMDVKEHLQKQLD |

### Polynucleotides Encoding Fusion Polypeptides

Among other things, the disclosure provides polynucleotides (e.g., comprising nucleotide sequences) encoding fusion polypeptides comprising (a) one or more antigens (e.g., one or more antigen variants, one or more antigen fragments or one or more antigen fragment variants); and (b) a complement C3d-binding polypeptide from an immunoglobulin-binding protein (Sbi) of Staphylococcus aureus. In some embodiments, a polynucleotide comprises a nucleotide sequence encoding a fusion polypeptide comprising: (a) one or more antigens (e.g., one or more antigen variants, one or more antigen fragments or one or more antigen fragment variants); and (b) a complement C3d-binding polypeptide from an immunoglobulin-binding protein (Sbi) of Staphylococcus aureus. In some embodiments, a polynucleotide comprises: a modified nucleobase, a modified deoxyribose, a modified ribose, a modified backbone, or any combination thereof. Exemplary polynucleotide sequences are provided in Table 3.

In some embodiments, (a) is disposed C-terminus of (b).

In some embodiments, (a) is disposed N-terminus of (b).

In some embodiments, (a) and (b) are contiguous. In some embodiments, (a) and (b) are separated by a nucleotide sequence encoding a linker. In some embodiments, the linker is or comprises a peptidyl linker. In some embodiments, the peptidyl linker comprises at least 60% glycine and/or serine. In some embodiments, a linker is or comprises a glycine-serine linker. In some embodiments, a linker is or comprises a Gly-Gly-Gly-Gly-Ser (Gly4-Ser) linker or a histidine linker.

In some embodiments, a polynucleotide further comprises a nucleotide sequence encoding a secretion peptide. In some embodiments, a polynucleotide disclosed herein further comprises a nucleotide sequence encoding a polymerization domain. In some embodiments, a polynucleotide disclosed herein further comprises a nucleotide sequence encoding a trafficking domain. In some embodiments, a trafficking domain directs a polypeptide it is associated with to an MHC molecule (e.g., MHC class I and/or class II molecule). In some embodiments, a polynucleotide disclosed herein further comprises a nucleotide sequence encoding a transmembrane domain.

In some embodiments, a fusion polypeptide described herein further comprises a secretion peptide. In some embodiments, a fusion polypeptide described herein further comprises a polymerization domain. In some embodiments, a fusion polypeptide described herein further comprises a trafficking domain. In some embodiments, a trafficking domain directs a polypeptide it is associated with to an MHC molecule (e.g., MHC class I and/or class II molecule). In some embodiments, a fusion polypeptide described herein further comprises a transmembrane domain.

In some embodiments, a polynucleotide is or comprises RNA.

In some embodiments, a polynucleotide is or comprises DNA.

In some embodiments, a polynucleotide encodes an antigen (e.g., an antigen fragment, an antigen variant or an antigen fragment variant) which is characterized in that when expressed in vivo, it binds to a Major Histocompatibility Complex (MHC) molecule. In some embodiments, an MHC molecule is or comprises an MHC I molecule (e.g., HLA-I molecule) or an MHC II molecule (e.g., HLA-II molecule).

In some embodiments, a polynucleotide encodes an antigen (e.g., an antigen fragment, an antigen variant or an antigen fragment variant) which is characterized in that when expressed in vivo, it does not bind to an MHC molecule.

In some embodiments, a polynucleotide encodes an antigen (e.g., an antigen fragment, an antigen variant or an antigen fragment variant) which is characterized in that when expressed in vivo, it folds into a three-dimensional conformation that is substantially identical to the three-dimensional conformation of the antigen, e.g., an antigen fragment, an antigen variant or an antigen fragment variant, as it is in its native position in the target protein antigen.

In some embodiments, a polynucleotide encoding an antigen, e.g., an antigen fragment, antigen variant or antigen fragment variant, further comprises an amino acid sequence from a second target protein antigen.

In some embodiments, a polynucleotide has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 9.

In some embodiments, a polynucleotide has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 10.

In some embodiments, a polynucleotide has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 18.

In some embodiments, a polynucleotide has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 19.

TABLE 3
Exemplary polynucleotide sequences disclosed herein.

| SEQ ID | | Nucleic acid sequence |
|---|---|---|
| NO | Feature | |
| 9 | S(ss)-RBD with | CTTGTTCTTTTTGCAGAAGCTCAGAATAAACGCTCAACTTTGGCCACCatgttcgtattcctcgt |
| | ″AA″ linker, | cttgcttcctctcgtctccagtgcagcaaacatcaccaacctctgcccttttggcgaagtgttcaacg |
| | referred to herein | ccacacgctttgcctctgtgtacgcatggaataggaagagaatatctaactgcgttgccgattattct |
| | as S(ss)-RBD-AA | gtactctacaatagtgcatcattttccacgttcaagtgctatggggtgtctccaaccaaattgaacga |
| | | tctgtgcttcaccaatgtgtatgcagatagctttgtaattagaggggacgaagtgcggcaaattgccc |
| | | ctggtcaaacgggcaaaatcgcagattataattataaactcccagatgattttactggctgcgtaatc |
| | | gcatggaactcaaacaatcttgactctaaggtggggggaactataactatctctacagactgtttcga |
| | | aagtccaatctgaagccgtttgagcgggacatttccactgagatctatcaagccgggtccaccccgtg |
| | | caacggggtggaaggctttaactgttactttcctcttcagagttatgggttccaaccgactaatggtg |
| | | taggctatcagccataccgagtcgttgttctcagctttgagctgctgcatgcacctgctacagtttgc |
| | | ggcccaTAATGATAGACCAGCCTCAAGAACACCCGAATGGAGTCTCTAAGCTACATAATACCAACTTA |
| | | CACTTTACAAAATGTTGTCCCCCAAAATGTAGCCATTCGTATCTGCTCCTAATAAAAAGAAAGTTTCT |
| | | TCACATTCT |
| 10 | S(ss)-RBD with | CTTGTTCTTTTTGCAGAAGCTCAGAATAAACGCTCAACTTTGGCCACCatgttcgtattcctcgt |
| | ″AA″ linker joined | cttgcttcctctcgtctccagtgcagcaaacatcaccaacctctgcccttttggcgaagtgttcaacg |
| | (e.g., directly or | ccacacgctttgcctctgtgtacgcatggaataggaagagaatatctaactgcgttgccgattattct |
| | indirectly) to Sbi | gtactctacaatagtgcatcattttccacgttcaagtgctatggggtgtctccaaccaaattgaacga |
| | domains III and | tctgtgcttcaccaatgtgtatgcagatagctttgtaattagaggggacgaagtgcggcaaattgccc |
| | IV with a | ctggtcaaacgggcaaaatcgcagattataattataaactcccagatgattttactggctgcgtaatc |
| | (Gly₄Ser)3 linker, | gcatggaactcaaacaatcttgactctaaggtggggggaactataactatctctacagactgtttcga |
| | referred to herein | aagtccaatctgaagccgtttgagcgggacatttccactgagatctatcaagccgggtccaccccgtg |
| | as S(ss)-RBD-AA- | caacggggtggaaggctttaactgttactttcctcttcagagttatgggttccaaccgactaatggtg |
| | Sbi(III-IV) | taggctatcagccataccgagtcgttgttctcagctttgagctgctgcatgcacctgctacagtttgc |
| | | ggcccagggggtggaggatccggtggtggtggctctggaggaggaggcagcatagagaatgccgataa |
| | | agctatcaaggactttcaggacaacaaagcaccgcacgataagagcgctgcttacgaggccaattcaa |
| | | aattgccaaaagacctccgagacaagaacaaccggttcgttgagaaggttagtattgaaaaggccatt |
| | | gtgcgccatgacgaacgcgtcaagtctgccaacgatgctattagtaagctgaacgaaaaggattctat |
| | | cgagaaccgaaggcttgctcaaagagaggttaacaaggcccctatggacgttaaagaacatttgcaaa |
| | | agcaattggatTAATGATAGACCAGCCTCAAGAACACCCGAATGGAGTCTCTAAGCTACATAATACCA |
| | | ACTTACACTTTACAAAATGTTGTCCCCCAAAATGTAGCCATTCGTATCTGCTCCTAATAAAAAGAAAG |
| | | TTTCTTCACATTCT |
| 18 | Domain III of Sbi | atagagaatgccgataaagctatcaaggactttcaggacaacaaagcaccgcacgataagagcgctgc |
| | from S. aureus | ttacgaggccaattcaaaattgccaaaagacctccgagacaagaacaaccggttcgtt |
| | strain Mu50 | |
| 19 | Domain IV of Sbi | gagaaggttagtattgaaaaggccattgtgcgccatgacgaacgcgtcaagtctgccaacgatgcta |
| | from S. aureus | ttagtaagctgaacgaaaaggattctatcgagaaccgaaggcttgctcaaagagaggttaacaaggcc |
| | strain Mu50 | cctatggacgttaaagaacatttgcaaaagcaattggat |

Modified Ribonucleotides: Ac4C and 5-hmU

Polynucleotides disclosed herein can be a polyribonucleotide which further comprises one or more modified ribonucleotides comprising: a modified nucleobase, a modified ribose, a modified backbone, or any combination thereof.

In some embodiments, a polynucleotide is a polyribonucleotide. In some embodiments, a polyribonucleotide comprises one or more modified ribonucleotides. In some embodiments, a polyribonucleotide comprises a modified nucleobase, a modified ribose, a modified backbone, or any combination thereof.

In some embodiments, a polyribonucleotide comprises one or more modified nucleosides or nucleobases.

In some embodiments, a modified ribonucleotides comprises an N4-acetylcytidine nucleoside. In some embodiments, the nucleoside has the following structure:

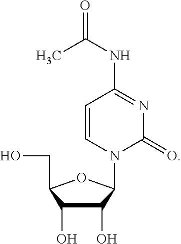

In some embodiments, a modified ribonucleotide comprises a 5-hydroxymethyluridine nucleoside. In some embodiments, the nucleoside has the following structure:

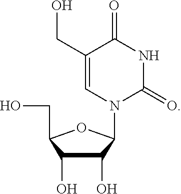

In some embodiments, a modified ribonucleotide has a structure provided in Table 3A. In some embodiments, a modified ribonucleotide has a structure provided in Table 3B when incorporated in a polynucleotide. In some embodiments, a polynucleotide (e.g., a polyribonucleotide) comprises one or more modified ribonucleotides that have a structure provided in Table 3B.

TABLE 3A
Exemplary modified ribonucleotides
N4-acetylcytidine:

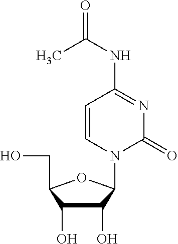

N4-acetylcytidine monophosphate:

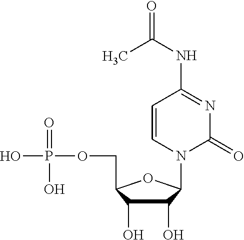

N4-acetylcytidine diphosphate:

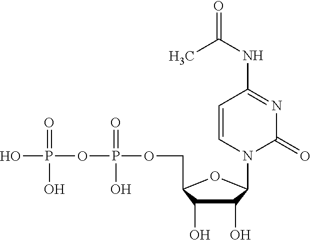

N4-acetylcytidine triphosphate:

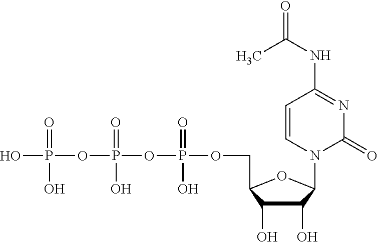

5-hydroxymethyluridine:

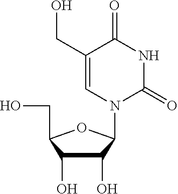

5-hydroxymethyluridine
monophosphate:

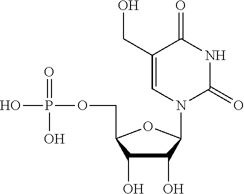

5-hydroxymethyluridine
diphosphate:

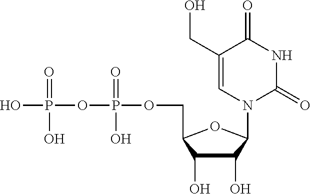

5-hydroxymethyluridine triphosphate:

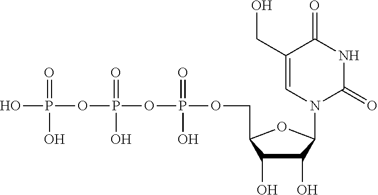

TABLE 3B
Exemplary modified ribonucleotides (when incorporated
into a polyribonucleotide)
N4-acetylcytidine:

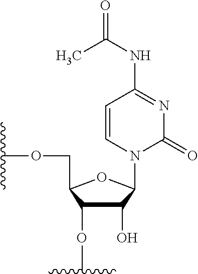

N4-acetylcytidine monophosphate:

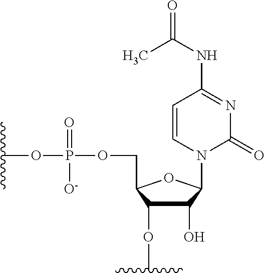

N4-acetylcytidine diphosphate:

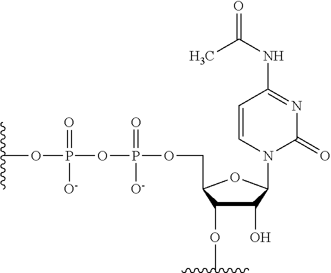

N4-acetylcytidine triphosphate:

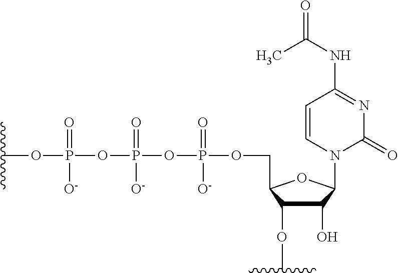

5-hydroxymethyluridine:

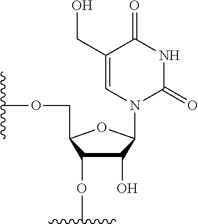

5-hydroxymethyluridine
monophosphate:

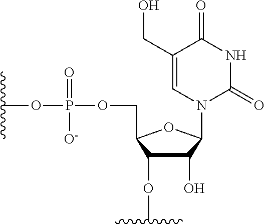

5-hydroxymethyluridine
diphosphate:

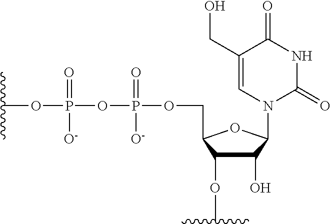

5-hydroxymethyluridine triphosphate:

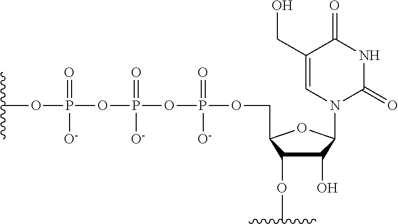

Note: [symbol]
indicates the position of attachment to an adjacent ribonucleotide.

In some embodiments, a polyribonucleotide comprises cytidine nucleosides. In some embodiments, at least 5% of cytidine nucleosides in a polyribonucleotide comprise N4-acetylcytidine. In some embodiments, less than 100% of cytidine nucleosides in a polyribonucleotide comprise N4-acetylcytidine. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of cytidine nucleosides in a polyribonucleotide comprise N4-acetylcytidine.

In some embodiments, a polyribonucleotide comprises uridine nucleosides. In some embodiments, at least 5% of uridine residues in a polyribonucleotide comprise 5-hydroxymethyluridine. In some embodiments, less than 100% of uridine nucleosides in a polyribonucleotide comprise 5-hydroxymethyluridine. In some embodiments, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of uridine nucleosides in a polyribonucleotide comprise 5-hydroxymethyluridine. In some embodiments, more than 60% of uridine nucleosides in a polyribonucleotide comprise 5-hydroxymethyluridine.

In some embodiments, a polyribonucleotide disclosed herein comprises one or more modified ribonucleotides comprises a nucleoside comprising: N4-acetylcytidine, 5-hydroxymethyluridine, N1-methylpseudouridine, pyridin-4-one ribonucleoside, 5-aza-uridine, 6-aza-uridine, 2-thio-5-aza-uridine, 2-thio-uridine (s2U), 5-methyl cytidine (m5C), 5-aza-cytidine, 6-aza-cytidine, pseudoisocytidine, 3-methyl-cytidine (m3C), 5-formyl-cytidine (f5C), N4-methyl-cytidine (m4C), 2-amino-purine, 2, 6-diaminopurine, 2-amino-6-halo-purine, 6-halo-purine, inosine (I), 1-methyl-inosine (ml I), wyosine (imG), methylwyosine (mimG), 5-hydroxycytidine, 5-hydroxymethylcytidine, 5-carboxycytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N1-hydroxypseudouridine, 2′-O-methyl-N1-methylpseudouridine, N1-ethylpseudouridine, N1-hydroxymethylpseudouridine, ara-uridine, N6-methyladenosine, 2-aminoadenosine, 3-methyladenosine, 7-deazaadenosine, 8-oxoadenosine, thienoguanosine, 7-deazaguanosine, 8-oxoguanosine, 6-O-methylguanine, or any combination thereof.

### Modified Ribose

Polynucleotides disclosed herein can be a polyribonucleotide which further comprises one or more modified ribonucleotides comprising: a modified nucleobase, a modified ribose, a modified backbone, or any combination thereof.

In some embodiments, a polynucleotide is a polyribonucleotide. In some embodiments, a polyribonucleotide comprises one or more modified ribonucleotides. In some embodiments, a modified ribonucleotide comprises a modified ribose.

In some embodiments, a modified ribonucleotide comprises a 2′-O-acetylated ribose. In some embodiments, a modified ribonucleotide comprises a structure of:

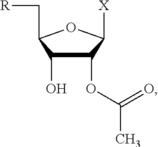

(a) wherein R is a monophosphate, a diphosphate, or a triphosphate; and
(b) wherein X is an adenine nucleobase, a guanine nucleobase, a cytosine nucleobase (e.g., N4-acetylcytosine), or a uracil nucleobase (e.g., 5-hydroxymethyluracil).

In some embodiments, a modified ribonucleotide comprises a 2′-O-acetylated ribose and an adenine nucleobase.

In some embodiments, a modified ribonucleotide comprises a 2′-O-acetylated ribose and a guanine nucleobase.

In some embodiments, a modified ribonucleotide comprises a 2′-O-acetylated ribose and a cytosine nucleobase.

In some embodiments, a modified ribonucleotide comprises a 2′-O-acetylated ribose and a N4-acetylcytidine nucleoside.

In some embodiments, a modified ribonucleotide comprises a 2′-O-acetylated ribose and a uracil nucleobase.

In some embodiments, a modified ribonucleotide comprises a 2′-O-acetylated ribose and a 5-hydroxymethyluridine nucleoside.

In some embodiments, a modified ribonucleotide comprises a 2′-O-acetylated ribose and a N1-methylpseudouracil nucleobase.

In some embodiments, a modified ribonucleotide comprises a structure provided in Table 3C. In some embodiments, a modified ribonucleotide comprises a structure provided in Table 3D when incorporated in a polynucleotide. In some embodiments, a polynucleotide (e.g., a polyribonucleotide) comprises one or more modified ribonucleotides that have a structure provided in Table 3D.

TABLE 3C
Exemplary modified ribonucleotides
2′-O-acetyl N4-acetylcytidine:

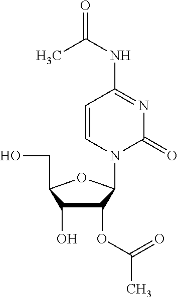

2′-O-acetyl N4-acetylcytidine
monophosphate:

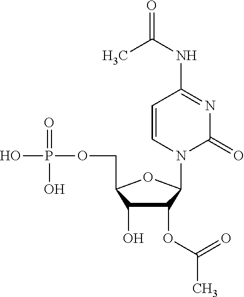

2′-O-acetyl N4-acetylcytidine
diphosphate:

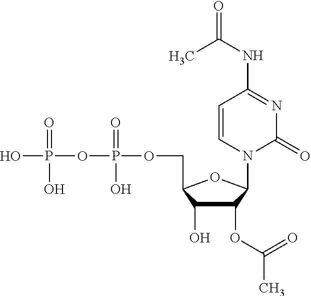

2′-O-acetyl N4-acetylcytidine
triphosphate:

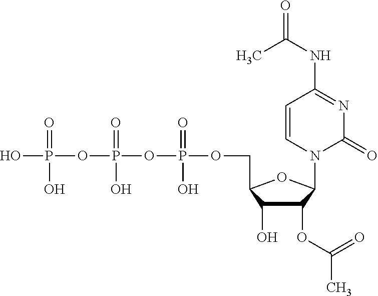

2′-O-acetyl 5-
hydroxymethyluridine:

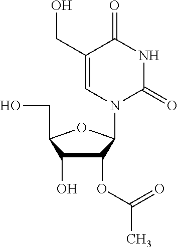

2′-O-acetyl 5-hydroxymethyluridine
monophosphate:

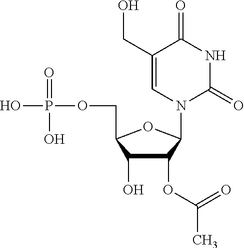

2′-O-acetyl 5-
hydroxymethyluridine
diphosphate:

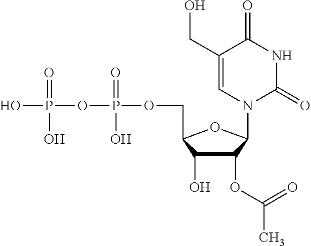

2′-O-acetyl 5-hydroxymethyluridine
triphosphate:

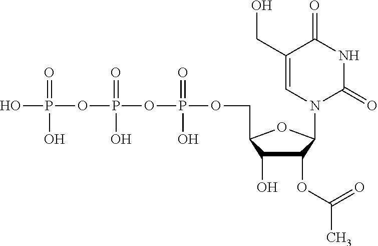

2′-O-acetyl-1-methylpseudouridine

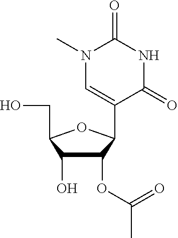

2′-O-acetyl-1-methylpseudouridine
monophosphate

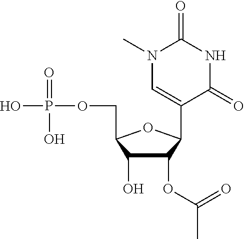

2′-O-acetyl-1-methylpseudouridine
diphosphate

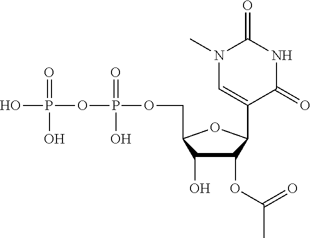

2′-O-acetyl-1-methylpseudouridine
triphosphate

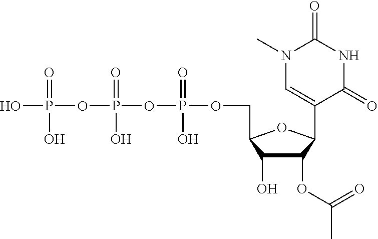

2′-O-acetyluridine:

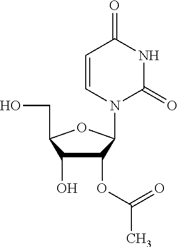

2′-O-acetyluridine monophosphate:

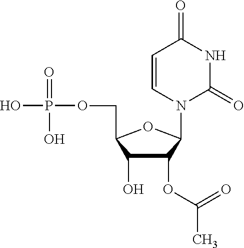

2′-O-acetyluridine diphosphate:

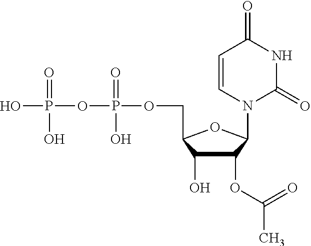

2′-O-acetyluridine triphosphate:

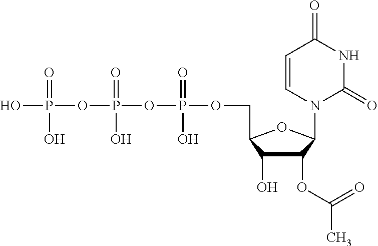

2′-O-acetylcytidine:

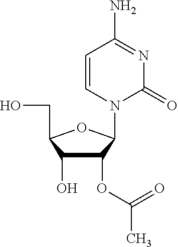

2′-O-acetylcytidine
monophosphate:

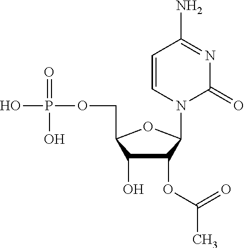

2′-O-acetylcytidine diphosphate:

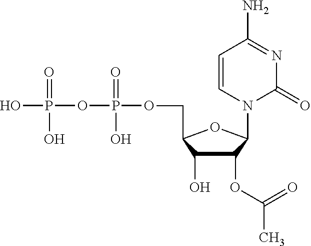

2′-O-acetylcytidine triphosphate:

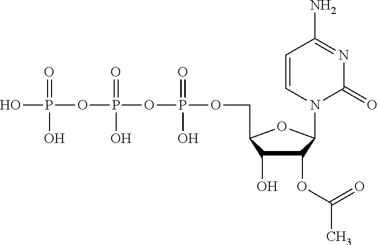

2′-O-acetylguanosine:

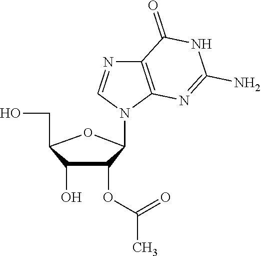

2′-O-acetylguanosine
monophosphate:

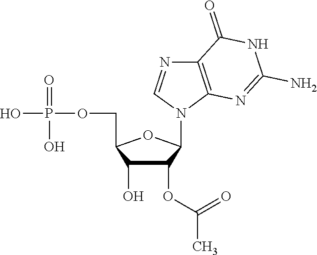

2′-O-acetylguanosine
diphosphate:

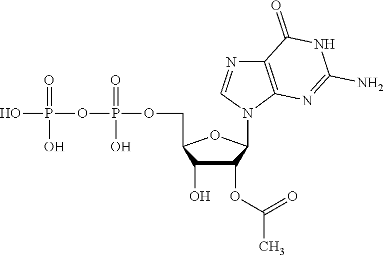

2′-O-acetylguanosine triphosphate:

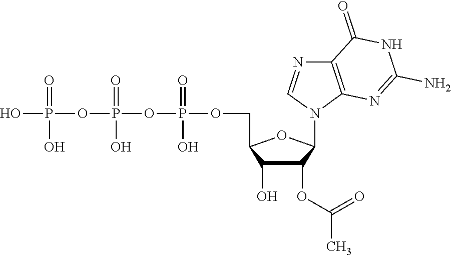

2′-O-acetyladenosine:

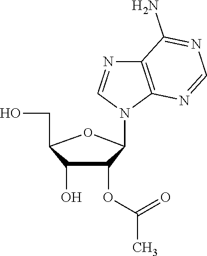

2′-O-acetyladenosine
monophosphate:

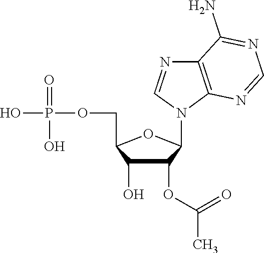

2′-O-acetyladenosine diphosphate:

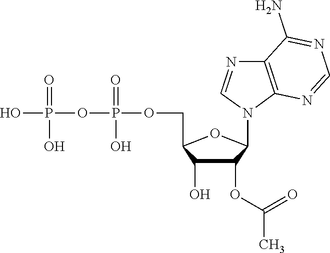

2′-O-acetyladenoosine triphosphate:

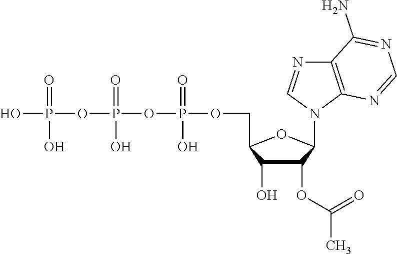

TABLE 3D
Exemplary modified ribonucleotides (when incorporated
into a polyribonucleotide)
2′-O-acetyl N4-acetylcytidine:

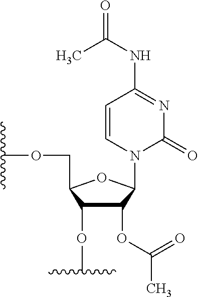

2′-O-acetyl N4-acetylcytidine
monophosphate:

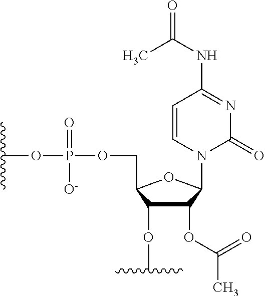

2′-O-acetyl N4-acetylcytidine
diphosphate:

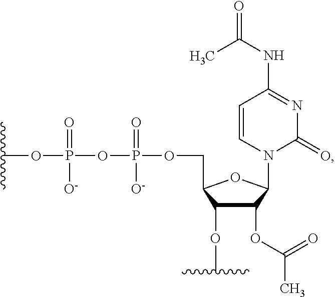

2′-O-acetyl N4-acetylcytidine
triphosphate:

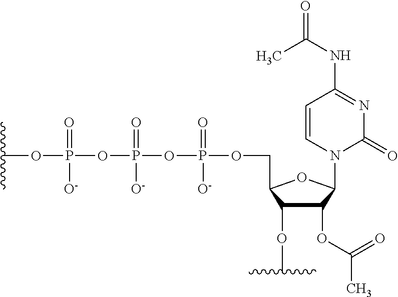

2′-O-acetyl 5-
hydroxymethyluridine:

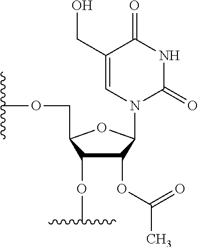

2′-O-acetyl 5-
hydroxymethyluridine
monophosphate:

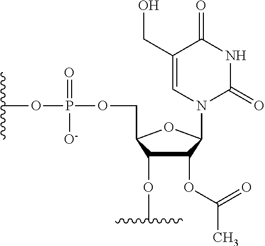

2′-O-acetyl 5-
hydroxymethyluridine
diphosphate:

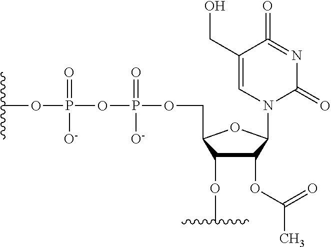

2′-O-acetyl 5-
hydroxymethyluridine
triphosphate:

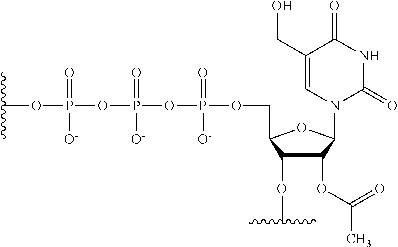

2′-O-acetyluridine:

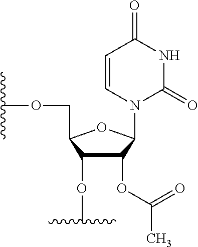

2′-O-acetyluridine
monophosphate:

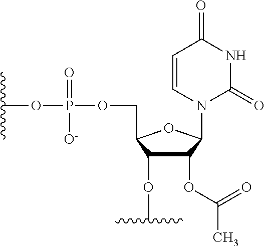

2′-O-acetyluridine diphosphate:

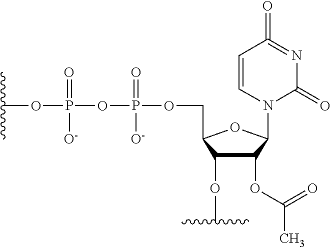

2′-O-acetyluridine triphosphate:

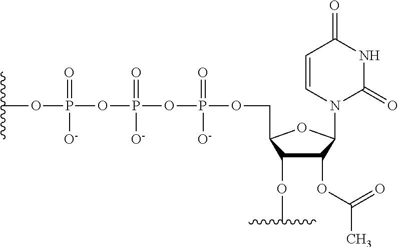

2′-O-acetylcytidine:

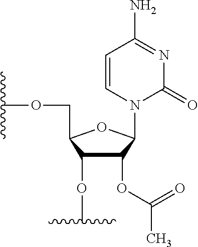

2′-O-acetylcytidine
monophosphate:

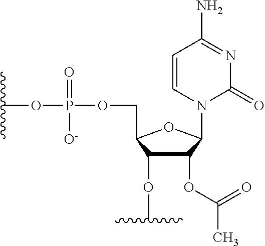

2′-O-acetylcytidine diphosphate:

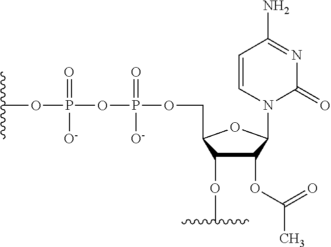

2′-O-acetylcytidine triphosphate:

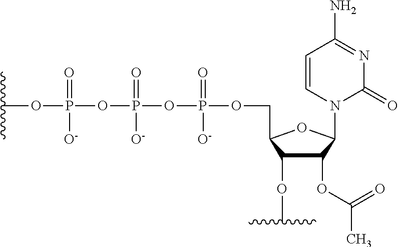

2′-O-acetylguanosine:

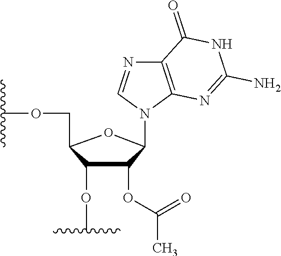

2′-O-acetylguanosine
monophosphate:

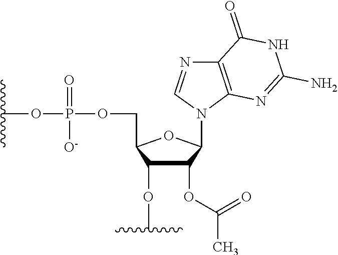

2′-O-acetylguanosine
diphosphate:

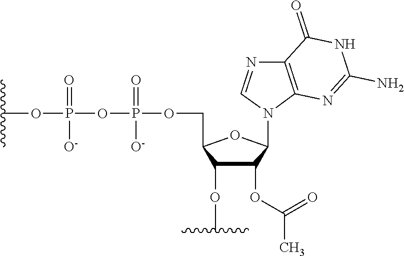

2′-O-acetylguanosine
triphosphate:

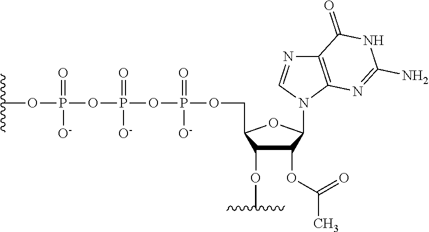

2′-O-acetyladenosine:

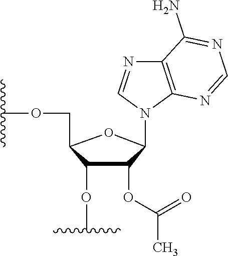

2′-O-acetyladenosine
monophosphate:

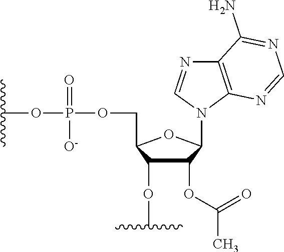

2′-O-acetyladenosine
diphosphate:

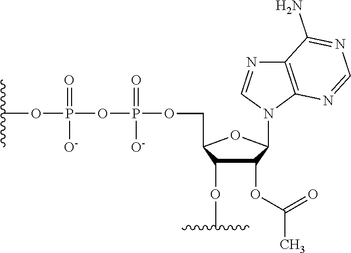

2′-O-acetyladenoosine
triphosphate:

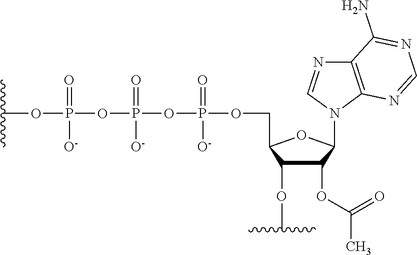

2′-O-acetyl-1-
methylpseudouridine

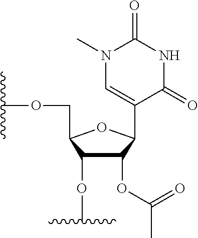

2′-O-acetyl-1-
methylpseudouridine
monophosphate

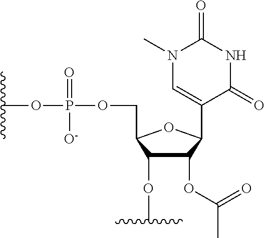

2′-O-acetyl-1-
methylpseudouridine
diphosphate

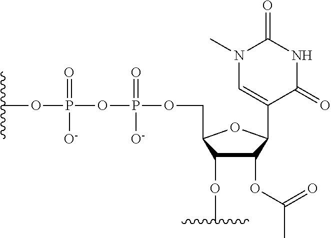

2′-O-acetyl-1-
methylpseudouridine
triphosphate

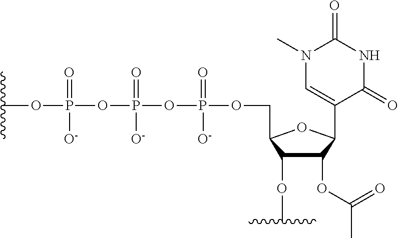

NOTE: [symbol]
indicates the position of attachment to an adjacent ribonucleotide.

In some embodiments, at least 5% of ribose moieties of a polyribonucleotide are 2′-O-acetylated.

In some embodiments, about 5% to about 99% of the ribose moieties of a polyribonucleotide are 2′-O-acetylated.

In some embodiments, a polyribonucleotide disclosed herein comprises a cap structure and the cap structure does not comprise a 2′-O-acetylated ribose.

In some embodiments, a polyribonucleotide disclosed herein comprises a cap structure and the cap structure comprises a 2′-O-acetylated ribose.

In some embodiments, a polyribonucleotide disclosed herein further comprises one or more ribonucleotides that does not comprise a 2′-0 acetylated ribose.

### RNA Encoding Fusion Polypeptides

Among other things, provided herein are polypeptides and polynucleotides which can be used to stimulate an immune response against an antigen and/or to enhance immunogenicity of an antigen.

In some embodiments, a polypeptide disclosed herein is encoded by a polynucleotide comprising an RNA. In some embodiments, a polynucleotide comprises a messenger RNA.

### Cap Structures

In some embodiments of any of the polyribonucleotides disclosed herein, a polyribonucleotide comprises a cap structure.

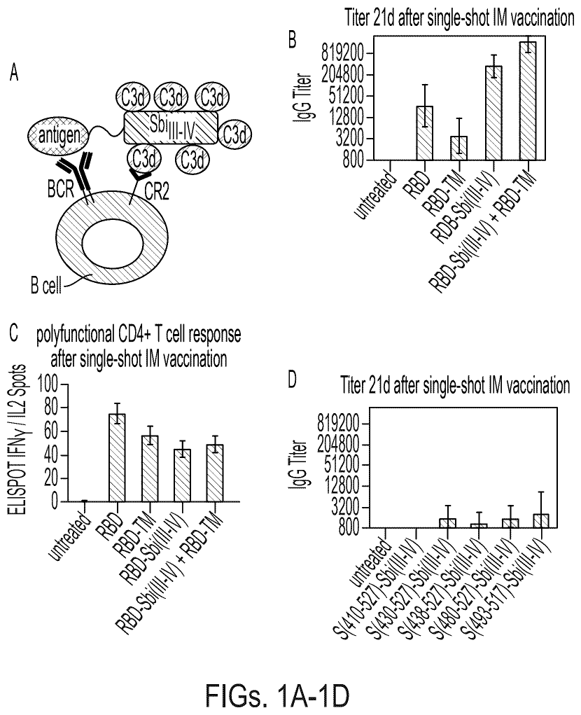

Prior versions of RNA capping have been described, e.g., in Decroly E et al. (2012) Nature Reviews 10: 51-65; and in Ramanathan A. et al., (2016) Nucleic Acids Res, 44(16): 7511-7526, the entire contents of each of which is hereby incorporated by reference. 5′ caps include a Cap-0 (also referred herein as “Cap0”, a Cap-1 (also referred herein as “Cap1”, or Cap-2 (also referred herein as “Cap2”). See, e.g., FIG. 1 of Ramanathan A et al., and FIG. 1 of Decroly E et al.

The term “5′-cap” as used herein refers to a structure found on the 5′-end of an RNA, e.g., mRNA, and generally includes a guanosine nucleotide connected to an RNA, e.g., mRNA, via a 5′- to 5-triphosphate linkage (also referred to as Gppp or G(5′)ppp(5′)).

In some embodiments of a polyribonucleotide disclosed herein, the 5′ end of the polyribonucleotide comprises a compound of formula I:

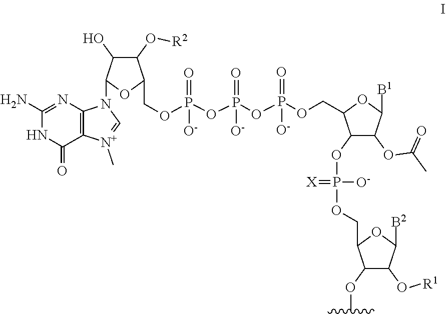

or a salt thereof, wherein:
[symbol] indicates the position at which the compound is attached to the polyribonucleotide;
each of B¹ and B² is independently selected from a natural base and a modified base;
R¹ is selected from hydrogen, C₁₋₆ alkyl, and —C(═O)CH₃;
R² is hydrogen or —CH₃; and
X is O or S.

As defined generally above for formula I, each of B¹ and B² is independently selected from a natural base and a modified base. In some embodiments, B¹ is a natural base. In some embodiments, B¹ is adenine. In some embodiments, B¹ is cytosine. In some embodiments, B¹ is guanine. In some embodiments, B¹ is uracil.

In some embodiments, B¹ is a modified base. In some embodiments, B¹ is selected from N1-methylpseudouracil, a pyridin-4-one, 5-aza-uracil, 6-aza-uracil, 2-thio-5-aza-uracil, 2-thio-uracil, 5-methyl cytosine, 5-aza-cytosine, 6-aza-cytosine, pseudoisocytosine, 3-methyl-cytosine, 5-formyl-cytosine, N4-methyl-cytosine, a 2-amino-purine, a 2,6-diaminopurine, a 2-amino-6-halo-purine, a 6-halo-purine, hypoxanthine, 1-methyl-hypoxanthine, 4,6-dimethyl-3,3a,4,9a-tetrahydro-9H-imidazo[1,2-a]purin-9-one, 4,6,7-trimethyl-3,3a,4,9a-tetrahydro-9H-imidazo[1,2-a]purin-9-one, N4-acetylcytosine, and 5-hydroxymethyluracil.

In some embodiments, B² is a natural base. In some embodiments, B² is adenine. In some embodiments, B² is cytosine. In some embodiments, B² is guanine. In some embodiments, B² is uracil.

In some embodiments, B² is a modified base. In some embodiments, B² is selected from N1-methylpseudouracil, a pyridin-4-one, 5-aza-uracil, 6-aza-uracil, 2-thio-5-aza-uracil, 2-thio-uracil, 5-methyl cytosine, 5-aza-cytosine, 6-aza-cytosine, pseudoisocytosine, 3-methyl-cytosine, 5-formyl-cytosine, N4-methyl-cytosine, a 2-amino-purine, a 2,6-diaminopurine, a 2-amino-6-halo-purine, a 6-halo-purine, hypoxanthine, 1-methyl-hypoxanthine, 4,6-dimethyl-3,3a,4,9a-tetrahydro-9H-imidazo[1,2-a]purin-9-one, 4,6,7-trimethyl-3,3a,4,9a-tetrahydro-9H-imidazo[1,2-a]purin-9-one, N4-acetylcytosine, and 5-hydroxymethyluracil.

As defined generally above for formula I, R¹ is selected from hydrogen, C₁₋₆ alkyl, and —C(═O)CH₃. In some embodiments, R¹ is hydrogen. In some embodiments, R¹ is C₁₋₆ alkyl. In some embodiments, R¹ is —CH₃. In some embodiments, R¹ is

—C(═O)CH₃.

As defined generally above for formula I, R² is hydrogen or —CH₃. In some embodiments, R² is hydrogen. In some embodiments, R² is —CH₃.

As defined generally above for formula I, X is O or S. In some embodiments, X is O. In some embodiments, X is S.

In some embodiments, the present disclosure provides a compound of any of formulae I-a, I-b, I-c, I-d, I-e, and I-f:

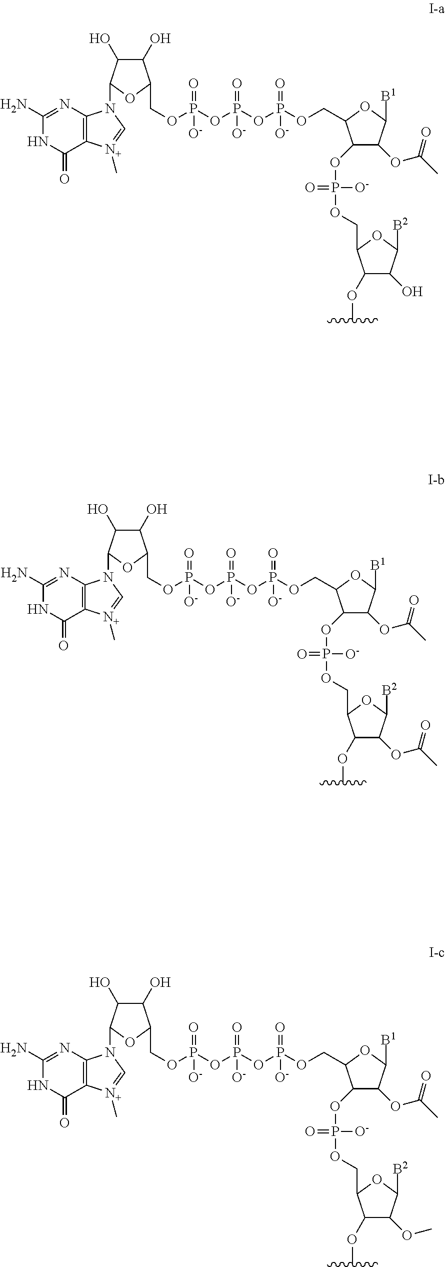

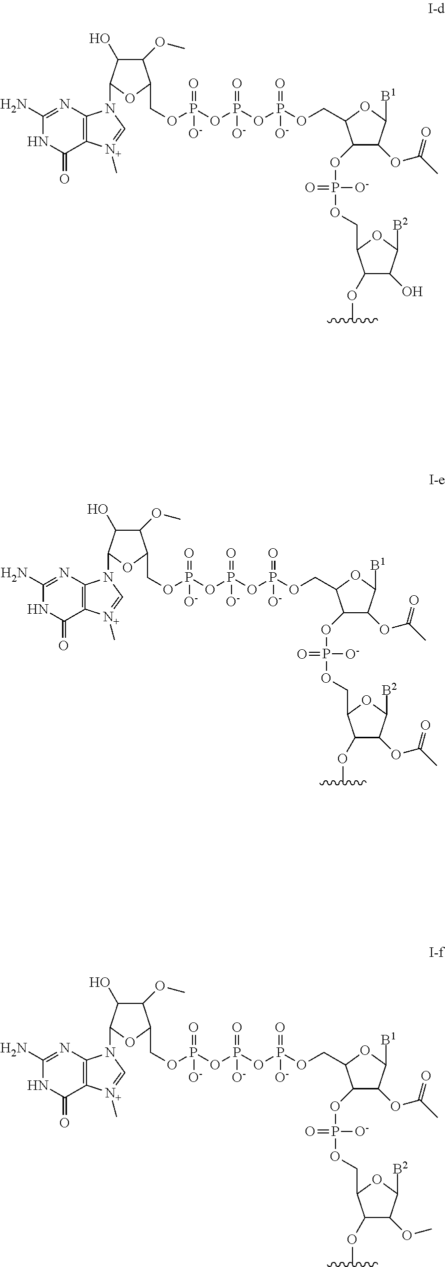

or a salt thereof, wherein each of B¹ and B² is as defined above and described herein.

In some embodiments, a compound of formula I is selected from

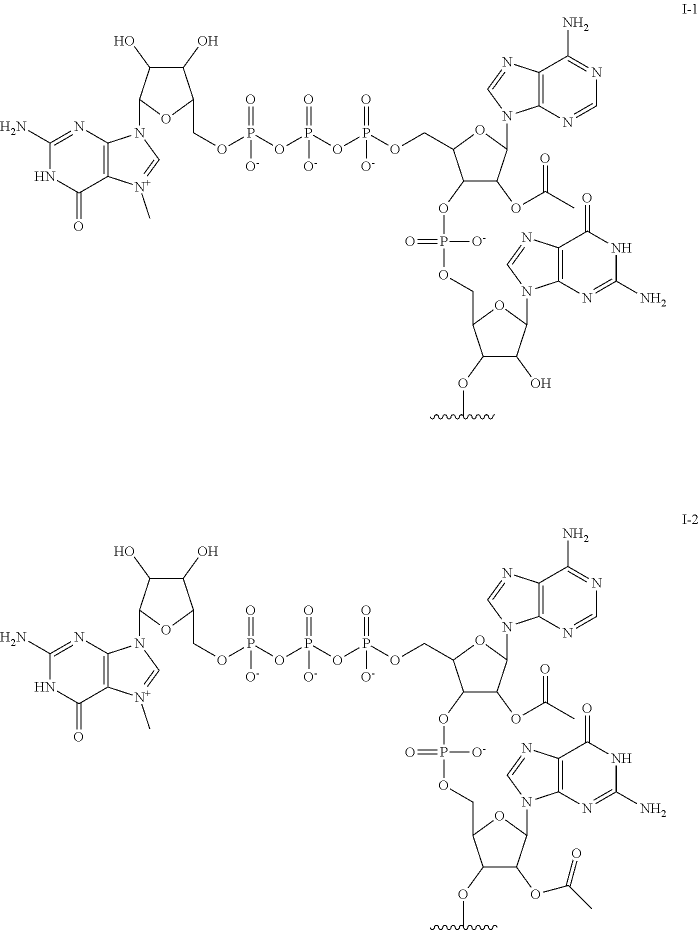 [SMILES: *OC1C(COP(=O)([O-])OC2C(COP(=O)([O-])OP(=O)([O-])OP(=O)([O-])OCC3OC(n4c[n+](C)c5c(=O)[nH]c(N)nc54)C(O)C3O)OC(n3cnc4c(N)ncnc43)C2OC(C)=O)OC(n2cnc3c(=O)[nH]c(N)nc32)C1O ; *OC1C(COP(=O)([O-])OC2C(COP(=O)([O-])OP(=O)([O-])OP(=O)([O-])OCC3OC(n4c[n+](C)c5c(=O)[nH]c(N)nc54)C(O)C3O)OC(n3cnc4c(N)ncnc43)C2OC(C)=O)OC(n2cnc3c(=O)[nH]c(N)nc32)C1OC(C)=O]

or a salt thereof.

In some embodiments, a polyribonucleotide disclosed herein comprises at the 5′ end a ^{m7}GpppN₁pN₂—OH cap with the following structure:

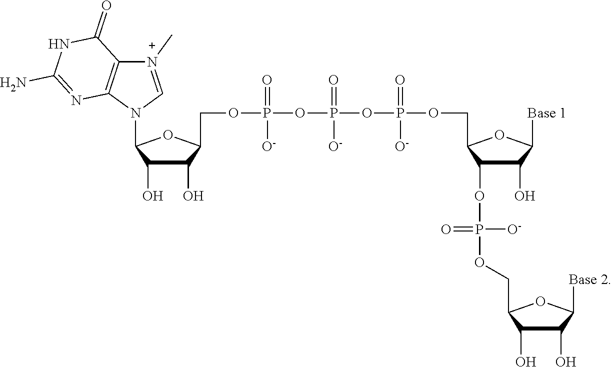

In some embodiments, a polyribonucleotide disclosed herein comprises at the 5′ end a compound with the following structure:

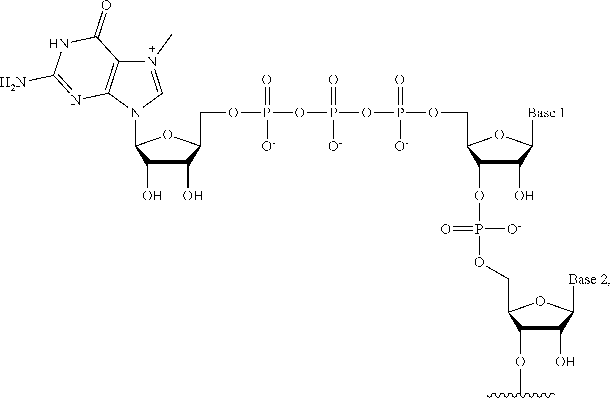

wherein [symbol] indicates the position at which the compound is attached to the polyribonucleotide.

In some embodiments, a polyribonucleotide disclosed herein comprises a ^{m7}GpppN₁^{(2′-OMe)}pN₂—OH cap with the following structure:

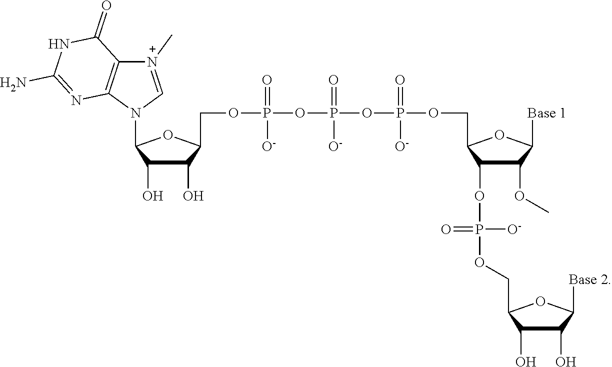

In some embodiments, a polyribonucleotide disclosed herein comprises at the 5′ end a compound with the following structure:

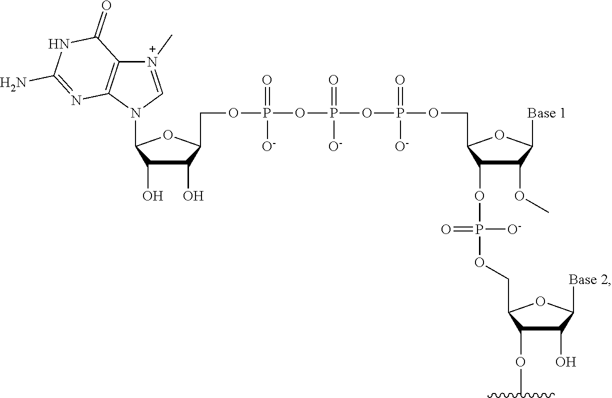

wherein [symbol] indicates the position at which the compound is attached to the polyribonucleotide.

In some embodiments, a polyribonucleotide disclosed herein comprises a ^{m7}GpppN₁^{(2′-OMe)}pN₂^{(2′-OMe)}-OH cap with the following structure:

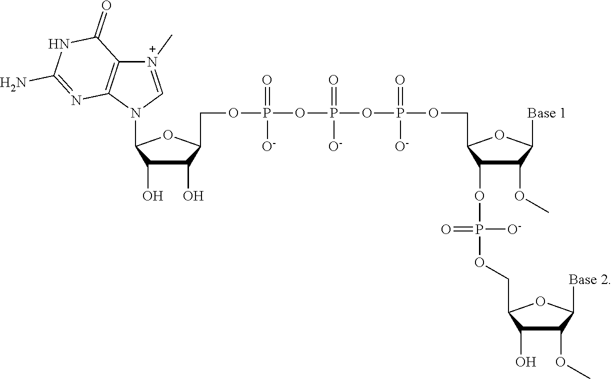

In some embodiments, a polyribonucleotide disclosed herein comprises at the 5′ end a compound with the following structure:

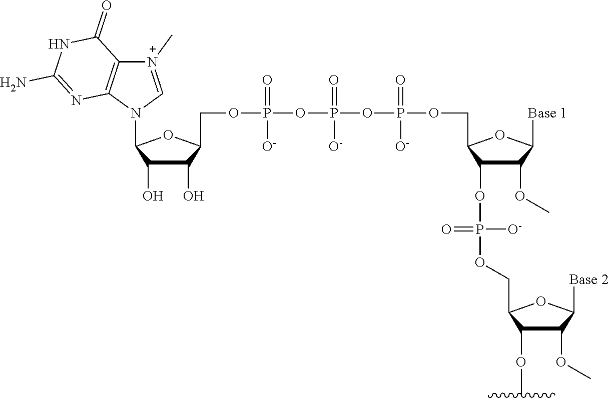

wherein [symbol] indicates the position at which the compound is attached to the polyribonucleotide.

In some embodiments, for any of the cap structures disclosed herein, each of B¹ and B² is independently selected from a natural base and a modified base. In some embodiments, B¹ is a natural base. In some embodiments, B¹ is adenine. In some embodiments, B¹ is cytosine. In some embodiments, B¹ is guanine. In some embodiments, B¹ is uracil.

In some embodiments, B¹ is a modified base. In some embodiments, B¹ is selected from N1-methylpseudouracil, a pyridin-4-one, 5-aza-uracil, 6-aza-uracil, 2-thio-5-aza-uracil, 2-thio-uracil, 5-methyl cytosine, 5-aza-cytosine, 6-aza-cytosine, pseudoisocytosine, 3-methyl-cytosine, 5-formyl-cytosine, N4-methyl-cytosine, a 2-amino-purine, a 2,6-diaminopurine, a 2-amino-6-halo-purine, a 6-halo-purine, hypoxanthine, 1-methyl-hypoxanthine, 4,6-dimethyl-3,3a,4,9a-tetrahydro-9H-imidazo[1,2-a]purin-9-one, 4,6,7-trimethyl-3,3a,4,9a-tetrahydro-9H-imidazo[1,2-a]purin-9-one, N4-acetylcytosine, and 5-hydroxymethyluracil.

In some embodiments, B² is a natural base. In some embodiments, B² is adenine. In some embodiments, B² is cytosine. In some embodiments, B² is guanine. In some embodiments, B² is uracil.

In some embodiments, B² is a modified base. In some embodiments, B² is selected from N1-methylpseudouracil, a pyridin-4-one, 5-aza-uracil, 6-aza-uracil, 2-thio-5-aza-uracil, 2-thio-uracil, 5-methyl cytosine, 5-aza-cytosine, 6-aza-cytosine, pseudoisocytosine, 3-methyl-cytosine, 5-formyl-cytosine, N4-methyl-cytosine, a 2-amino-purine, a 2,6-diaminopurine, a 2-amino-6-halo-purine, a 6-halo-purine, hypoxanthine, 1-methyl-hypoxanthine, 4,6-dimethyl-3,3a,4,9a-tetrahydro-9H-imidazo[1,2-a]purin-9-one, 4,6,7-trimethyl-3,3a,4,9a-tetrahydro-9H-imidazo[1,2-a]purin-9-one, N4-acetylcytosine, and 5-hydroxymethyluracil.

### Formulations

In some embodiments, a polynucleotide comprising an RNA is formulated in a lipid nanoparticle (LNP) formulation.

In some embodiments, the disclosure provides an LNP formulation comprising a polynucleotide comprising an RNA for use in an immunogenic composition.

In some embodiments, an LNP formulation comprising a polynucleotide comprising an RNA is administered to a subject to enhance and/or modulate an immune response. In some embodiments, the immune response is elicited by an antigen comprised in the polynucleotide.

In some embodiments, an LNP formulation comprising a polynucleotide comprising an RNA is characterized in that when administered to a subject, it reduces immunogenicity to an antigen, e.g., an antigen comprised in a polynucleotide or an antigen comprised in a polypeptide. In some embodiments, the immunogenicity that is reduced is the immunogenicity in response to the RNA molecules themselves caused by administration of RNA therapeutics and not immunogenicity caused by, e.g., polypeptides encoded by the RNA molecules, which may be desirable as a result of, e.g., an RNA vaccine.

In some embodiments, an LNP formulation comprising a polynucleotide comprising an RNA is characterized in that when administered to a subject it focuses the immune response on a conserved and/or functional region of an epitope of a target protein antigen.

In some embodiments, an LNP formulation comprising a polynucleotide comprising an RNA is characterized in that when administered to a subject it improves the breadth and/or efficacy of an immune response in the subject to a target protein antigen.

In some embodiments, an LNP formulation comprising a polynucleotide comprising an RNA is characterized in that when administered to a subject it results in a humoral response, (e.g., a broadly neutralizing humoral response), and/or a T cell mediated response.

In some embodiments, a polypeptide disclosed herein comprises a fusion polypeptide as described herein. In some embodiments, a polynucleotide disclosed herein comprises an RNA. In some embodiments, a polynucleotide comprises a messenger RNA.

In some embodiments, a polynucleotide comprising an RNA is formulated in a lipid nanoparticle (LNP) formulation.

In some embodiments, the disclosure provides an LNP formulation comprising a polynucleotide comprising an RNA for use in an immunogenic composition.

In some embodiments, an LNP formulation comprising a polynucleotide comprising an RNA is administered to a subject to enhance and/or modulate an immune response. In some embodiments, the immune response is elicited by an antigen, e.g., an antigen fragment, an antigen variant, or an antigen fragment variant, encoded by a polynucleotide. In some embodiments, the immune response is enhanced by an adjuvant, e.g., C3d binding polypeptide, encoded by a polynucleotide.

In some embodiments, an LNP formulation comprising a polynucleotide comprising an RNA is characterized in that when administered to a subject it focuses the immune response on a conserved and/or functional region of an encoded epitope of a target protein antigen.

In some embodiments, an LNP formulation comprising a polynucleotide comprising an RNA is characterized in that when administered to a subject it improves the breadth and/or efficacy of an immune response in the subject to a target protein antigen.

In some embodiments, an LNP formulation comprising a polynucleotide comprising an RNA is characterized in that when administered to a subject it results in a humoral response, (e.g., a broadly neutralizing humoral response), and/or a T cell mediated response.

### Compositions

Among other things, the present disclosure provides compositions. Compositions disclosed herein, e.g., compositions comprising a polypeptide or a polynucleotide disclosed herein, can focus the immune response on conserved and/or functional regions of an antigen. In some embodiments, this immunofocusing can improve the breadth and/or efficacy of an immune response against an antigen.

In some embodiments, a composition disclosed herein is characterized in that when administered to a subject, it increases immunogenicity to an antigen, e.g., an antigen encoded by a polynucleotide or an antigen comprised in a fusion polypeptide.

In some embodiments, a composition disclosed herein is characterized in that when administered to a subject it focuses the immune response on a conserved and/or functional region of an epitope of a target protein antigen.

In some embodiments, a composition disclosed herein is characterized in that when administered to a subject it improves the breadth and/or efficacy of an immune response in the subject to a target protein antigen.

In some embodiments, a composition disclosed herein is characterized in that when administered to a subject it results in a humoral response, (e.g., a broadly neutralizing humoral response). In some embodiments, a humoral response is elicited in a subject that does not have functional T cells or in a subject that does not have T cells.

In some embodiments, a composition disclosed herein is characterized in that when administered to a subject it results in a T cell mediated response.

In some embodiments, a composition comprises a polypeptide as described herein, e.g., a fusion polypeptide as described herein. In some embodiments, a composition comprises a polynucleotide.

In some embodiments, a composition is or comprises a pharmaceutical composition, e.g., as described herein.

In some embodiments, a composition is or comprises an expression vector comprising a polynucleotide disclosed herein.

In some embodiments, a composition is or comprises an immunogenic composition, e.g., as described herein.

In some embodiments, a composition described herein can be delivered, e.g., administered, to a subject in combination with one or more other compositions.

### Immunogenic Compositions

Disclosed herein are immunogenic compositions comprising (1) an antigen (e.g., an antigen fragment, an antigen variant or an antigen fragment variant); and/or (2) an adjuvant comprising a complement C3d-binding region. In some embodiments, an immunogenic composition disclosed herein can enhance the titers of the resulting antibody response. In some embodiments, an immunogenic composition disclosed herein can result in a measurable T cell response.

In some embodiments, an immunogenic composition comprises an immunoglobulin-binding protein (Sbi) of Staphylococcus aureus for use as an adjuvant. In some embodiments, an immunogenic composition comprising Sbi for use as an adjuvant comprises an Sbi polypeptide or a fragment thereof. In some embodiments, an immunogenic composition comprising Sbi for use as an adjuvant comprises a polynucleotide encoding an Sbi polypeptide or a fragment thereof.

In some embodiments, an immunogenic composition comprises an antigen, e.g., an antigen fragment, an antigen variant, or an antigen fragment variant, comprising an epitope of a target protein antigen, and an adjuvant comprising a complement C3d-binding polypeptide.

In some embodiments, an immunogenic composition comprises a fusion polypeptide comprising an antigen, e.g., an antigen fragment, an antigen variant, or an antigen fragment variant, comprising an epitope of a target protein antigen joined (e.g., directly or indirectly) to a complement C3d-binding polypeptide. In some embodiments, a C3d-binding polypeptide comprises an immunoglobulin-binding protein (Sbi) of Staphylococcus aureus. In some embodiments, a complement C3d-binding polypeptide is or comprises one or both of domain III and domain IV of Sbi of Staphylococcus aureus, or a functional fragment or a variant thereof of any of the foregoing.

In some embodiments, an immunogenic composition comprises a polynucleotide encoding a fusion polypeptide comprising an antigen, e.g., an antigen fragment, an antigen variant or an antigen fragment variant, comprising an epitope of a target protein antigen joined (e.g., directly or indirectly) to a complement C3d-binding polypeptide. In some embodiments, a polynucleotide encodes a C3d binding polypeptide comprising an immunoglobulin-binding protein (Sbi) of Staphylococcus aureus. In some embodiments, a complement C3d-binding polypeptide is or comprises one or both of domain III and domain IV of the Sbi of Staphylococcus aureus, or a functional fragment or a variant thereof of any of the foregoing.

In some embodiments, an immunogenic composition comprises an expression vector comprising a polynucleotide disclosed herein.

### Pharmaceutical Compositions

Pharmaceutical compositions of the present disclosure may comprise a polypeptide disclosed herein (e.g., a fusion polypeptide), a polynucleotide disclosed herein, or an expression vector comprising a polynucleotide. In some embodiments, a pharmaceutical composition may comprise a pharmaceutically acceptable excipient, a diluent, or a combination thereof. In some embodiments, a pharmaceutical composition may comprise buffers such as neutral buffered saline, phosphate buffered saline and the like; carbohydrates such as glucose, mannose, sucrose, or dextrans; mannitol; proteins; polypeptides or amino acids such as glycine; antioxidants; chelating agents such as EDTA or glutathione; and preservatives.

In some embodiments, a pharmaceutical composition is formulated for administration according to any of the routes of administration disclosed herein. In some embodiments, a pharmaceutical composition is formulated for intramuscular administration, intradermal administration, intravenous administration, or subcutaneous administration.

In some embodiments, a pharmaceutical composition described herein comprises one or more compositions, e.g., a first composition and an additional composition [e.g., a second composition]. In some embodiments, a first composition comprises a first fusion polynucleotide encoding a first fusion polypeptide, or a first fusion polypeptide. In some embodiments, a second composition comprises a second fusion polynucleotide encoding a second fusion polypeptide, or a second fusion polypeptide. In some embodiments a first fusion polynucleotide encodes one or more antigens. In some embodiments a second fusion polynucleotide encodes one or more antigens. In some embodiments a first fusion polynucleotide encodes one or more antigens that are different than the one or more antigens encoded by the second fusion polynucleotide. In some embodiments a first fusion polynucleotide encodes one or more antigens that are the same as the one or more antigens encoded by the second fusion polynucleotide.

In some embodiments, a second composition is administered prior to administration of a first composition. In some embodiments, a second composition is administered after administration of a first composition. In some embodiments, a second composition is administered concurrently with a first composition.

In some embodiments, a pharmaceutical composition described herein can be administered with an additional therapeutic agent, e.g., a standard of care for one or more disorders disclosed herein.

In some embodiments, an additional therapeutic agent is an agent useful in treating, preventing, and/or reducing (e.g., a frequency and/or severity) of one or more symptoms associated with one or more disorders disclosed herein. In some embodiments, an agent useful in treating, preventing, and/or reducing (e.g., a frequency and/or severity) of one or more symptoms associated with one or more disorders disclosed herein comprises: a biologic agent, a small molecule agent, and/or nucleic acid agent. In some embodiments, a biologic agent comprises a polypeptide agent or a cell-based therapy. In some embodiments, a biologic agent comprises a B cell targeting therapy. In some embodiments, a B cell targeting therapy comprises an antibody, e.g., rituximab, belimumab, an IgG depleting antibody.

In some embodiments, a small molecule agent comprises a T cell inhibitor (e.g., calcineurin inhibitors [e.g., voclosporin]), an interferon receptor inhibitor (e.g., anifrolumab), a JAK inhibitor, a TYK2 inhibitor, an immunosuppressive agent (e.g., mycophenolate mofetil, methotrexate), or any combination thereof.

In some embodiments, an agent useful in treating, preventing, and/or reducing (e.g., a frequency and/or severity) of one or more symptoms associated with one or more disorders disclosed herein comprises: an immunosuppressive agent, a nonsteroidal anti-inflammatory agent, an anti-malarial agent, a corticosteroid agent, or any combination thereof. In some embodiments, a corticosteroid comprises prednisone, or methyl-prednisone.. In some embodiments, an immunosuppressive agent comprises mycophenolate mofetil, methotrexate, or any combination thereof.

### Methods of Using Compositions Disclosed Herein

The disclosure provides, among other things, methods for using a fusion polypeptide described herein, a polynucleotide described herein, or a composition comprising the same to stimulate an immune response against an antigen (e.g., as a vaccine), or to enhance immunogenicity of an antigen, e.g., in an immunosuppressed subject.

In some embodiments, administration of a fusion polypeptide described herein, a polynucleotide described herein, or a composition comprising the same to a subject, e.g., an immunosuppressed subject, induces an immune response against an antigen, e.g., antigen fragment, antigen variant, or antigen fragment variant, in the subject.

In some embodiments, administration of a fusion polypeptide described herein, a polynucleotide described herein, or a composition comprising the same to a subject, e.g., an immunosuppressed subject, stimulates B cells in a subject. In some embodiments, stimulation of B cells occurs in the absence of detectable T cell stimulation. In some embodiments, stimulation of B cells results in a humoral response.

Use of compositions disclosed herein, e.g., compositions comprising a fusion polypeptide or a polynucleotide disclosed herein, can focus the immune response on conserved and/or functional regions of an antigen. In some embodiments, this immunofocusing can improve the breadth and/or efficacy of an immune response against the antigen.

In some embodiments, a method comprising administering a composition disclosed herein results in reduced immunogenicity to an antigen, e.g., an antigen encoded by a polynucleotide or an antigen comprised in a fusion polypeptide. In some embodiments, the immunogenicity that is reduced is the immunogenicity in response to the RNA molecules themselves caused by administration of RNA therapeutics and not immunogenicity caused by, e.g., polypeptides encoded by the RNA molecules, which may be desirable as a result of, e.g., an RNA vaccine.

In some embodiments, a method comprising administering a composition disclosed herein results in focusing of an immune response on a conserved and/or functional region of an epitope of a target protein antigen.

In some embodiments, a method comprising administering a composition disclosed herein results in improved breadth and/or efficacy of an immune response in a subject to a target protein antigen.

In some embodiments, a method comprising administering a composition disclosed herein results in a humoral response, (e.g., a broadly neutralizing humoral response). In some embodiments, a humoral response is stimulated in the absence of T cells. In some embodiments, a humoral response is stimulated in the presence of defective or non-functional T cells.

This disclosure provides a method comprising administering to a subject in need thereof at least one dose of a pharmaceutical composition comprising a fusion polypeptide disclosed herein, a polynucleotide disclosed herein, or an expression vector comprising a polynucleotide disclosed herein. In some embodiments, the at least one dose is administered in an effective amount to induce an immune response against an antigen, e.g., an antigen fragment, antigen variant, or antigen fragment variant, in the subject.

Disclosed herein is a method comprising administering to a subject: a first dose of a pharmaceutical composition disclosed herein; and a second dose of a pharmaceutical composition disclosed herein. In some embodiments, a pharmaceutical composition comprises a fusion polypeptide disclosed herein, a polynucleotide disclosed herein, or an expression vector comprising a polynucleotide disclosed herein.

In some embodiments, a first dose and a second dose are administered by at least 3 days, at least 4 days, at least 5 days, at least 6 days, at least 7 days, at least 2 weeks, at least 3 weeks, or at least 4 weeks apart.

In some embodiments, a first dose and a second dose are in the same amount. In some embodiments, a first dose and a second dose are in different amounts.

This disclosure provides, a method for enhancing the immunogenicity of an antigen, comprising administering to a subject in need thereof, a fusion polypeptide disclosed herein, or a polynucleotide disclosed herein, or a pharmaceutical composition disclosed herein.

This disclosure also provides a method for stimulating an immune response against an antigen, comprising administering to a subject in need thereof, a fusion polypeptide disclosed herein, or a polynucleotide disclosed herein, or a pharmaceutical composition disclosed herein.

In some embodiments of any of the methods or uses disclosed herein, a single dose of a fusion polypeptide, polynucleotide or pharmaceutical composition is administered. In some embodiments of any of the methods or uses disclosed herein, a plurality of doses of a fusion polypeptide, polynucleotide or pharmaceutical composition is administered.

In some embodiments of any of the methods or uses disclosed herein, a fusion polypeptide, polynucleotide or pharmaceutical composition is administered at a dose of about 0.5 micrograms (mg) to about 1500 mg, about 0.5 mg to about 1000 mg, about 0.5 mg to about 900 mg, about 0.5 mg to about 800 mg, about 0.5 mg to about 700 mg, about 0.5 mg to about 600 mg, about 0.5 mg to about 500 mg, about 0.5 mg to about 400 mg, about 0.5 mg to about 300 mg, about 0.5 mg to about 250 mg, about 0.5 mg to about 200 mg, about 0.5 mg to about 150 mg, about 0.5 mg to about 100 mg, about 0.5 mg to about 50 mg, about 0.5 mg to about 20 mg, about 0.5 mg to about 10 mg, about 0.5 mg to about 5 mg, about 0.5 mg to about 1 mg, about 1 mg to about 1500 mg, about 5 mg to about 1500 mg, about 10 mg to about 1500 mg, about 20 mg to about 1500 mg, about 50 mg to about 1500 mg, about 100 mg to about 1500 mg, about 150 mg to about 1500 mg, about 200 mg to about 1500 mg, about 250 mg to about 1500 mg, about 300 mg to about 1500 mg, about 400 mg to about 1500 mg, about 500 mg to about 1500 mg, or about 1000 mg to about 1500 mg.

In some embodiments, a fusion polypeptide, polynucleotide or pharmaceutical composition is administered at a dose of about 0.5 mg, about 1 mg, about 10 mg, about 20 mg, about 50 mg, about 100 mg, about 150 mg, about 200 mg, about 250 mg, about 300 mg, about 400 mg, about 500 mg, about 1000 mg, or about 1500 mg.

In some embodiments, a fusion polypeptide, polynucleotide or pharmaceutical composition is administered at a dose of at least 0.5 mg, at least 1 mg, at least 10 mg, at least 20 mg, at least 50 mg, at least 100 mg, at least 150 mg, at least 200 mg, at least 250 mg, at least 300 mg, at least 400 mg, at least 500 mg, at least 1000 mg, or at least 1500 mg.

In some embodiments of any of the methods or uses disclosed herein, administration of the polynucleotide, fusion polypeptide or pharmaceutical composition results in a humoral response. In some embodiments, the humoral response is an antibody response.

In some embodiments of any of the methods or uses disclosed herein, administration of the polynucleotide, fusion polypeptide or pharmaceutical composition results in an increased an antibody response (e.g., a titer or a concentration of an antibody response). In some embodiments, the increase in titer is an increase of about 1.5-fold to about 500-fold, about 1.5 fold to about 400-fold, about 1.5 fold to about 300-fold, about 1.5 fold to about 200-fold, about 1.5 fold to about 100-fold, about 1.5 fold to about 50-fold, about 1.5 fold to about 20-fold, about 1.5 fold to about 10-fold, about 1.5 fold to about 5-fold, about 5-fold to about 500-fold, about 10-fold to about 500-fold, about 20-fold to about 500-fold, about 50-fold to about 500-fold, about 100-fold to about 500-fold, about 200-fold to about 500-fold, about 300-fold to about 500-fold, about 400-fold to about 500-fold.

In some embodiments, the increase in titer is an increase of about 1.5-fold, about 5-fold, about 10-fold, about 20-fold, about 50-fold, about 100-fold, about 200-fold, about 300-fold, about 400-fold, or about 500-fold.

In some embodiments, the increased titer of the antibody response is compared to administration of an otherwise similar polynucleotide that does not encode: (1) an Sbi domain III, or a fragment or variant thereof; (2) an Sbi domain IV, or a fragment or a variant thereof; or (3) both (1) and (2). In some embodiments, the increased titer of the antibody response is compared to administration of an otherwise similar fusion polypeptide that does not comprise: (1) an Sbi domain III, or a fragment or variant thereof; (2) an Sbi domain IV, or a fragment or a variant thereof; or (3) both (1) and (2). In some embodiments, the increased titer of the antibody response is compared to administration of an otherwise similar pharmaceutical composition that does not comprise a polynucleotide sequence encoding: (1) an Sbi domain III, or a fragment or variant thereof; (2) an Sbi domain IV, or a fragment or a variant thereof, or (3) both (1) and (2).

In some embodiments of any of the methods or uses disclosed herein, the composition is administered via any one of the following routes of administration: intramuscular, intravenous, subcutaneous, intrathecal, intradermal, ocular, intranasal, sublingual, or oral.

Other features of the invention will become apparent in the course of the following descriptions of exemplary embodiments, which are given for illustration of the invention and are not intended to be limiting thereof.

### ENUMERATED EMBODIMENTS

Embodiment 1. A polynucleotide encoding a fusion polypeptide, wherein the fusion polypeptide comprises:
(a) one or more antigens; and
(b) a complement C3d-binding polypeptide from an immunoglobulin-binding protein (Sbi) of Staphylococcus aureus,
wherein the polynucleotide comprises: a modified nucleobase, a modified deoxyribose, a modified ribose, a modified backbone, or any combination thereof.

Embodiment 2. The polynucleotide of embodiment 1, wherein the polynucleotide is or comprises RNA.

Embodiment 3. The polynucleotide of embodiment 1, wherein the polynucleotide is or comprises DNA.

Embodiment 4. The polynucleotide of any one of the preceding embodiments, wherein the complement C3d-binding polypeptide is or comprises one or both of domain III and domain IV of the Sbi of Staphylococcus aureus, or a functional fragment or a variant thereof.

Embodiment 5. The polynucleotide of any one of the preceding embodiments, wherein the one or more antigens comprises one or more antigen variants or one or more antigen fragment variants that comprises at least one modified amino acid compared to the target protein antigen.

Embodiment 6. The polynucleotide of any one of the preceding embodiments, wherein the one or more antigens comprises one or more antigen fragments, one or more antigen variants or one or more antigen fragment variants that is characterized in that, when expressed in vivo, the antigen fragment, antigen variant or antigen fragment variant binds to a Major Histocompatibility Complex (MHC) molecule.

Embodiment 7. The polynucleotide of any one of embodiments 1-5, wherein the one or more antigens comprises one or more antigen fragments, one or more antigen variants or one or more antigen fragment variants that is characterized in that, when expressed in vivo, the one or more antigen fragments, one or more antigen variants or one or more antigen fragments variants does not bind to an MHC molecule.

Embodiment 8. The polynucleotide of any one of the preceding embodiments, wherein the one or more antigens comprises one or more antigen fragments, one or more antigen variants or one or more antigen fragment variants which is or comprise:
(i) an infectious disease antigen; or
(ii) a cancer antigen.

Embodiment 9. The polynucleotide of embodiment 8, wherein the infectious disease antigen is or comprises a viral antigen, a bacterial antigen, a fungal antigen, or combinations thereof.

Embodiment 10: The polynucleotide of any one of the preceding embodiments, wherein the polypeptide encodes 2, 3, 4, 5, 6, 7, 8, 9, 10, 15 or 20 antigens.

Embodiment 11. The polynucleotide of any one of the preceding embodiments, wherein the one or more antigens are from the same pathogen.

Embodiment 12. The polynucleotide of any one of the preceding embodiments, wherein the one or more antigens are from different pathogens.

Embodiment 13: The polynucleotide of any one of the preceding embodiments, wherein the one or more antigens are from the same target protein antigen.

Embodiment 14: The polynucleotide of any one of embodiments 1-13, wherein the one or more antigens are from different target protein antigens.

Embodiment 15. The polynucleotide of any one of the preceding embodiments, wherein:
(i) (a) is disposed N-terminus of (b); or
(ii) (a) is disposed C-terminus of (b);
(iii) (a) and (b) are contiguous;
(iv) (a) and (b) are separated by a linker; or
(iv) a combination thereof.

Embodiment 16. The polynucleotide of embodiment 15, wherein:
(i) the linker is or comprises a peptidyl linker;
(ii) the linker is or comprises a peptidyl linker comprising at least 60% glycine and/or serine;
(iii) the linker is or comprises a Gly-Ser linker or a histidine linker.

Embodiment 17. The polynucleotide of embodiment 16, wherein the Gly-Ser linker is a Gly-Gly-Gly-Gly-Ser (Gly4-Ser) linker.

Embodiment 18. The polynucleotide of embodiment 17, wherein the linker comprises the amino acid sequence of SEQ ID NO: 6.

Embodiment 19. The polynucleotide of any one of the preceding embodiments, wherein the polynucleotide further comprises a nucleotide sequence encoding a secretion peptide.

Embodiment 20. The polynucleotide of any one of the preceding embodiments, wherein the complement C3d-binding polypeptide comprises:
(a) an Sbi domain III comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 3; or
(b) an Sbi domain IV comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 4; or
(c) both (a) and (b).

Embodiment 21. The polynucleotide of any one of the preceding embodiments, wherein the complement C3d-binding polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 5.

Embodiment 22. The polynucleotide of any one of the preceding embodiments, wherein the fusion polypeptide has at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 8.

Embodiment 23. The polynucleotide of any one of the preceding embodiments, wherein the polynucleotide has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 18.

Embodiment 24. The polynucleotide of any one of the preceding embodiments, wherein the polynucleotide has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 19.

Embodiment 25. The polynucleotide of any one of the preceding embodiments, wherein the polynucleotide has at least 80%, at least 85%, at least 90%, at least 91%, at least 92%, at least 93%, at least 94%, at least 95%, at least 96%, at least 97%, at least 98%, at least 99% or 100% identity to the nucleotide sequence of SEQ ID NO: 10.

Embodiment 26. The polynucleotide of any one of the preceding embodiments, wherein the polynucleotide is a polyribonucleotide comprising one or more modified ribonucleotides comprising: a modified nucleobase, a modified ribose, a modified backbone, or any combination thereof.

Embodiment 27. The polynucleotide of embodiment 26, wherein the one or more modified ribonucleotides each comprise a 5′ monophosphate.

Embodiment 28. The polynucleotide of embodiment 26 or 27, wherein the one or more modified ribonucleotides comprise modified ribonucleotides that comprise a N4-acetylcytidine nucleoside.

Embodiment 29. The polynucleotide of embodiment 28, wherein the polyribonucleotide comprises cytidine nucleosides, and:
(i) at least 5% of cytidine nucleosides in the polyribonucleotide comprise N4-acetylcytidine;
(ii) less than 100% of cytidine nucleosides in the polyribonucleotide comprise N4-acetylcytidine; or
(iii) at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% of cytidine nucleosides in the polyribonucleotide comprise N4-acetylcytidine.

Embodiment 30. The polynucleotide of any one of embodiments 26-29, wherein the one or more modified ribonucleotides comprise modified ribonucleotides that comprise a 5-hydroxymethyluridine nucleoside.

Embodiment 31. The polynucleotide of embodiment 30, wherein the polyribonucleotide comprises uridine nucleosides and:
(i) at least 5% of uridine nucleosides in the polyribonucleotide comprise 5-hydroxymethyluridine;
(ii) less than 100% of uridine nucleosides in the polyribonucleotide comprise 5-hydroxymethyluridine;
(iii) at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of uridine nucleosides in the polyribonucleotide comprise 5-hydroxymethyluridine; or
(iv) more than 60% of uridine nucleosides in the polyribonucleotide comprise 5-hydroxymethyluridine.

Embodiment 32. The polynucleotide of any one of embodiments 26-31, wherein the one or more modified ribonucleotides comprises a nucleoside comprising: N4-acetylcytidine, 5-hydroxymethyluridine, N1-methylpseudouridine, pyridin-4-one ribonucleoside, 5-aza-uridine, 6-aza-uridine, 2-thio-5-aza-uridine, 2-thio-uridine (s2U), 5-methyl cytidine (m5C), 5-aza-cytidine, 6-aza-cytidine, pseudoisocytidine, 3-methyl-cytidine (m3C), 5-formyl-cytidine (f5C), N4-methyl-cytidine (m4C), 2-amino-purine, 2, 6-diaminopurine, 2-amino-6-halo-purine, 6-halo-purine, inosine (I), 1-methyl-inosine (ml I), wyosine (imG), methylwyosine (mimG), 5-hydroxycytidine, 5-hydroxymethylcytidine, 5-carboxycytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N1-hydroxypseudouridine, 2′-O-methyl-N1-methylpseudouridine, N1-ethylpseudouridine, N1-hydroxymethylpseudouridine, ara-uridine, N6-methyladenosine, 2-aminoadenosine, 3-methyladenosine, 7-deazaadenosine, 8-oxoadenosine, thienoguanosine, 7-deazaguanosine, 8-oxoguanosine, 6-O-methylguanine, or any combination thereof.

Embodiment 33. The polynucleotide of any one of embodiments 26-32, wherein the one or more modified ribonucleotides comprise modified ribonucleotides that comprise a 2′-O-acetylated ribose.

Embodiment 34. The polynucleotide of embodiment 33, wherein the modified ribonucleotides comprising a 2′-O-acetylated ribose comprise modified ribonucleotides comprising a 2′-O-acetylated ribose and an adenine nucleobase.

Embodiment 35. The polynucleotide of embodiment 33 or 34, wherein the modified ribonucleotides comprising a 2′-O-acetylated ribose comprise modified ribonucleotides comprising a 2′-O-acetylated ribose and a guanine nucleobase.

Embodiment 36. The polynucleotide of any one of embodiments 33-35, wherein the modified ribonucleotides comprising a 2′-O-acetylated ribose comprise modified ribonucleotides comprising a 2′-O-acetylated ribose and a cytosine nucleobase.

Embodiment 37. The polynucleotide of any one of embodiments 33-36, wherein the modified ribonucleotides comprising a 2′-O-acetylated ribose comprise modified ribonucleotides comprising a 2′-O-acetylated ribose and a N4-acetylcytosine nucleobase.

Embodiment 38. The polynucleotide of any one of embodiments 33-37, wherein the modified ribonucleotides comprising a 2′-O-acetylated ribose comprise modified ribonucleotides comprising a 2′-O-acetylated ribose and a uracil nucleobase.

Embodiment 39. The polynucleotide of any one of embodiments 33-38, wherein the modified ribonucleotides comprising a 2′-O-acetylated ribose comprise modified ribonucleotides comprising a 2′-O-acetylated ribose and a 5-hydroxymethyluracil nucleobase.

Embodiment 40. The polynucleotide of any one of embodiments 33-39, wherein the modified ribonucleotides comprising a 2′-O-acetylated ribose comprise modified ribonucleotides comprising a 2′-O-acetylated ribose and a N1-methylpseudouracil nucleobase.

Embodiment 41. The polynucleotide of any one of embodiments 33-40, wherein:
(i) at least 5% of the ribose moieties in the polyribonucleotide are acetylated (2′-O-acetylated), or
(ii) about 5% to about 99% of the ribose moieties in the polyribonucleotide are acetylated (2′-O-acetylated).

Embodiment 42. The polynucleotide of any one of embodiments 33-41, wherein the polyribonucleotide comprises a cap structure and the cap structure does not comprise a 2′-O-acetylated ribose.

Embodiment 43. The polynucleotide of any one of embodiments 33-41, wherein the polyribonucleotide comprises a cap structure and the cap structure comprises a 2′-O-acetylated ribose.

Embodiment 44. The polynucleotide of any one of embodiments 33-43, wherein the polyribonucleotide further comprises one or more ribonucleotides that do not comprise a 2′-0 acetylated ribose.

Embodiment 45. The polynucleotide of any one of embodiments 1-44, wherein the polyribonucleotide is or comprises an RNA oligo, a messenger RNA (mRNA), a gRNA, an inhibitory RNA, an miRNA or siRNA, an antisense oligonucleotide, or a combination thereof.

Embodiment 46. A fusion polypeptide encoded by the polynucleotide of any one of the preceding embodiments.

Embodiment 47. The fusion polypeptide of embodiment 46, wherein the complement C3d-binding polypeptide comprises:
(a) an Sbi domain III comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 3; or
(b) an Sbi domain IV comprising an amino acid sequence with at least 80%, at least 85%, at least 90%, at least 91%, at least 92%, at least 93%, at least 94%, at least 95%, at least 96%, at least 97%, or 100% identity to the amino acid sequence of SEQ ID NO: 4; or
(c) both (a) and (b).

Embodiment 48. The fusion polypeptide of embodiment 46 or 47, wherein the complement C3d-binding polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 5.

Embodiment 49. The fusion polypeptide of embodiments 46-48, wherein the polypeptide comprises a secretion peptide.

Embodiment 50. The fusion polypeptide of embodiments 46-49, wherein the polypeptide comprises an amino acid sequence having at least 80%, at least 85%, at least 90%, at least 91%, at least 92%, at least 93%, at least 94%, at least 95%, at least 96%, at least 97%, at least 98%, at least 99% or 100% identity to the amino acid sequence of SEQ ID NO: 8.

Embodiment 51. An expression vector comprising the polynucleotide of any one of embodiments 1-40.

Embodiment 52. The expression vector of embodiment 51, wherein the expression vector comprises a viral vector that is a retrovirus vector, an adenovirus vector, an adeno-associated virus vector or a lentivirus vector or an RNA vector.

Embodiment 53. A composition for delivering the fusion polyribonucleotide of any one of embodiments 1-45, the polypeptide of any one of embodiments 46-50, or the expression vector of embodiment 51 or 52.

Embodiment 54. The composition of embodiment 53, wherein the composition is a pharmaceutical composition and further comprises a pharmaceutically acceptable excipient.

Embodiment 55. The composition of embodiment 54, wherein the pharmaceutical composition is or comprises an immunogenic composition, a vaccine, a gene therapy, a chemotherapy, a protein replacement therapy, an immunotherapy, an antibody therapy, an immune-modulation therapy, a cell engineering therapy, or any combination thereof.

Embodiment 56. A cell comprising the polyribonucleotide of any one of embodiments 1-45, the fusion polypeptide of any one of embodiments 46-50, or the expression vector of embodiment 51 or 52.

Embodiment 57. A method of making a polynucleotide that encodes a fusion polypeptide comprising: recombinantly joining a first nucleotide sequence that encodes an antigen, and a second nucleotide sequence that encodes a complement C3d-binding polypeptide from an immunoglobulin-binding protein (Sbi) of Staphylococcus aureus.

Embodiment 58. A method comprising administering to a subject in need thereof at least one dose of the pharmaceutical composition of embodiment 54 or 55.

Embodiment 59. The method of embodiment 58, wherein the at least one dose is administered in an effective amount to:
(i) induce an immune response against the antigen in the subject;
(ii) stimulate B cells in a subject; or
(iii) both (i) and (ii).

Embodiment 60. The method of embodiment 59, wherein B cells are stimulated without inducing a T cell response.

Embodiment 61. The method of embodiment 59, wherein B cells are stimulated in the absence of T cells.

Embodiment 62. The method of embodiment 59, wherein B cells are stimulated in the presence of T cells.

Embodiment 63. The method of embodiment 62, wherein the T cells have a reduced ability to induce an immune response.

Embodiment 64. A method for enhancing the immunogenicity of an antigen, comprising administering to an immunosuppressed subject, the polynucleotide of any one of embodiments 1-45, or the pharmaceutical composition of embodiment 54 or 55.

Embodiment 65. The method of embodiment 64, wherein enhancing immunogenicity of an antigen comprises stimulating an immune response against the antigen.

Embodiment 66. The method of embodiment 65, wherein enhancing immunogenicity of an antigen comprises stimulating a humoral immune response.

Embodiment 67. The method of embodiment 66, wherein the humoral immune response is an antibody response.

Embodiment 68. A method for stimulating an immune response against an antigen, comprising administering to an immunosuppressed subject, the polynucleotide of any one of embodiments 1-45, or the pharmaceutical composition of embodiment 54 or 55.

Embodiment 69. The method of any one of embodiments 64-68, wherein the immunosuppressed subject has received, is receiving, or will be receiving one or more transplants.

Embodiment 70. The method of any one of embodiments 64-68, wherein the immunosuppressed subject has received, is receiving or will be receiving one or more dialysis treatments.

Embodiment 71. The method of embodiment 69 or 70, wherein the immunosuppressed subject is administered one or more therapeutic agents.

Embodiment 72. The method of embodiment 69, wherein the one or more transplants is an organ transplant.

Embodiment 73. The method of embodiment 72, wherein the organ transplant comprises: a kidney transplant, a liver transplant, a heart transplant, a lung transplant, a pancreas transplant, a stomach transplant, an intestine transplant, or any combination thereof.

Embodiment 74. The method of embodiment 73, wherein the one or more transplants is a cell transplant.

Embodiment 75. The method of embodiment 74, wherein the cell transplant is a transplant of a population of stem cells (e.g., hematopoietic stem cells, induced pluripotent stem cells, or embryonic stem cells), immune cells, or any combination thereof.

Embodiment 76. The method of embodiment 74, wherein the cell transplant is a transplant of a population of bone marrow cells, blood cells, or any combination thereof.

Embodiment 77. The method of embodiment 74, wherein the cell transplant is a transplant of a population of engineered cells.

Embodiment 78. The method of embodiment 74, wherein the cell transplant is a transplant of a population of non-engineered cells.

Embodiment 79. The method of embodiment 69, wherein the one or more transplants is a tissue transplant.

Embodiment 80. The method of embodiment 79, wherein the tissue transplant comprises skin tissue transplant, bone tissue transplant, cartilage tissue transplant, adrenal tissue transplant, corneal tissue transplant, or any combination thereof.

Embodiment 81. The method of any one of embodiments 69-80, wherein the transplant is an allogeneic transplant.

Embodiment 82. The method of any one of embodiments 69 or 74-79, wherein the transplant is an allogeneic transplant.

Embodiment 83. The method of embodiment 64-68, wherein the immunosuppressed subject has one or more disorders.

Embodiment 84: The method of embodiment 83, wherein the one or more disorders comprises: a rare disease, lung disease, an autoimmune disease, liver disease, kidney disease, a cardiovascular disease, a blood disorder, a neurologic disease, an immunodeficiency disorder, a cancer, or any combination thereof.

Embodiment 85. The method of any one of embodiments 69-84, wherein the method stimulates a humoral immune response.

Embodiment 86. The method of embodiment 85, wherein the humoral response is an antibody response.

Embodiment 87. The method of any one of embodiments 58-86, wherein administration of the pharmaceutical composition or the polynucleotide results in an increased antibody response.

Embodiment 88. The method of embodiment 87, wherein the increased titer of the antibody response is compared to administration of an otherwise similar composition that does not comprise a polynucleotide comprising an Sbi domain III, or a fragment or variant thereof; and/or an Sbi domain IV, or a fragment or a variant thereof.

Embodiment 89. The method of embodiment 88, wherein the antibody response is increased by at about 1.5-fold to about 500-fold.

Embodiment 90. The method of any one of embodiments 64-89, wherein the immunosuppressed subject has received or is receiving an immunosuppressive therapy.

Embodiment 91. The method of embodiment 90, wherein the immunosuppressive therapy comprises: an organ transplant conditioning regimen, a therapy that suppresses an immune system (e.g., an antibody therapy), a B-cell targeting therapy, a T-cell targeting therapy, a chemotherapy, a radiation therapy, a cancer therapy, a treatment for an inflammatory disease and/or a treatment for an autoimmune disease.

Embodiment 92 The method of embodiment 91, wherein the B cell targeting therapy is or comprises rituximab or belimumab.

Embodiment 93. The method of embodiment 91, wherein the organ transplant conditioning regimen comprises a calcineurin inhibitor, an antiproliferative agent, a steroid, an mTOR inhibitor, or any combination thereof.

Embodiment 94. The method of embodiment 93, wherein the calcineurin inhibitor comprises tacrolimus.

Embodiment 95. The method of embodiment 93, wherein the antiproliferative agent comprises mycophenolate mofetil.

Embodiment 96. The method of embodiment 93, wherein the steroid comprises prednisone.

Embodiment 97. The method of embodiment 91, wherein the organ transplant conditioning regimen comprises tacrolimus, mycophenolate mofetil and prednisone, or any combination thereof.

Embodiment 98. A composition comprising the polynucleotide of any one of embodiments 1-45, or the pharmaceutical composition of embodiment 54 or 55, for use in: enhancing the immunogenicity of an antigen, or for stimulating an immune response against an antigen.

Embodiment 99. Use of a composition comprising the polynucleotide of any one of embodiments 1-45, or the pharmaceutical composition of embodiment 54 or 55, in the preparation of a medicament for enhancing the immunogenicity of an antigen, or for stimulating an immune response against an antigen.

Embodiment 100. The composition for use of embodiment 98, or the use of embodiment 88, wherein the polynucleotide or pharmaceutical composition is administered to a subject.

Embodiment 101. The composition for use of embodiment 98, or the use of embodiment 88, wherein the subject is an immunosuppressed subject.

Embodiment 102. The method of embodiment 101, wherein the immunosuppressed subject has received, is receiving, or will be receiving one or more transplants.

Embodiment 103. The embodiment of 101 or 102, wherein the immunosuppressed subject has received, is receiving or will be receiving one or more dialysis treatments.

Embodiment 104. The method of embodiment 102 or 103, wherein the immunosuppressed subject is administered one or more therapeutic agents.

Embodiment 105. The method of embodiment 102, wherein the one or more transplants is an organ transplant.

Embodiment 106. The method of embodiment 105, wherein the organ transplant comprises: a kidney transplant, a liver transplant, a heart transplant, a lung transplant, a pancreas transplant, a stomach transplant, an intestine transplant, or any combination thereof.

Embodiment 107. The method of embodiment 102, wherein the one or more transplants is a cell transplant.

Embodiment 108. The method of embodiment 107, wherein the cell transplant is a transplant of a population of stem cells (e.g., hematopoietic stem cells, induced pluripotent stem cells, or embryonic stem cells), immune cells, or any combination thereof.

Embodiment 109. The method of embodiment 108, wherein the cell transplant is a transplant of a population of bone marrow cells, blood cells, or any combination thereof.

Embodiment 110. The method of embodiment 107, wherein the cell transplant is a transplant of a population of engineered cells.

Embodiment 111. The method of embodiment 107, wherein the cell transplant is a transplant of a population of non-engineered cells.

Embodiment 112. The method of embodiment 102, wherein the one or more transplants is a tissue transplant.

Embodiment 113. The method of embodiment 112, wherein the tissue transplant comprises skin tissue transplant, bone tissue transplant, cartilage tissue transplant, adrenal tissue transplant, corneal tissue transplant, or any combination thereof.

Embodiment 114. The method of any one of embodiments 102-113, wherein the transplant is an allogeneic transplant.

Embodiment 115. The method of embodiment 101, wherein the immunosuppressed subject has one or more disorders.

Embodiment 116: The method of embodiment 115, where one or more disorders comprises: a rare disease, lung disease, an autoimmune disease, liver disease, kidney disease, a cardiovascular disease, a blood disorder, a neurologic disease, an immunodeficiency disorder, a cancer, or any combination thereof.

Embodiment 117. The method of any one of embodiments 58-97, the composition for use of embodiment 98, or the use of embodiment 99, wherein a single dose, or a plurality of doses of the polynucleotide or pharmaceutical composition is administered to the subject.

Embodiment 118. The method of embodiment 117, the composition for use of embodiment 117, or the use of embodiment 117, wherein a first dose and one or more subsequent doses are in the same amount amounts.

Embodiment 119. The method of embodiment 117, the composition for use of embodiment 117, or the use of embodiment 117, wherein a first dose and one or more subsequent doses are in different amount amounts.

Embodiment 120. The method of embodiment 117, the composition for use of embodiment 117, or the use of embodiment 117, wherein a first dose and one or more subsequent doses are administered by at least 3 days, at least 4 days, at least 5 days, at least 6 days, at least 7 days, at least 2 weeks, at least 3 weeks, or at least 4 weeks apart.

Embodiment 121. The method of embodiment 117, the composition for use of embodiment 117, or the use of embodiment 117, wherein the pharmaceutical composition is administered in combination with one or more therapeutic compositions, e.g., standard of care.

Embodiment 122. The method of any one of embodiments 58-97, the composition for use of embodiment 98, or the use of embodiment 99, wherein the subject is a mammal.

Embodiment 123. The method of embodiment 122, the composition for use of embodiment 122, or the use of embodiment 122, wherein the subject is a human.

### EXEMPLIFICATION

### Example 1: Materials and Methods Used in Examples 2-5 Disclosed Herein

gBlock amplification: Each gBlock template was amplified with T7-AGG_fwd (gaattTAATACGACTCACTATAAGGcttgttctttttgcagaagc)(SEQ ID NO: 11) and 120 pA_rev (TTTTTTTTTTTTTTTTTTTTTTTTTTTTTTTTTTTTTTTTTTTTTTTTTTTTTTTTTTTTTTTTTTTTTTTTTTTTTTTTTTTTTTTT TTTTTTTTTTTTTTTTTTTTTTTTTTTTTTTTagaatgtgaagaaactttctttttattag) (SEQ ID NO: 12) using Herculase II polymerase (Agilent) with an annealing temperature of 50° C.

RNA synthesis: PCR products were cleaned up using a 0.8× ratio of SPRISelect beads to PCR reaction volume. 19.9 μL transcription mixes consisting of 1× HiScribe T7 High Yield buffer (NEB), 7.5 mM of each NTP, 7.5 mM CleanCap AG (TriLink Biotech), 2M betaine (ThermoSci), 20 mM MgCl2, and 0.1 μL/μL HiScribe T7 Polymerase Mix were added to 2.1 μL DNA solution consisting of 200 ng T7 template in nuclease free H2O. Transcription was carried out for 1 hr at 50° C. The transcribed RNAs were purified using the 500 μg capacity Monarch RNA Cleanup Kit, treated with DNAse I, and purified again using 500 μg-capacity Monarch columns. RNAs were then treated with Alkaline Phosphatase (Millipore) for 10 min at 37° C. and purified using 500 μg Monarch columns. Concentrations were determined using a NanoDrop spectrophotometer.

LNP formulation: Formulations of RNA in lipid nanoparticles (RNA-LNPs) were prepared using the NanoAssemlr Ignite microfluidic mixer (Precision Nanosystems). GenVoy-ILM lipid mixture (Precision Nanosystems) was diluted to 12.5 mM in anhydrous ethanol and combined with an aqueous solution of RNA (0.14 mg/mL) in PNI buffer (Precision Nanosystems), using the manufacturer-recommended formulation parameters. Formulations were immediately diluted 30:1 in phosphate-buffered saline (pH 7.4) and concentrated using Amicon centrifugation filters (MilliporeSigma UFC901008). Formulations were stored at 4° C. and used for in vivo studies within 14 days.

Animal vaccination: All animal experiments were carried out in accordance with the guidelines set forth by Charles River Accelerator Development Lab (CRADL) and were approved by the CRADL Institutional Animal Care and Use committee. Female BALB/C mice (7-9 weeks old) were purchased from Charles River Laboratories and housed at CRADL. Mice were acclimated for at least 3 days before the initiation of a study. On Day 1, mice were injected in the right quadriceps with 50 μL RNA-LNP formulation (10 μg RNA dose was used unless stated otherwise). For experiments involving two vaccinations, mice were additionally injected in the left quadriceps with 50 μL RNA-LNP formulation for booster immunizations. Mice were euthanized at day 10-21 (as indicated), at which time blood was collected via intracardiac stick and spleens were dissected and collected for processing. Serum was separated from blood in MiniCollect serum separator tubes (Greiner Bio-One 450472) by centrifugation at 4° C., 1200×g, for 10 minutes. Fresh serum was stored at 4° C. and used to evaluate immunogenicity by ELISA and neutralization assay, the remainder was aliquoted and frozen at −80° C.

Spike titer ELISA assay: An ELISA protocol was adapted from one previously established by Amanat, et al. (Nat Med 26: 1033-1036, 2020). Briefly, 96-well Immulon 4 HBX plates (Thermo Fisher Scientific) were coated with 50 μl per well of a 2 μg/ml solution of SARS-CoV-2 (2019-nCoV) Spike S1+S2 ECD-His recombinant protein (Sino Biological #40589-V08B1) in PBS at 4° C. overnight. Plates were washed three times with 300 μl of 0.1% Tween 20 in PBS (PBS-T), then were blocked for 1 h with 100 μl per well of 3% non-fat milk in PBS-T. Serial dilutions of serum and antibody controls were prepared in 1% non-fat milk in PBS-T, and 100 μl of each was added to the plates for 2 h at room temperature. The wells then were washed three times in PBS-T as before. Wells were then incubated in 100 μl of a 1:3,000 dilution of goat anti-mouse IgG horseradish peroxidase-conjugated secondary antibody (Sigma) in 1% milk PBS-T room temperature for 1 hour. Plates were again washed three times in PBS-T. 100 μl SIGMAFAST OPD (Sigma-Aldrich) solution was added to each well for 10 min at room temperature for 10 min. Reactions were stopped by addition of 50 μl per well of 3 M hydrochloric acid. Optical density was measured at 490 nm using a GloMax Discover (Promega) plate reader. End-point titers were determined by taking the last dilution before the signal dropped below 1 standard deviation above the average of the signal from the untreated control serum at the same dilution. The last dilution was taken as the titer if the signal never dropped below this threshold. If no signal above the threshold was detected, the value in the dilution series before the least-dilute sample tested was used. A chimeric monoclonal antibody reactive to the RBD of both SARS-CoV-1 and SARS-CoV-2, and a SARS-CoV-1 reactive mouse monoclonal antibody were used as positive and negative controls, respectively.

Pseudotype neutralization assay: Human ACE2-overexpressing HEK cells (Integral Molecular) used for viral transduction experiments were maintained in high glucose GlutaMAX-containing DMEM (ThermoFisher Scientific 10564) supplemented with 1 μg/mL puromycin, 10% heat-inactivated fetal bovine serum and 100 U/mL penicillin/streptomycin. 4 μl (for 1:37.5 dilution) or 1 μl (for 1:500 dilution) of each serum was mixed with 35 μl spike-pseudotyped GFP-encoding reporter viral particles built using a second-generation lentiviral system (Integral Molecular, lot CG-113A) in puromycin-free culture media to a total volume of 100 μl. Virus and serum were incubated in a 96-well cell culture plate at 37° C. for 1.5 h. 20,000 freshly harvested HEK cells were then added in 50 μL puromycin-free culture media, and transduction was allowed to proceed for 3 days. The Cytation 5 Cell Imaging Multi-Mode Reader and Gen5 software (BioTek) were used to quantify the number of GFP-positive cells. Images were taken of each well using a 4×PL ACH objective in the GFP (469, 525) channel with the LED set to 10.0, an integration time of 100 msec, and gain set to 11.0. Images were taken at a fixed focal height 345 μm below the plate. 6 images were taken per well with automatic overlap to allow the software to stitch them together. Image preprocessing was applied using a dark background and background flattening with a rolling ball diameter of 60 μm. For cellular analysis a primary mask was applied to the processed images with a dark background and a GFP threshold of 2000. Minimum and maximum object sizes were set to 10 μm and 1000 μm, respectively. Split touching objects, fill holes in mask, include edge objects, and analyze entire image were set to ON. Using these parameters, the software calculated a cell count to give the number of GFP-positive cells per well.

ACE2:RBD inhibition assay: The ability of vaccinated sera to inhibit ACE2:RBD binding was measured essentially as described in Nie, et al. (Nat Biotech 15: 3699-3715, 2020), using the SARS-CoV-2 Surrogate Virus Neutralization Test kit (GenScript).

ELISpot Assay: For preparation of single cell suspension from spleen tissue, following euthanasia, spleens were harvested from each animal and stored in 10% FBS/Pen-Strep/25 mM HEPES/RPMI 1640/GlutaMAX buffer. Spleens from each treatment group (n=5) were pooled together for processing. Spleen dissociation was carried out using MACS Spleen Dissociation kit and the gentleMACS OctoDissociator (Miltenyi Biotec) according to manufacturer protocol. Resulting splenocyte suspensions were then briefly centrifuged, applied to a 70 uM filter (Corning 431751), and thoroughly washed with RT 1×PBS. Suspensions were again centrifuged RT at 300 rpm for 10 minutes, aspirated, and finally resuspended in CTL media supplemented with 1% GlutaMAX. Splenocytes were stored at 4C until use.

ELISpot was performed using ImmunoSpot Mouse IFN-γ/IL-2 Double-Color kit (CTL). The day before splenocyte plating, Murine IFN-γ/IL-2 Capture Solution (Murine IFN-γ and Murine IL-2 Capture Antibodies) was prepared according to kit protocol. 15 μl/well of 70% ethanol was added to the ELISpot plate then immediately washed three times with 150 μl/well PBS. Before wells dried, 80 uL/well Murine IFN-γ/IL-2 capture solution was added. The plate was sealed and stored 4 C overnight.

The following day, capture solution was decanted from the plate and wells were washed with 150 uL PBS. Splenocytes were counted and adjusted to a concentration of 5 million live cells/mL (500,000 cells/well) in CTL-Test medium. Cells were kept at 37° C. in humidified incubator, 5% C02 until plating. 100 μL/well PepMix SARS-CoV-2 Spike Glycoprotein (JPT, #PM-WCPV-S-1) prepared according to manufacturer protocol was added to the plate and incubated 10-20 minutes at 37° C. prior to splenocyte plating to ensure optimal conditions for cell survival. Cells were then plated 100 μl/well using large orifice tips and immediately placed into a 37° C. humidified incubator, 5% C02, and incubated undisturbed for 24 hours.

The next day, wash buffers (1×PBS, 0.05% PBS-Tween, distilled water) and Anti-murine IFN-γ/IL-2 Detection Solution (Anti-murine IFN-γ (FITC)+10 μl Anti-murine IL-2 (Biotin) Detection Antibodies) were prepared. The ELISpot plate was washed twice with 200 uL/well PBS and twice more with 200 uL/well 0.05% PBS-Tween. 80 μl/well Anti-murine IFN-γ/IL-2 Detection Solution was added and incubated at room temperature for two hours. The plate was then washed three times with 200 μl/well 0.05% Tween-PBS, and tertiary solution (FITC-HRP+Strep-AP) was added and incubated at room temperature for one hour. The plate was again washed two times with 200 μl/well 0.05% Tween-PBS, and then two times with 200 μl/well distilled water. 80 μl/well Blue Developer Solution was then added and incubated at room temperature for 15 minutes. The reaction was stopped by gently rinsing the membrane with tap water, decanting, and repeating three times, followed by a final decanting with 200 μl/well distilled water. 80 μl/well Red Developer Solution was added and incubated at room temperature for 5-10 minutes. The reaction was stopped and the plate was decanted as described previously. The plate was then air-dried for two hours in running laminar flow biosafety cabinet. After drying, the plate was scanned and counted; IFN-γ spots appear red, IL-2 spots appear blue. ImmunoSpot Analyzer and ImmunoSpot Software were used.

Single-color spots, IFN-γ (red spot) and IL-2 (blue spot), were calculated by subtracting the number of double-color spots from the number of red spots and blue spots recognized in each well.

### Example 2: Enhanced Antigenicity of SARS-CoV-2 S Protein Domains with Sbi(III-IV) Fusions

This Example describes development of vaccines comprising antigen fragments that focus the immune response on particular regions of an antigen, e.g., conserved and/or functionally critical regions. Immunofocusing in this manner can improve the breadth and efficacy of the vaccine. Among other things, there are two technical challenges for vaccinating with antigens smaller than a protein or a protein subdomain (e.g., an antigen fragment). First, an antigen fragment typically needs to contain peptides that are efficiently presented on MHC complexes. Second, an antigen fragment should have epitopes folded into the same conformation as in the full protein.

For these experiments, BALB/c mice were administered a 10 μg dose of IM-delivered RNA encoding: (1) a Receptor Binding Domain of SARS-CoV-2 S protein (RBD); (2) an RBD joined (e.g., directly or indirectly) to a transmembrane domain of S protein (RBD-TM); (3) an exemplary fusion protein of an RBD and Sbi(III-IV); or (4) an exemplary fusion protein of an RBD and Sbi(III-IV) and an RBD joined (e.g., directly or indirectly) to a transmembrane domain of S protein (RBD-TM). A group of unvaccinated mice served as controls. At day 21 post-vaccination, IgG titers were evaluated in the blood using an ELISA. For evaluating the T cell response, spleens from 5 mice were pooled and plated to 4 wells at 500,000 cells/well. Two wells each were stimulated by either N-terminal or C-terminal S protein peptide pools (PepMix SARS-CoV-2 Spike Glycoprotein mix). Rates were calculated by pooling ELISPOT counts of the number of IFNγ/IL2 double-stained colonies across the 4 wells, and the error bars were calculated as the Poisson error of the pooled colony counts.

The results demonstrated that vaccinating with a minimal antigen joined (e.g., directly or indirectly) to a complement C3d-binding region of Sbi protein from Staphylococcus aureus (FIG. 1A) enhanced the titers of the resulting antibody response (FIG. 1B). By screening a large set of antigen presentation architectures, this strategy was extended by co-delivery of Sbi(III-IV)-joined (e.g., directly or indirectly) antigen with the same antigen joined (e.g., directly or indirectly) to a transmembrane domain (FIG. 1B). In addition to eliciting a humoral response, these RNA-based vaccines also elicited a IFNγ+IL2+ polyfunctional CD4+ response (FIG. 1C). It was also observed that fusing peptides as short as 25 residues in length to Sbi(III-IV) enabled them to elicit a meaningful antibody response (FIG. 1D). Without wishing to be bound to any particular theory, C3d can directly activate B cells through Complement Receptor 2 (CR2), and allow for induction of immune responses in the absence of CD4+ T cell signaling. Thus, this scaffold can serve as a synthetic immunological synapse, mimicking natural viral infection to drive a strong and appropriate immune response. Accordingly, in some embodiments, a fusion architecture described herein allows small antigens that lack MHC-presented peptides to elicit a meaningful humoral response.

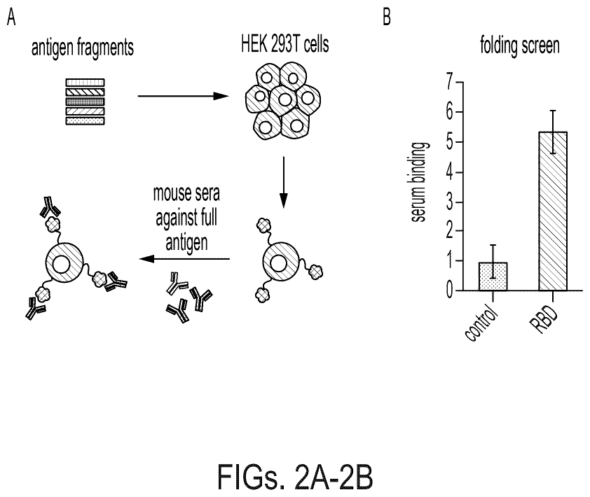

To address the challenge of structure prediction, a high-throughput screen was performed for determining whether an antigen fragment folds into the same three-dimensional conformation as the full protein (FIG. 2A). This screen included two steps. First, anti-sera against the full protein was generated by vaccinating mice with an RNA vaccine encoding a full-length protein of interest. Second, polyclonal antibodies in the sera of the mice were then used as a probe against fragments presented in the context of a mammalian surface display system. As an example, a HEK293T cell expressing a library of antigen fragments can be used in this step. Fragments that bind to antibodies generated by the full protein are likely to share conformational states with the full protein. Correctly folded fragments were identified by an increase in binding to sera antibodies (FIG. 2B). This system allowed for the screening of fragments in vitro with two orders of magnitude higher throughput than is practical in vivo.

### Example 3: Sbi(III-IV) Fusion to the RBD Domain of SARS-CoV-2 S Protein Increases Virus Neutralization

In this Example, virus neutralization activity of serum obtained from mice vaccinated with an RNA encoding an RBD, or vaccinated with an RNA encoding an Sbi(III-IV) fusion to an RBD was tested.

For this experiment, BALB/C mice were injected in the right quadriceps with 50 μL RNA-LNP formulation (10 μg RNA dose) of the indicated RNA constructs. At 10-21 days post-vaccination, serum from vaccinated mice was collected and incubated with target cells in the presence of SARS-CoV-2 virus. Serum from unvaccinated mice was used as a control. Serum samples from vaccinated mice and unvaccinated mice were subjected to a pseudotype virus neutralization assay as described in Example 1.

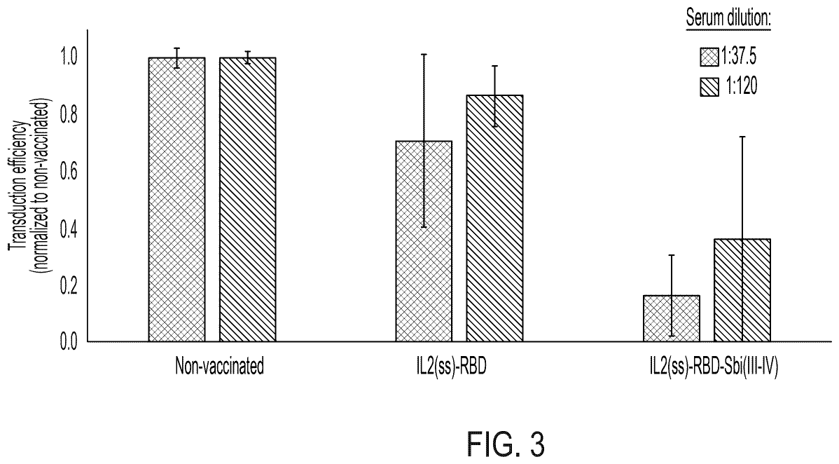

As shown in FIG. 3, fusing Sbi(III-IV) to an RBD increases pseudotype virus neutralization compared to an RBD alone. This effect was observed at both serum dilutions. These data demonstrated that vaccination with RNA encoding an Sbi(III-IV) fusion to an RBD results in an antibody response with SARS-CoV-2 virus neutralizing properties.

### Example 4: Stimulation of B Cells by an Exemplary Sbi(III-IV) Fusion to an RBD

This Example describes the stimulation of B cells in mice administered a low dose of an RNA encoding an Sbi(III-IV) fusion to an RBD.

For this experiment, BABL/C mice were vaccinated with a 0.5 μg, 2.5 μg or 10 μg IM-dose of an RNA comprising a construct having an IL2 secretion peptide and an exemplary fusion polypeptide with an RBD joined (e.g., directly or indirectly) to Sbi(III-IV), also referred to as IL2(ss)-RBD-Sbi(III-IV). At 10-21 days post-vaccination, blood and spleen were collected from the mice. For titer evaluation, blood from the animals were analyzed with an ELISA assay. For evaluation of T cell responses, an ELISPOT detecting IFNg and/or IL-2 was used.

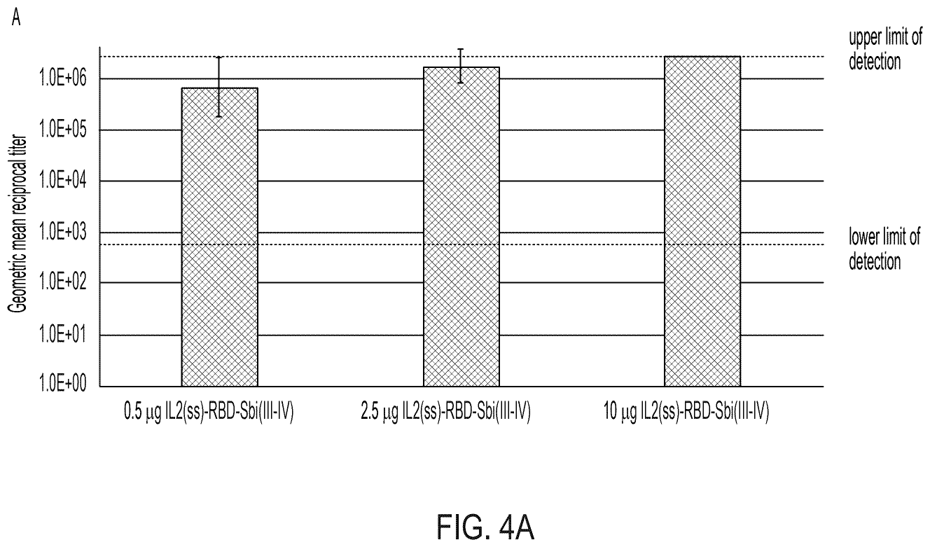

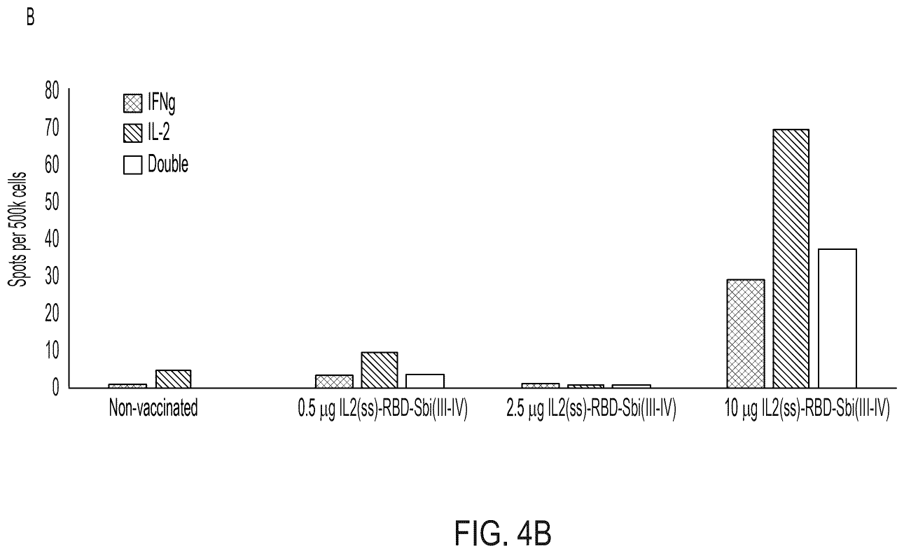

As shown in FIG. 4A, high titers were observed when RBD-Sbi(III-IV) dose was decreased from 10 μg per mouse to 0.5 μg per mouse. Reducing the dose of IL2(ss)-RBD-Sbi(III-IV) below 10 μg reduces the cellular response (FIG. 4B). These data suggested that the antibody response elicited by vaccination with IL2(ss)-RBD-Sbi(III-IV) involves bypassing T cells by directly stimulating B cells.

### Example 5: Priming Followed by Boost Increases Antibody Responses Elicited by an Exemplary Sbi(III-IV) Fusion to an RBD

In this Example, the effect of a vaccination regimen comprising a priming dose followed by a booster dose was evaluated.

For this experiment, BABL/C mice in 4 groups were injected with a 50 μL RNA-LNP formulation (10 μg RNA priming dose) of an RNA comprising a construct having an IL2 secretion peptide and an exemplary fusion polypeptide with an RBD joined (e.g., directly or indirectly) to Sbi(III-IV), also referred to as IL2(ss)-RBD-Sbi(III-IV). One group of mice did not receive additional administration of the vaccine (no boost group). The other three groups of mice received a booster dose of 50 μL RNA-LNP formulation (10 μg RNA dose) at days 3, 7 and 14 respectively. At 10-21 day post-vaccination, blood and serum was collected from the mice and subjected to an ELISA assay as described in Example 1 for determination of RBD antibody titer.

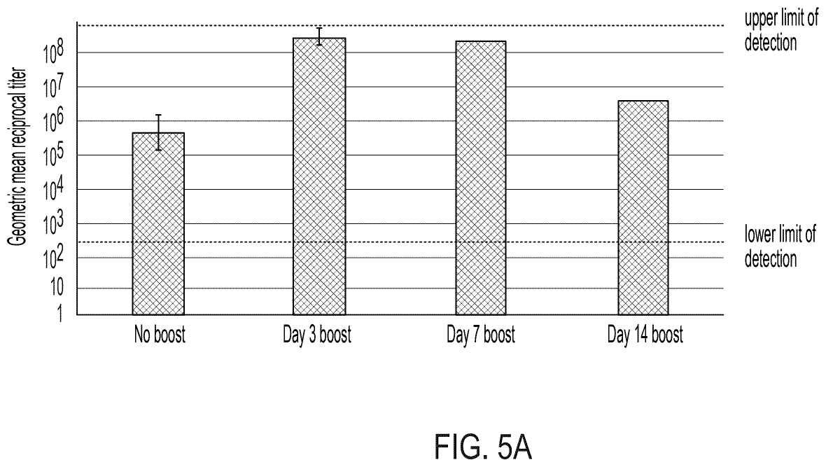

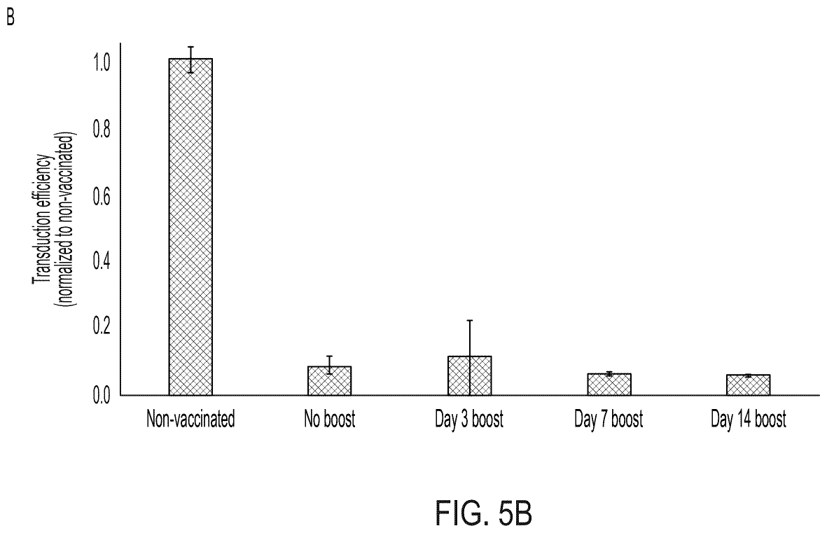

FIG. 5A shows that administration of a booster dose increased the titer generated by IL2(ss)-RBD-Sbi(III-IV) by a factor of ≥776 (when done on day 3 after the prime), by a factor of 588.1 (when done on day 7 after the prime), and by a factor of ≥9.2 (when done on day 14 after the prime). FIG. 5B shows that both boosted and prime-only vaccinations blocked ACE2:RBD interaction in vitro at a 1:20 serum dilution. These data demonstrated that the antibody response elicited by vaccination with IL2(ss)-RBD-Sbi(III-IV) can be enhanced with a vaccination regimen comprising a priming dose and a booster dose.

In the next experiment, the effect of different priming doses on antibody titer was tested. BALB/C mice were injected with a 1 μg or 5 μg priming dose of an RNA-LNP formulation comprising an RNA construct having a Spike protein secretion peptide and an exemplary fusion polypeptide with an RBD joined (e.g., directly or indirectly) to Sbi(III-IV) (S(ss)-RBD-Sbi(III-IV)) or an RNA comprising a construct having a Spike protein secretion peptide and an (S(ss)-RBD). The mice received a booster dose on day 4 after priming. At 10-21 day post-vaccination, blood and serum was collected from the mice and subjected to an ELISA assay as described in Example 1 for determination of RBD antibody titer.

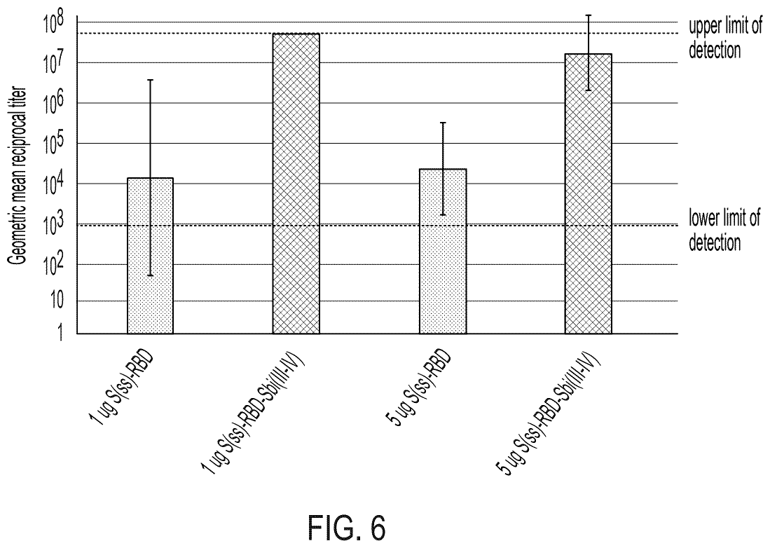

FIG. 6 shows that priming with 1 μg or 5 μg of an RNA comprising S(ss)-RBD-Sbi(III-IV) resulted in a similar antibody titers. These data demonstrated that a lower priming dose is sufficient to induce an antibody response.

### Example 6: Functional Validation of Immunosuppressed Mouse Model and Improved Cellular Responses Following Vaccination with Sbi Fusion

This Example describes the generation and validation (including functional validation) of an immunosuppressed (IS) mouse model that can be used as a surrogate for human subjects who are receiving immunosuppressants, have a weakened immune system, and/or are transplant recipients.

This Example also describes use of the IS mouse model in evaluating cellular immune responses after vaccination with an LNP formulation comprising an RNA encoding an exemplary fusion polypeptide with an Sbi (III-IV) joined (e.g., directly or indirectly) to an RBD.

### Methods

Immunosuppression formulation and dosing: Once a week, Tacrolimus (Sigma, 1642802) was prepared at 1 mg/mL in 10% DMSO saline solution. Mycophenolate Mofetil (Ascend Laboratories, LLC, NDC 0054-3722-63 provided by Northeast Medical, RX 10146014) and prednisone (Hikma Pharmaceuticals USA, Inc, NDC 67877-230-22 provided by Northeast Medical, RX 10037438) were supplied at 200 mg/mL and 1 mg/mL, respectively. Tacrolimus, Mycophenolate Mofetil, and Prednisone were combined to create an immunosuppressive drug cocktail (also referred to herein as “TMP”). Before each dosing, the immunosuppressive drug cocktail was prepared in saline and given in a total volume of 200 μL, such that the mice received a dose of 4 mg/kg Tacrolimus, 30 mg/kg Mycophenolate Mofetil, and 4 mg/kg Prednisone at each dose.

Vaccine Formulation: Vaccine treatment was diluted in sterile saline immediately prior to use in a Biosafety Cabinet and mixed gently but thoroughly by pipetting up and down slowly several times. Remaining thawed bulk vaccine test article and remaining vaccine test articles were stored under refrigerated conditions (2 to 8° C.) and shipped on cold packs following injection for analysis.

In Vivo Methods: All animal experiments were carried out in accordance with animal use protocol AUP #23-0724-MR. The study was designed to: (1) evaluate physical and physiological characteristics in immunosuppressed mice as compared to immunocompetent mice; and (2) evaluate the immune responses to vaccination in immunocompetent or immunosuppressed mice.

Twelve female and twelve male Balb/C mice, 7-9 weeks old, were purchased from Charles River Laboratory and housed at Aragen. Mice were provided standard feed and water ad libitum, with a 12-hour light/dark cycle. The animals were acclimatized for at least 3 days prior to initiation of the study. Prior to the study initiation, mice were weighed and re-distributed in 8 groups of 3 such that the weight distribution and average weight of animals in each group was comparable.

A subset of mice (n=6 males and n=6 females) were started on TMP via oral gavage, on Day 1, three days prior to immunization. Treatment was continued daily throughout the study until Day 37. Control mice were administered PBS via oral gavage.

On Day 3 and Day 24, mice in Groups 5-8 were immunized with the exemplary vaccine. Immunophenotyping by FACS was carried out on Day 3, Day 17, and Day 38. Clinical observations were collected throughout the study. Serum was collected to evaluate the humoral response and the cellular immune response was determined by ELISPOT on Day 38.

Clinical Observations and Body Weights: All mice were observed twice daily for the duration of the study. Body weights were taken on the day prior to vaccination and then daily for 3 days, followed by weekly body weights until the end of the study. Body weights were taken with a rectal probe prior to day 0 and then multiple times on immunization Day 3 and Day 6; pre-dose, 6 h and 24 h post dose. Food consumption was monitored weekly, in which food trays were weighed before and after cage changes for an estimate of food consumed by cage.

Injection sites were also monitored for erythema and edema on Day 3 and Day 24 at pre-dose and at 24, 48 and 72-hours post dose and weekly (Day 10, Day 17, Day 31). Injection sites were shaved as necessary to observe the skin.

Vaccination and sample collection: Injection sites were shaved 1 to 2 days prior to the injection. On Day 3, Groups 5-8 received a prime injection of 50 μL administered intramuscularly in the left quadriceps. On Day 24, Groups 5-8 received a boost injection of 50 μL administered intramuscularly in the right quadriceps. Control groups (Groups 1-4) were dosed with sterile PBS.

On Day 3 and Day 17, blood was collected via retro orbital bleeds (approximately 150 μL) from animals placed under inhaled isoflurane anesthesia on Day 3 for Groups 1-4 and on Day 17 for Groups 1-8. Blood was collected into K3/EDTA tubes and rocked until processed for FACS analysis.

On Day 35, mice were bled via cardiac exsanguination while heavily anesthetized with inhaled isoflurane, followed by cervical dislocation to ensure euthanasia. Blood was split between K3/EDTA tubes (150 μL for FACS analysis) and serum collection tube for serum preparation.

The following organs were collected from each animal and weighed: Spleen, thymus, liver, lungs, kidneys, adrenal gland and lymph nodes (inguinal and popliteal). All tissues, except for the spleen, were collected into NBF and processed into paraffin blocks. For Group 1, Group 3, Group 5-8, the spleens were placed into 1 mL RPMI media in 1.5 ml tubes and stored on ice until processing for ELISPOT assays. The remaining spleens were placed into neutral buffered formalin (NBF) and processed into paraffin blocks for histological analysis.

FACS Analysis: Immunophenotyping by FACS was performed on Day 3, Day 17, and Day 38, using approximately 150 μL of blood for each sample in K2EDTA tubes. Red blood cells (RBC) were lysed by incubating with 10 mL of RBC lysis buffer (i.e., ACK buffer) for 5 minutes at room temperature (RT) then centrifuged at 300×g for 5 minutes. The samples were washed with 5 mL of PBS with 0.5% BSA (FACS buffer). Single cell suspensions of RBC-lysed samples were aliquoted into 96-well round bottom plates (Falcon, Cat #351190), with approximately ⅔ of each sample volume allocated for staining with surface antibodies shown in Table 4 and the remaining ⅓ allocated for staining with Hoechst dyes shown in Table 5.

Cells were washed with PBS and stained with viability dye (Zombie-Aqua, 1:1000 dilution in PBS) for 15-20 min at 4° C., followed by 1× wash with FACS buffer. For each wash step, the plate(s) were centrifuged at 300×g at 4° C. for 5 minutes and the supernatant decanted. The samples were incubated with Fc blocking antibody at RT for 10 minutes and then stained with a mix of anti-mouse antibodies to cell surface markers (in 100 μL FACS buffer per well) for 20 minutes on ice. For Panel 1, (Table 4), samples were washed 2× with FACS buffer and resuspended in 220 μL of FACS buffer and 190 μL of each sample was acquired on the Attune NxT flow cytometer (Thermo Fisher Scientific). For Panel 2 (Table 5), samples were washed 1× with FACS buffer, fixed and permeabilized (ThermoFisher Scientific #00-5521-00) for 20 minutes. Samples were then washed 2× with permeabilization buffer (ThermoFisher Scientific, #00-8333-56) and incubated with anti-Ki67 antibody in 100 μL permeabilization buffer at room temperature for 30 minutes. Cells were then washed and incubated with PBS containing 2 μg/mL Hoechst 33342 Ready Flow Reagent (ThermoFisher Scientific, #R37165) for 15 minutes at room temperature. Following a final wash, cells were resuspended in 220 μL of PBS and 190 μL of each sample was acquired with a flow cytometer.

TABLE 4
Panel 1 antibodies.

| | | Attune | BioLegend | Dilution/ |
|---|---|---|---|---|
| Number | Marker/Reagent | Channel | Catalogue # | Concentration |
| 1 | Live dead aqua | VL2 | 423102 | 1:1000 |
| 2 | CD45-PerCP Cy5.5 | BL3 | 103132 | 1:200 |
| 3 | CD3-AF647 | RL1 | 100209 | 1:100 |
| 4 | CD4-AF700 | RL2 | 100536 | 1:200 |
| 5 | CD8a-AF488 | BL1 | 100723 | 1:200 |
| 6 | CD19-PECy7 | YL4 | 152418 | 1:100 |
| 7 | CD49b-PE | YL1 | 103506 | 1:100 |
| 8 | GR1-BV711 | VL4 | 108443 | 1:300 |
| 9 | NKp46-BV421 | VL1 | 137612 | 1:100 |

TABLE 5
Panel 2 antibodies.

| Number | Marker/Reagent | Attune Channel | Catalogue # | Dilution/ |
|---|---|---|---|---|
| 1 | Live dead aqua | VL2 | 432102 | 1:1000 |
| 2 | CD3-AF647 | RL1 | 100209 | 1:100 |
| 3 | CD4-AF700 | RL2 | 100536 | 1:200 |
| 4 | CD8a | BL1 | 100723 | 1:200 |
| 5 | Ki67 | YL1 | 652404 | 1:100 |
| 6 | Hoechst 33342 | VL1 | 00-8333-56 | 2 μg/mL |
| | Ready Flow | | | |

ELISPOT Assay: Spleens were dissociated through a 70 micron strainer using 10 mL 1×RPMI-1640 (Gibco, REF:22400-071)+2% heat inactivated FBS (hiFBS) (Atlas Biologicals, Cat #: FS-0500-AD). Cells were centrifuged at 400×g for 5 min at 10° C. and the supernatant was decanted. The RBCs were lysed with 2 mL of 1×BD Pharm-Lyse (BD Biosciences, Cat #: 555899) by pipetting up and down serval times. Within 5 minutes, the lysis reaction was neutralized with 30 mL with RPMI+2% hiFBS and the samples were centrifuged at 400×g for 5 minutes at 10° C. and decanted. The splenocytes were resuspended in complete culture medium (RPMI-1640+10% HI-FBS+1× antibiotic-antimycotic solution+1×2 Mercaptoethanol (Gibco, REF: 21985-023) and strained over a 70 micron strainer. Cell concentration was determined with a hemocytometer and adjusted to 5 million cells per mL.

ELISPOT, 2-color for IFNγ+IL-5, was performed to confirm cellular response to the vaccine and analyses Th1 (IFNg) and Th2 (IL-5) responses (MabTech, FluoroSpot Plus: Mouse IFN-γ/IL-5 FSP-4143-2). Cells (2.5×105 cells/well) were stimulated in triplicate with either CM plus anti-CD28 antibody or complete culture medium with 5 μg/mL of a SARS2-RBDMUT10-1 peptide pool (JPT Peptide Technologies) plus the anti-CD28 antibody in triplicate for 48 hours. The plates were stained according to the manufacturer's protocol and then the number of Thi (IFNγ) and Th2 (IL-5) cells were determined.

SARS COV-2 MN V Assay: The SARS CoV-2 MN V is a cell-based assay that is designed to determine the ability of SARS CoV-2 neutralizing antibodies to inhibit the infection of 293T-ACE2 cells by SARS CoV-2 Reporter Virus Particles (RVP) that express green fluorescent protein (GFP). Serum samples were pre-incubated with a known quantity of SARS CoV-2-GFP for 60 (t 5) minutes prior to infection of 293T-ACE2 cells. COVID-19 infection was monitored 48 (±4) hours following infection by counting the number of green, fluorescent cells using the Cytation 5 cell imaging reader.

Samples were analyzed using a microneutralization assay for detection of SARS CoV-2 neutralizing antibodies. Applicable critical reagents used for sample testing are described in Table 6. In this method, samples were assessed in duplicate for virus neutralization at dilutions 1:50, 1:150, 1:450, 1:1,350, 1:4,050 and 1:12,150.

TABLE 6
Reagents used for sample testing.

| | | | | Material | Storage | Working |
|---|---|---|---|---|---|---|
| Compound | Purpose | Source | Lot | Conc. | Conditions | Dilutions |
| SARS CoV-2 | Reporter | Integral | CG- | 2.49 × 106 | −90° C. | 1:64 (Final, |
| RVP-GFP | Virus | Molecular | 376A | TU/mL | to −60° C. | in-well) |
| | Particle | | | | | |
| Positive | Control | Sino | MB17E | Pre-diluted | −90° C. | 1:50 (Final, |
| Control | | Biological | 16901 | 1:100 | to −60° C. | in-well) |

### Results

The IS mouse model described herein was developed with the aim of mimicking patients treated with immunosuppressive drug cocktails. The IS mice received TMP which consists of: 4 mg/kg Tacrolimus+30 mg/kg Mycophenolate Mofetil+4 mg/kg Prednisone, which is similar to immunosuppressive drug cocktails administered to patients, e.g., those who will be receiving a transplant. For a description of immunosuppressive drug cocktails typically administered to transplant recipients, see: https://www.nhsbt.nhs.uk/organ-transplantation/kidney/living-with-a-kidney-transplant/kidney-transplant-medicines/.

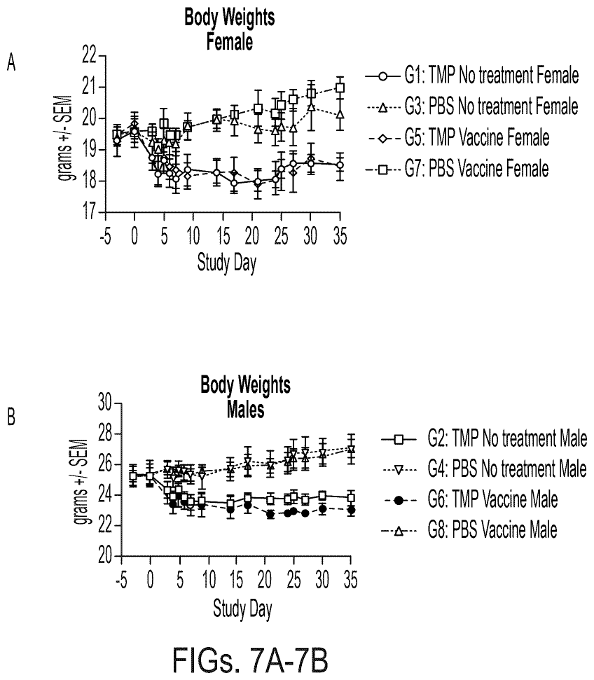

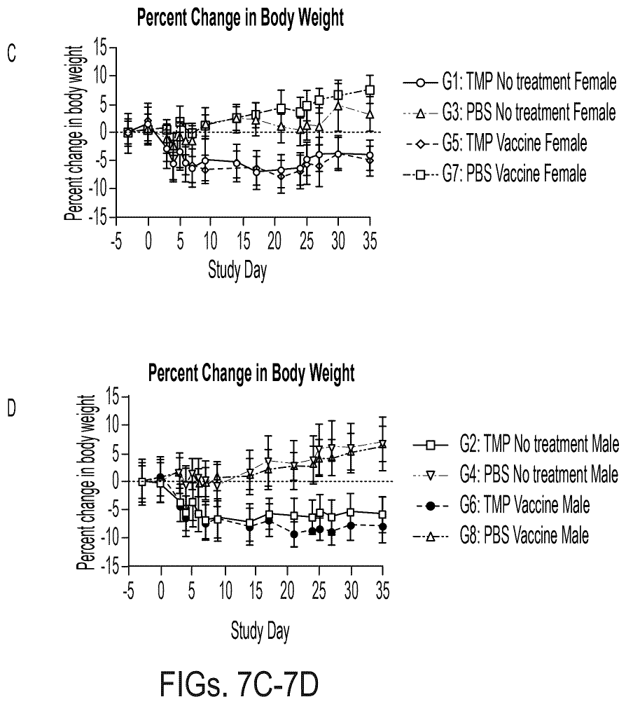

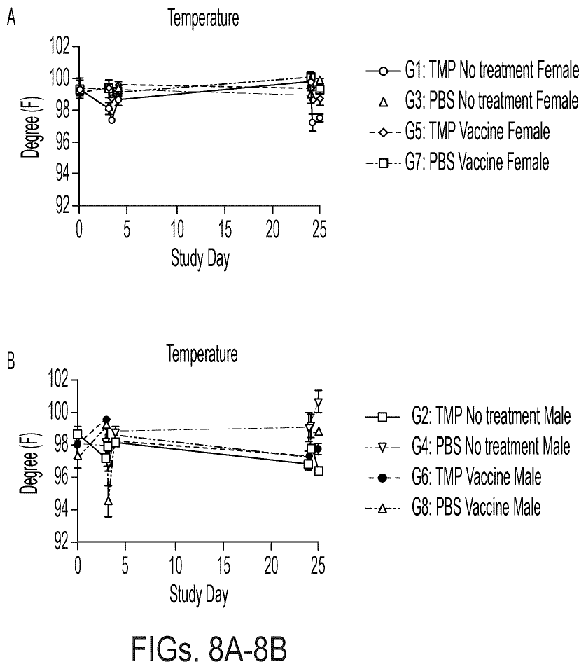

As demonstrated herein, the IS mouse model exhibited clinical features consistent with immunosuppressed patients. Immunosuppression resulted in a body weight loss in the range of 3% to 10% for both male and female mice that occurred over the first seven days of treatment but then remained stable over the remainder of the study (FIGS. 7A-7D; compare “no treatment” TMP and PBS groups). Body temperature did not change depending on whether or not the animals were immunosuppressed (FIGS. 8A-8B).

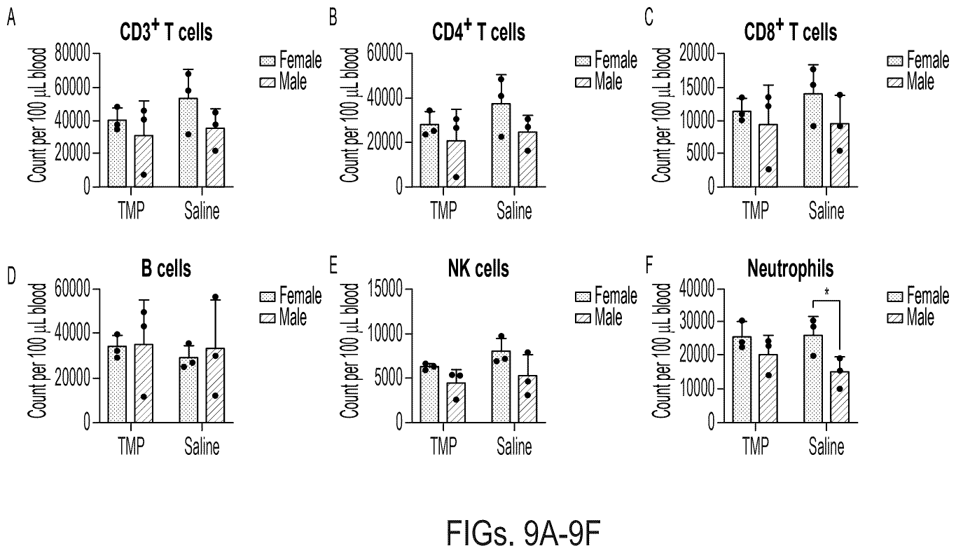

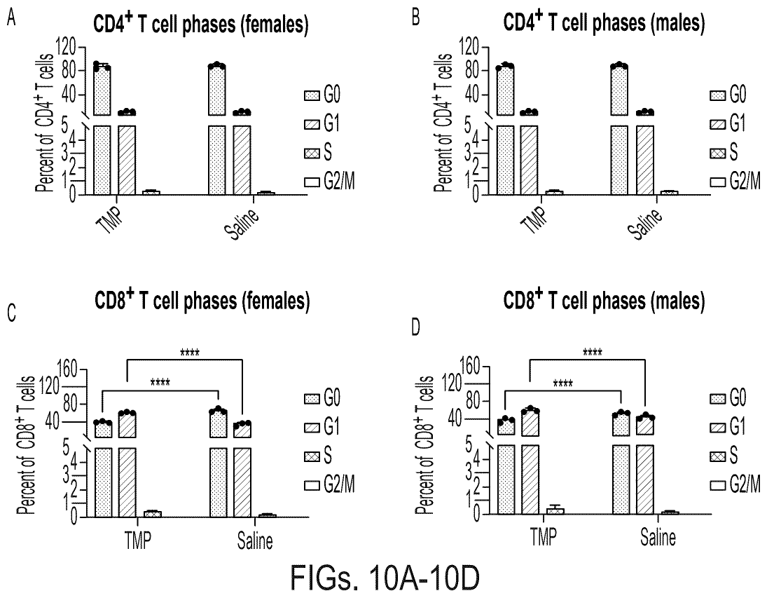

Immunophenotyping by FACS on Day 3, Day 17, and Day 38 demonstrated that daily oral administration of TMP, significantly dampened cellular immune responses in male and female BALB/c mice beginning about 2 weeks (starting on Day 17) after administration. On Day 3, TMP treatment showed no effect on counts of CD3+ total T cells, CD4+ T cells, CD8+ T cells, B cells, natural killer (NK) cells and neutrophils, relative to control mice administered saline (FIGS. 9A-9F). TMP-treated and control mice showed a similar proliferation profile of CD4+ T cells (FIGS. 10A-10D). However, as compared to control animals (saline treatment), TMP treated animals had a decreased percentage of CD8+ T cells in the G0 phase of the cell cycle and increased CD8+ T cells in the G1 phase, while percentages in S and G2/M phases were not affected (FIGS. 10A-10D).

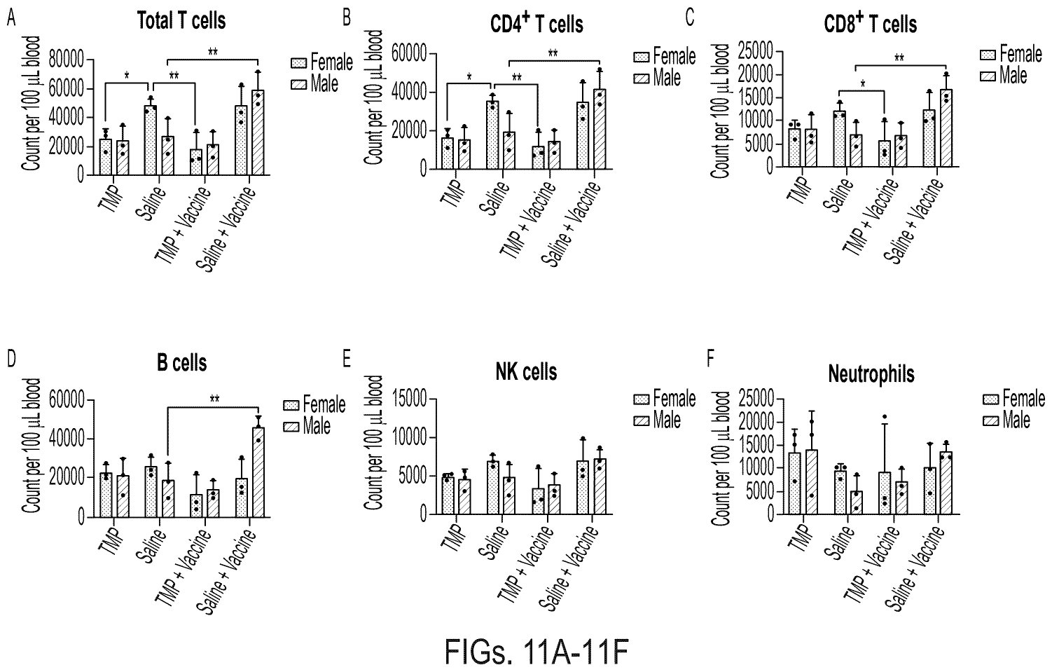

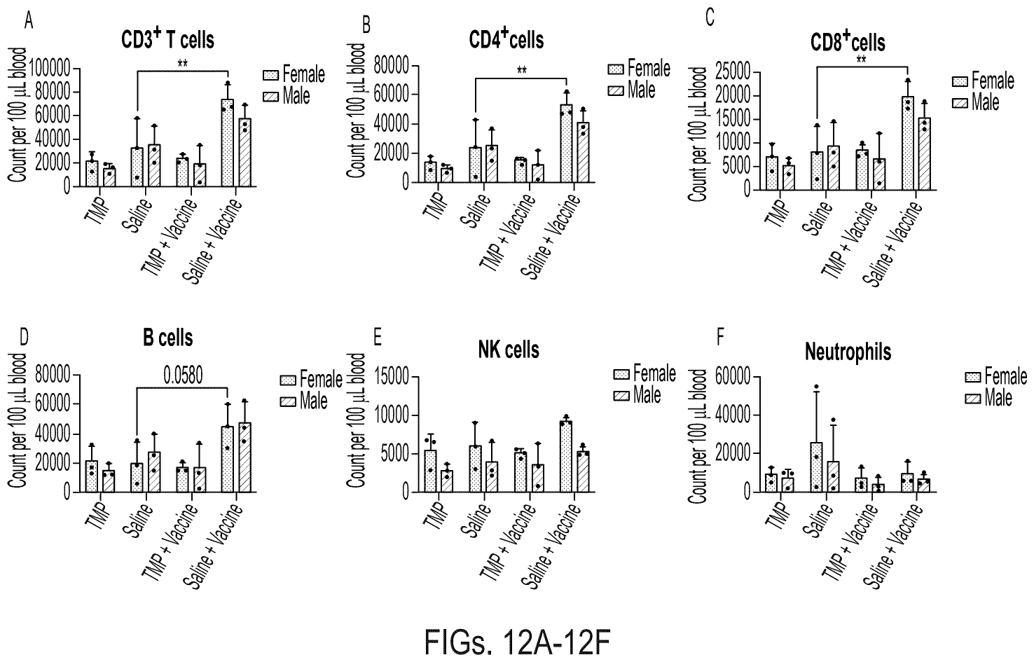

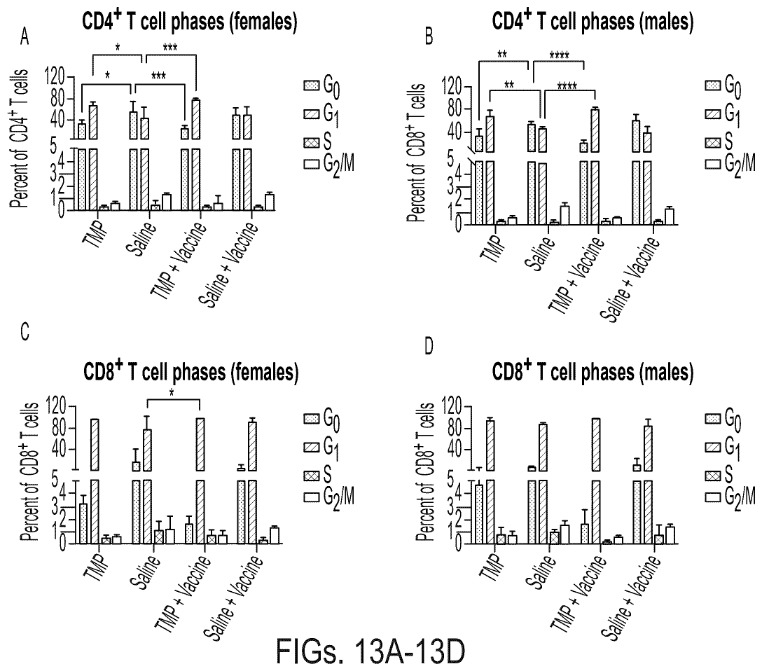

In comparison, on Day 17, relative to saline, TMP treatment decreased total T cells and CD4+ T cells of female mice (FIGS. 11A-11F). Similarly, on Day 38, TMP treated mice showed trends for decreased immune cell counts (FIGS. 12A-12F). Compared to saline-treated mice, immunosuppressed male and female mice showed increased CD4+ T cells in the G1 phase of the cell cycle and decreased CD4+ T cells in the G0 phase of the cell cycle on Day 38 (FIGS. 13A-13D). The observed increase of CD4+ T cells in the G1 phase of the cell cycle indicates stalling of cells in this phase of the cell cycle.

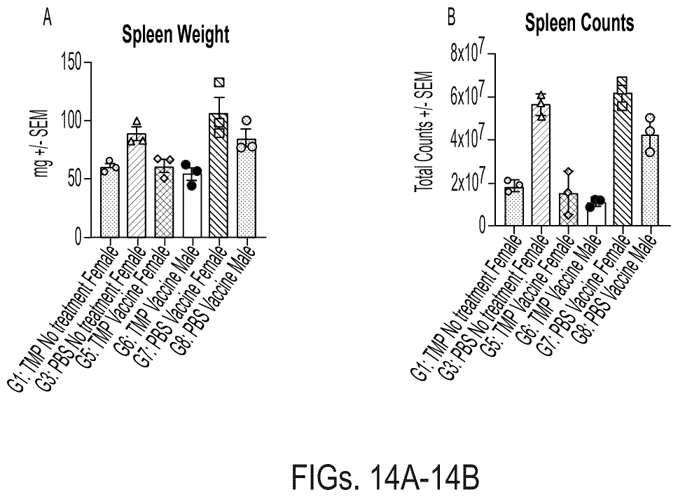

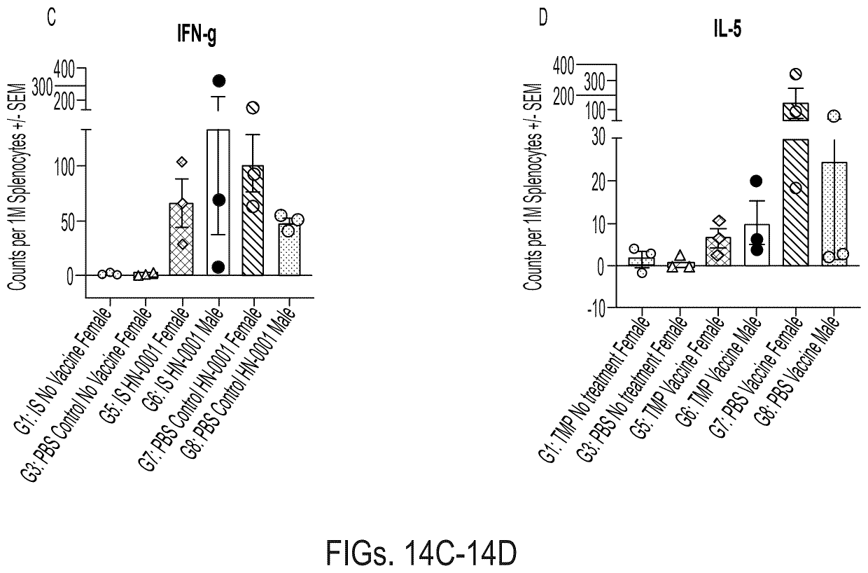

An analysis of spleen weights and splenocyte counts showed that both were reduced in immunosuppressed groups, indicating that the mice had drug-induced immunosuppression (FIGS. 14A-14D). ELISPOT analysis was also used to measure IFN-γ and IL-5+ T cells; the data showed increased IFN-γ and increased IL-5 from splenocytes of IS male mice.

Taken together, these results validate the IS mouse model and support its use as a surrogate for studying the effect of vaccines and/or therapeutics in subject's having a suppressed immune system.

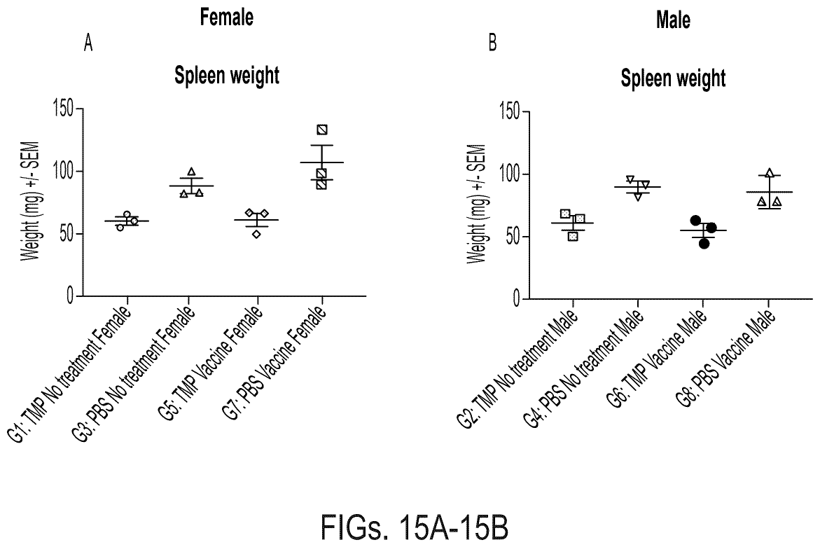

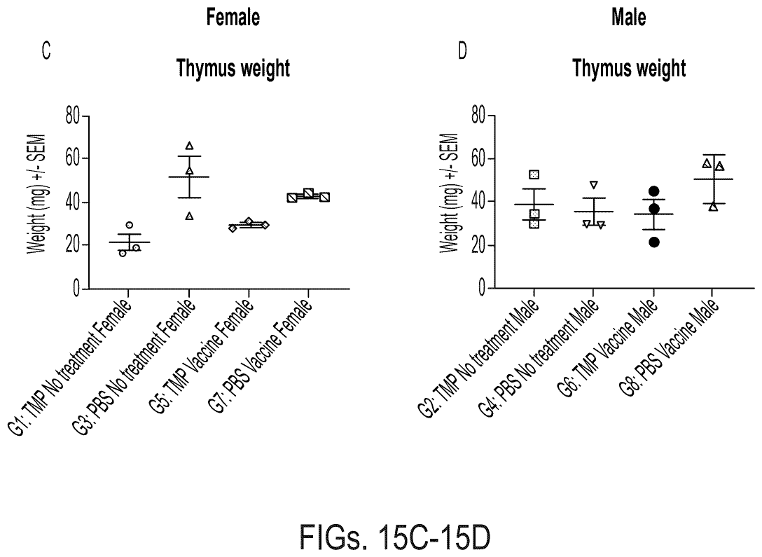

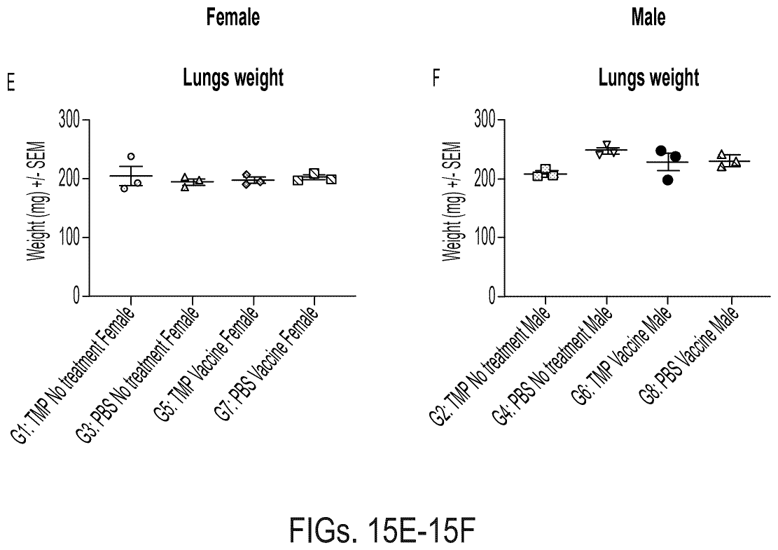

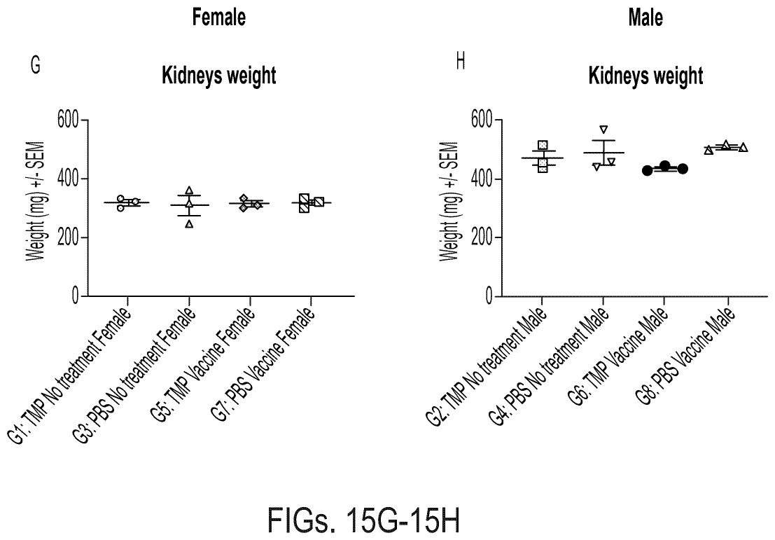

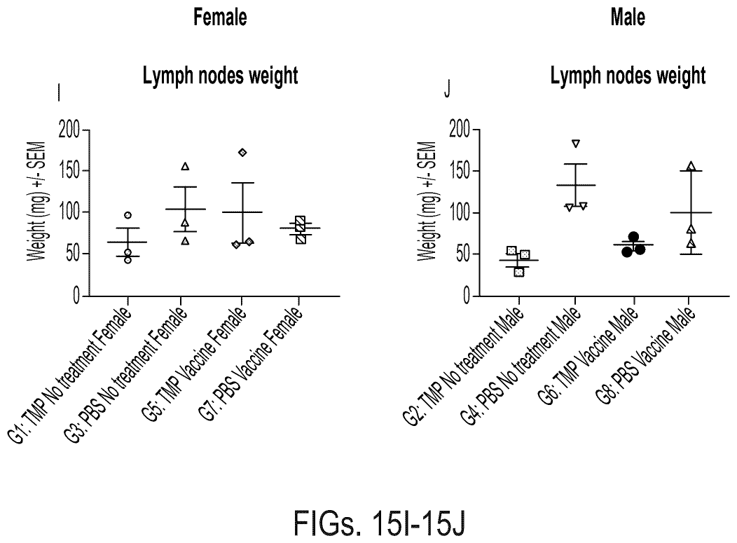

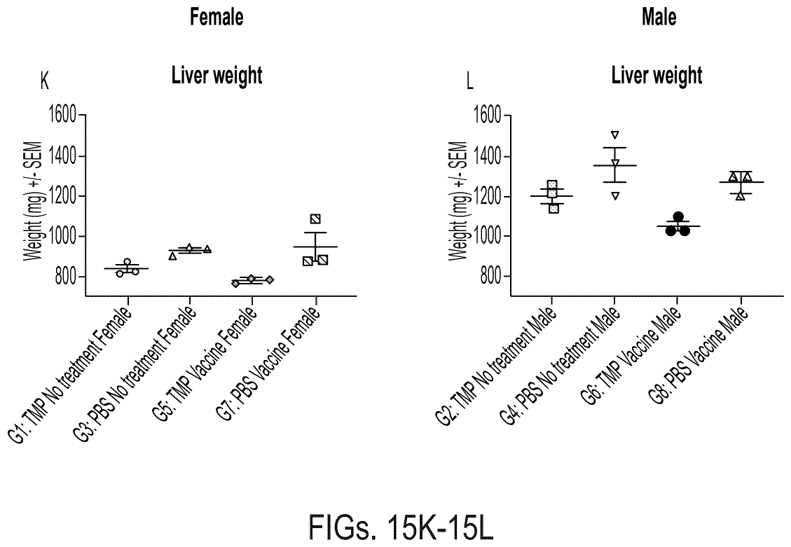

The IS mouse model was also used to test the efficacy of vaccines comprising an Sbi adjuvant, e.g., as described herein. Clinical observation of the animals throughout the study indicated that mice that were administered the exemplary vaccination comprising an Sbi adjuvant did not exhibit any adverse side effects as a result of vaccination. For example, immunization with the exemplary vaccine did not negatively impact bodyweights for either males or females, regardless of immune status (FIGS. 7A-7D). Moreover, vaccination did not cause any erythema or edema at the injection sites for any of the groups. Body temperatures of all animals were comparable irrespective of immunization or immunosuppression (FIGS. 8A-8B). Furthermore, as shown in FIGS. 15A-15L, most of the tissues analyzed had similar weights in immunosuppressed animals (with or without vaccination) as compared to immunocompetent animals.

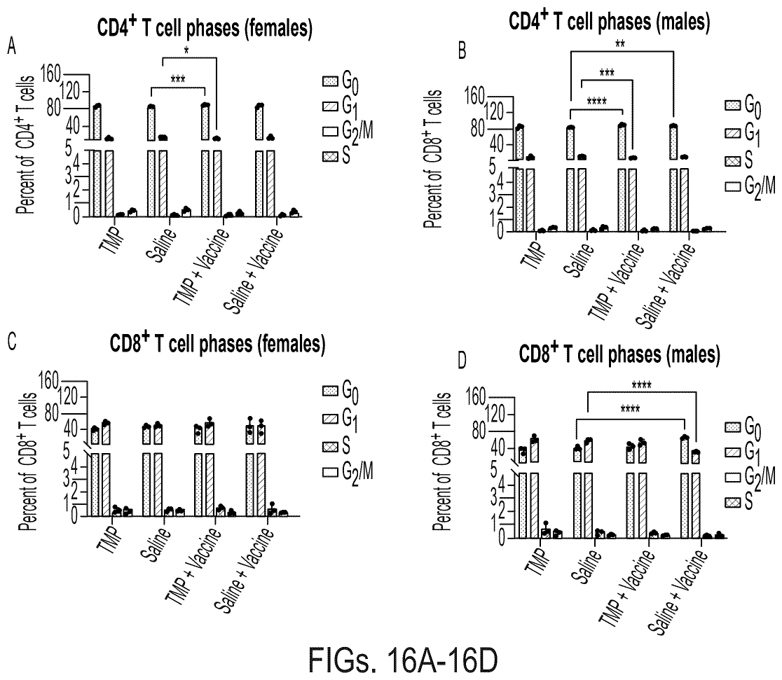

On Day 17, vaccination increased total T cells, CD4+ T cells, CD8+ T cells and B cells of saline-treated male mice, but not saline-treated females, and did not increase total T cells, CD4+ T cells, CD8+ T cells and B cells of immunosuppressed mice, regardless of sex. Immunosuppression in combination with vaccination did not affect counts of NK cells and neutrophils (FIGS. 11A-11F). Cell cycle phase data suggested that vaccination-induced increases in T cell counts were not associated with proliferation, as the percentages of cells in G1, S and G2/M phases did not increase (FIGS. 16A-16D).

Similarly, increased T cells and B cell counts were also observed on Day 38. (FIGS. 12A-12F). Vaccinated immunosuppressed mice showed the greatest increase in G1, relative to saline. CD8+ T cells percentages in G1 also increased in vaccinated, immunosuppressed female mice (FIGS. 13A-13D).

Together, these results indicate that vaccination with an exemplary Sbi-RBD fusion polyribonucleotide in immunosuppressed mice does not have an impact on immune cell counts.

To assess whether vaccination with antigens joined (e.g., directly or indirectly) to Sbi improve cellular response, ELISPOT analysis was used to measure IFN-γ and IL-5 T cells. The results indicated that the immunosuppressed vaccinated mice were able to elicit IFN-γ and IL5 T cell responses (FIGS. 14A-14D).

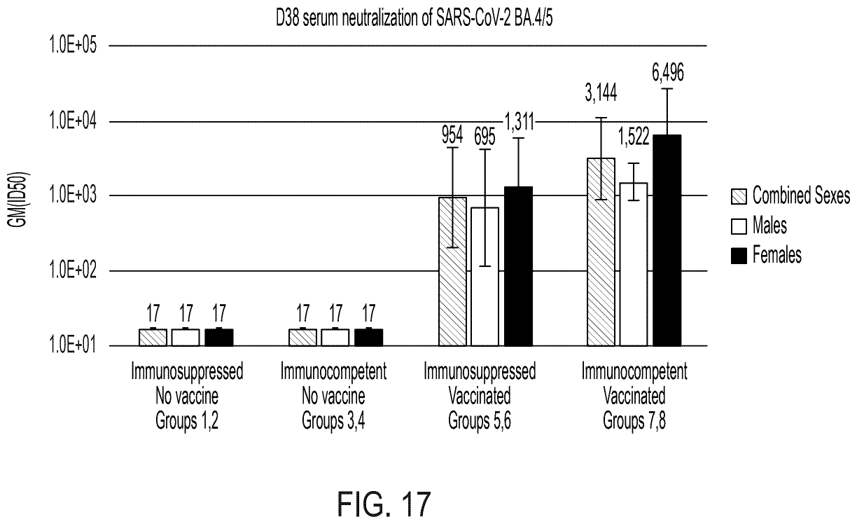

In addition, serum samples collected from these animals on Day 38 were analyzed for neutralizing titers. The drop in neutralizing antibody response from immunocompetent to immunosuppressed groups further confirms that TMP treatment impairs immune responses, as expected. Additionally, serum analysis indicated that the absolute magnitudes of the ID50s in immunosuppressed mice that were vaccinated were in the range of what is expected to be protective and were comparable to the immune response of the vaccinated immune competent mice (FIG. 17). This data shows that immunosuppressed mice are able to generate a functional immune response when vaccinated with an exemplary vaccine comprising an Sbi adjuvant.

Together, these results functionally validate the immunosuppression mouse model and demonstrate that vaccination of immunosuppressed mice with an exemplary Sbi-RBD fusion polyribonucleotide provides robust functional neutralizing antibody responses.

### Example 7: Improved Humoral Response Following Vaccination with Sbi Fusion in an Immunosuppressed Mouse Model

This Example describes the humoral immune response in wild-type (WT) mice or immunosuppressed (IS) mice vaccinated with an LNP formulation comprising: (1) an RNA encoding an RBD; or (2) an RNA encoding an exemplary fusion polypeptide with an Sbi(III-IV) joined (e.g., directly or indirectly) to an RBD.

### Methods

RNA synthesis: Transcription templates for RNA vaccines were synthesized as clonal genes (Integrated DNA Technologies).

RBD and RBD-Sbi templates were amplified with T7-AGG_fwd (gaattTAATACGACTCACTATAAGGcttgttctttttgcagaagc)(SEQ ID NO: 11) and 120 pA_rev (TTTTTTTTTTTTTTTTTTTTTTTTTTTTTTTTTTTTTTTTTTTTTTTTTTTTTTTTTTTTTTTTTTTTTTTTTTTTTTTTTTTTTTTT TTTTTTTTTTTTTTTTTTTTTTTTTTTTTTTTagaatgtgaagaaactttctttttattag) (SEQ ID NO: 12) using Herculase II polymerase (Agilent) with an annealing temperature of 50° C. Spike template was amplified with T7-pbSpike_fwd (GAATTTAATACGACTCACTATAAGGATAAACTAGTATTCTTCTGGTCC)(SEQ ID NO: 13) and pdSpike_pA_rev (TTTTTTTTTTTTTTTTTTTTTTTTTTTTTTTTTTTTTTTTTTTTTTTTTTTTTTTTTTTTTTTTTTTTTTAGTCATATGCTTTTTTT TTTTTTTTTTTTTTTTTTTTTTTGCTAGCTCCAGGGTG)(SEQ ID NO: 14) using Herculase II polymerase (Agilent) with an annealing temperature of 60° C.

PCR products were treated with DpnI (NEB) to digest plasmid template and then cleaned up using DNA Clean & Concentrator-25 (Zymo Research). 19.9 μL transcription mixes consisting of 1× HiScribe T7 High Yield buffer (NEB), 7.5 mM of each NTP, 7.5 mM CleanCap AG (TriLink Biotech), 2M betaine (ThermoSci), 20 mM MgCl2, and 0.1 μL/μL HiScribe T7 Polymerase Mix were added to 2.1 uL DNA solution consisting of 200 ng T7 template in nuclease free H2O. Transcription was carried out for 2 hr at 50° C. The transcribed RNAs were purified using the 500 μg capacity Monarch RNA Cleanup Kit, treated with DNAse I and Alkaline Phosphatase (Promega) for 5 min at 37° C. and purified using 500 μg Monarch columns. Concentrations were determined using a NanoDrop spectrophotometer.

LNP formulation: Formulations of RNA in lipid nanoparticles (RNA-LNPs) were prepared at HelixNano using the NanoAssemlr Ignite microfluidic mixer (Precision Nanosystems). GenVoy-ILM lipid mixture (Precision Nanosystems) was diluted to 12.5 mM in anhydrous ethanol and combined with an aqueous solution of RNA (0.14 mg/mL) in PNI buffer (Precision Nanosystems), using the manufacturer-recommended formulation parameters. Formulations were immediately diluted 30:1 in phosphate-buffered saline (pH 7.4) and concentrated using Amicon centrifugation filters (MilliporeSigma UFC901008). Formulations were diluted with sterile PBS to the appropriate concentration to allow for a dose of 0.25 ug or 2.5 ug RNA in 50 uL per animal. Formulations were shipped on wet ice, stored at 4° C. upon receipt, and used for in vivo studies within 21 days of production.

In vivo methods: Animals: All animal experiments were carried out in accordance with animal use protocol AUP #20-0723-MR-2: Pharmacokinetic, Pharmacodynamic and Biodistribution Studies in Laboratory Rodents, set forth by Aragen (Aragen Bioscience Inc, Morgan Hill, California, USA). Female BALB/c mice (aged 7-9 weeks) were purchased from Charles River Laboratories (Wilmington, MA, USA) and housed at Aragen. Mice were provided standard feed and water ad libitum. Mice were acclimated for at least 3 days before the initiation of study. Mice were weighed and distributed into 15 groups of n=5 such that the average weight of each group were similar.

Beginning on Day −2, two days prior to study initiation, mice were treated with TMP, or PBS via oral gavage. Treatment was continued daily for a total of 35 days ending on d33.

Immunosuppressive drua cocktail (TMP) preparation: TMP was prepared as described in Example 6. Prior to dosing the drugs in the TMP preparation were combined and given in a total volume of 200 uL, completed with saline. For each group of n=5 mice, the average body weight taken at the beginning of the week will be used to calculate the amount of TMP cocktail received for the remainder of the week.

Vaccination and sample collection: Mice were vaccinated (intramuscular injection in alternating quadriceps muscle, 50 uL dose volume) twice at an interval of 21 days; the first injection was administered on the first day of the study (also referred to as Day 0) and the second injection was administered 21 days later (also referred to as Day 21). Control groups were dosed with sterile PBS. One group received additional doses 14 days after the first injection (also referred to as Day 14) and 28 days after the first injection (also referred to as Day 28), making for a weekly vaccination regimen.

Mice were euthanized 33 days after the start of the study (also referred to as Day 33). Terminal blood was collected by means of cardiac exsanguination, processed to serum, and aliquoted for −80 C storage and cold shipment. Mucosal washes of the vaginal mucosa were performed immediately following euthanasia and blood collection. A p20 pipet was used to introduce 20 μl sterile PBS into the vagina. The fluid was pipetted back and forth within the organ 8 times before transferring sample volume into a 1.5 mL tube. This process was repeated a total of 3 times per animal, using a clean p20 tip each time, yielding a final pooled collection volume of 60 μl per animal. Wash samples were stored on wet ice until preparation for storage. Wash samples were spun down in a centrifuge at 300 rcf for 7 minutes to pellet any cells out of the supernatant. The supernatant was then transferred into sterile 1.5 mL tubes and shipped overnight on wet ice. Samples were assayed immediately upon receipt and were not frozen.

Anti-spike IqG ELISA: Serum IgG response against SARS-CoV-2 Spike was assessed using an indirect ELISA protocol adapted from Amanat, et al. (Nat Med 26: 1033-1036, 2020). 96 well Thermo Maxisorp plates (Thermo Fisher Scientific 442404) were coated with 50 μl per well of a 2 μg/ml solution of SARS-CoV-2 (2019-nCoV) Spike S1+S2 ECD-His recombinant protein (Sino Biological #40589-V08B¹) in PBS and incubated at 4° C. overnight. Plates were then washed three times with 300 μl of 0.1% Tween 20 in PBS (PBS-T) using an automated plate washer (BioTek) and blocked with 200 μl per well of SuperBlock PBS blocking buffer (Thermo Fisher Scientific) for 1 hr at room temperature. Serial dilutions of serum samples were prepared in a 1:3 dilution of Superblock in PBS. 100 μl of each serial dilution was added to the plates and incubated for 2 hr at room temperature. The wells were then washed three times using PBS-T, as previously described. 50 μl of a 1:3,000 dilution of goat anti-mouse IgG horseradish peroxidase-conjugated secondary antibody (Sigma-Aldrich, AP127P) in a 1:3 dilution of Superblock in PBS was added to all wells and incubated at room temperature for 1 hr. Plates were again washed three times in PBS-T. 100 μl SIGMAFAST OPD (Sigma-Aldrich) solution was added to each well and incubated for 10 min at room temperature. Reactions were stopped by addition of 50 μl per well of 2N hydrochloric acid. Optical density was measured at 490 nm using a GloMax Discover (Promega) plate reader.

Reported titers were determined by taking the last dilution before the signal dropped below 3 standard deviations above the average of the signal from the untreated control serum at the same dilution. The last dilution was taken as the titer if the signal never dropped below this threshold. If no signal above the threshold was detected, the value in the dilution series before the least-dilute sample tested was used. The geometric mean (GMT) and geometric standard deviations (positive and negative GSD) were calculated across all animals with a treatment group (n=5).

Anti-RBD IgG MSD assay: Serum IgG response against SARS-CoV-2 RBD was assessed using Meso Scale Discovery (MSD) V-PLEX SARS-CoV-2 Serology panel 28. All listed MSD reagents were provided in said kit. Plates were blocked with 150 μL/well of MSD Blocker A at room temperature, shaking at 700 rpm on an orbital plate mixer for 1 hour (Heidolph Instruments, Titramax 1000). Plates were then washed 3 times with 150 μL/well 1×MSD Wash buffer using an automated plate washer (BioTek, 405-TS) and were thoroughly tapped dry. Serum thawed from −80° C. storage was prepared at 1:100,000 and 1:1,000,000 dilutions in MSD Diluent-100 and added to MSD plates in duplicate at 50 μL/well. A 1 hour incubation and plate wash step were performed as previously described. Secondary detection antibody, anti-mouse IgG MSD GOLD SULFO-TAG, was prepared according to kit protocol as a 1:200 dilution in MSD Diluent-100 and was added 50 μL/well. Another 1 hour incubation and plate wash step were performed as previously described. Plate wash step was carried out as previously described. To read plates, 150 μL/well of MSD GOLD Read Buffer B was added to plates and signal was detected using the MESO QuickPlex SQ 120 MM instrument.

Results are reported as the mean detected signal within a well, averaged across replicates, using relative light units (RLU). Increased signal indicates a larger quantity of antigen specific IgG antibody in a given sample. Signal was averaged across n=5 samples per group. Standard deviation was calculated across n=5 samples per group.

Pseudovirus neutralization assay: Neutralization titers were measured by PPD using a lentivirus pseudotyped with full length GFP SARS-CoV-2 BA.4/5 Spike protein and a 293T-hsACE2 cell line. Serum was diluted at 1:50 and used to make a 3-fold dilution series ranging out to 1:12150, or 1:328050 in a follow up re-assay where samples were saturated out to 1:12150. Dilution preparation was automated using a Hamilton Star instrument (Hamilton Robotics).

Cells were plated with serum dilutions then infected with lentivirus for a period of 48 hours. Serum dilutions were assayed in replicate. Reference standards and 3 Quality Control Standards were run in duplicate on all plates. Readout was performed using single cell enumeration quantifying focus forming units (FFU) per well with the Cytostation 5 (Tecan).

Pseudotype neutralization titers were calculated as inverse geometric mean titers (inverse GMT) across n=5 samples per group. Positive and negative geometric standard deviation (GSD) was calculated across n=5 samples per group.

### Results

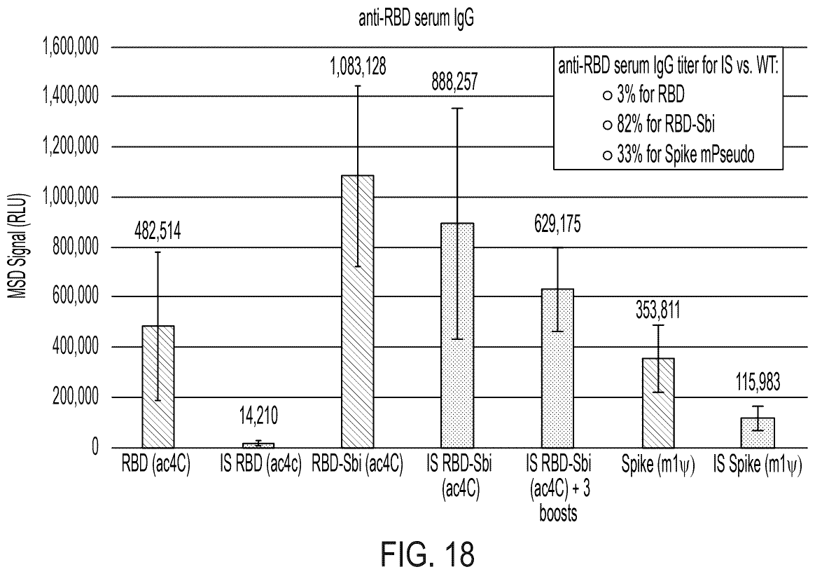

As demonstrated by anti-Spike ELISA and anti-RBD MSD analysis in FIG. 18, daily oral administration of TMP significantly dampened the humoral immune response of vaccinated mice (immunosuppressed (IS) mice) as compared to vaccinated WT mice. When vaccinated with an RBD RNA/LNP vaccine alone, IS mice showed a mere 3.2% induction of anti-RBD serum IgG antibody relative to vaccinated WT mice (compare first and second bars). With the addition of an exemplary Sbi fusion, IS treated mice maintained 82% of the anti-RBD IgG antibody measured in WT mice (compare third and fourth bars). Comparatively, only 33% of this response was maintained in IS mice vaccinated with a control Spike RNA/LNP vaccine with N1-methylpseudouridine modified nucleosides (a commonly used modified nucleoside; compare sixth and seventh bars). This data demonstrates that vaccination with an RNA vaccine comprising Ac4C modified nucleosides outperformed vaccination with an RNA having N1-methylpseudouridine modified nucleosides. Additionally, a traditional two dose vaccine regimen administered at an interval of 21 days yielded a greater humoral response as compared to a repeated weekly boosting regimen totaling 4 doses (compare fourth and fifth bars).

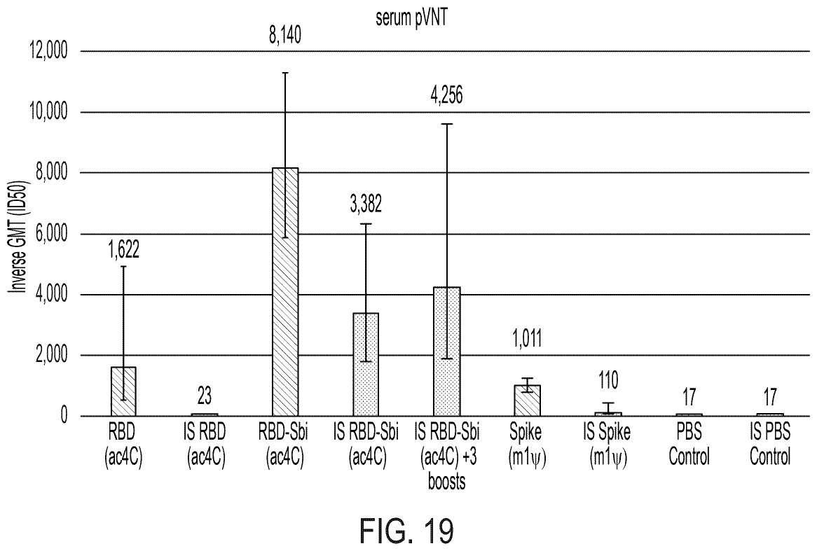

Pseudovirus neutralization titers (pVNT) were nearly ablated in RBD vaccinated IS treated mice, as well as full Spike (mPseudo) vaccinated IS mice, falling to an ID50 of 23 and 110, respectively (FIG. 19). In contrast, vaccination of IS mice with an exemplary RBD-Sbi vaccine demonstrated over 30× neutralizing antibody titer at an ID50 of 3,382 following 2 doses and an ID50 of 4,256 following a total of 4 weekly doses. The weekly dosing regimen showed higher pseudovirus neutralization titers compared to the 2-dose regimen despite having lower anti-RBD and anti-Spike serum IgG. In some embodiments, this may be related to increased intragroup variance or the study termination falling only 5 days after the weekly boost group's final vaccination, compared to 14 days after the 2-dose group's final vaccination.

Taken together, this data demonstrates that vaccination with antigens joined (e.g., directly or indirectly) to Sbi can enhance humoral responses and provide neutralizing titers in immunosuppressed subjects. In some embodiments, this data supports the use of Sbi fusions for enhancing the immunogenicity of an antigen, and/or for stimulating an immune response against an antigen, in immunosuppressed subjects.

### Example 8: SbI(III-IV) Fusion to an Exemplary Influenza Antigen Increases Humoral Immune Response Against the Viral Antigen

This Example describes increased antigen-specific antibody titers in serum and vaginal washes from mice vaccinated with lipid nanoparticles (LNPs) comprising RNAs encoding fusion polypeptides comprising an Sbi(III-IV) joined (e.g., directly or indirectly) to an exemplary influenza antigen.

### Material and Methods

gBlock amplification: Each gBlock template was amplified with T7-AGG_fwd (gaattTAATACGACTCACTATAAGGcttgttctttttgcagaagc)(SEQ ID NO: 11) and 120 pA_rev (TTTTTTTTTTTTTTTTTTTTTTTTTTTTTTTTTTTTTTTTTTTTTTTTTTTTTTTTTTTTTTTTTTTTTTTTTTTTTTTTTTTTTTTT TTTTTTTTTTTTTTTTTTTTTTTTTTTTTTTTagaatgtgaagaaactttctttttattag) (SEQ ID NO: 12) using Herculase II polymerase (Agilent) with an annealing temperature of 50° C.

RNA synthesis: PCR products were cleaned up using a 0.8× ratio of SPRISelect beads to PCR reaction volume. 19.9 μL transcription mixes consisting of 1× HiScribe T7 High Yield buffer (NEB), 7.5 mM of each NTP, 7.5 mM CleanCap AG (TriLink Biotech), 2M betaine (ThermoSci), 20 mM MgCl2, and 0.1 μL/μL HiScribe T7 Polymerase Mix were added to DNA solution consisting of 200 ng T7 template in nuclease free H2O. Transcription was carried out for 2 hr at 50° C. Transcribed RNAs were purified using the 500 μg capacity Monarch RNA Cleanup Kit, treated with DNAse I, and purified again using 500 μg-capacity Monarch columns. mRNAs were then treated with Calf Intestine Alkaline Phosphatase (Promega) and DNaseI (NEB) for 5 min at 37° C. and purified using 500 μg Monarch columns. Concentrations were determined using a NanoDrop spectrophotometer.

LNP formulation: Formulations of RNA in lipid nanoparticles (RNA-LNPs) were prepared using a NanoAssemlr Ignite microfluidic mixer (Precision Nanosystems). GenVoy-ILM lipid mixture (Precision Nanosystems) was diluted to 12.5 mM in anhydrous ethanol and combined with an aqueous solution of RNA (0.14 mg/mL) in PNI buffer (Precision Nanosystems), using the manufacturer-recommended formulation parameters. Formulations were immediately diluted 30:1 in phosphate-buffered saline (pH 7.4) and concentrated to 0.4 mg/mL using Amicon centrifugation filters (MilliporeSigma UFC901008), and characterized using a Stunner instrument (Unchained Labs). Formulations were stored at 4° C. and used for in vivo studies within 14 days.

Animal vaccination: All animal experiments were carried out in accordance with the guidelines set forth by Charles River Accelerator Development Lab (CRADL) and were approved by the CRADL Institutional Animal Care and Use committee. Female BALB/C mice (7-9 weeks old) were purchased from Charles River Laboratories and housed at CRADL. Mice were acclimated for at least 3 days before the initiation of a study. RNA-LNP formulations were brought to room temperature, and diluted 1:1 in PBS prior to injection. On Day 1, mice were injected in the right quadriceps with 50 μL RNA-LNP formulation (10 μg RNA dose), and on Day 26 mice were injected in the left quadriceps with 50 μL RNA-LNP formulation (10 μg RNA dose). Untreated control mice received no injections. Mice were euthanized on Day 34, at which time blood was collected via intracardiac stick, and mucosal samples (in 60 uL PBS) were collected via vaginal wash. Serum was separated from blood in MiniCollect serum separator tubes (Greiner Bio-One 450472) by centrifugation at 4° C., 1200×g, for 10 minutes. Mucosal samples were stored at 4° C. and used to determine anti-antigen IgA titers via ELISA the next day. Serum was stored at −80° C. and used to evaluate anti-antigen IgG titers via ELISA within two days of collection.

Anti-antigen serum IgG ELISA assay: 96-well Immulon 4 HBX plates (Thermo Fisher Scientific) were coated with 50 μl per well of a 2 μg/ml solution of recombinantly produced and purified Influenza antigen protein (Sino Biological) in PBS at 4° C. overnight. Plates were washed three times with 300 μl of 0.1% Tween 20 in PBS (PBS-T), then were blocked for 1 hour with 200 μl per well of SuperBlock Blocking Buffer (ThermoFisher). Serial dilutions of serum were prepared in 3× diluted SuperBlock, and 100 μl of each was added to the plates for 2 hours at room temperature. Wells then were washed three times with PBS-T as before. Wells were then incubated with 50 μl of a 1:3,000 dilution of goat anti-mouse IgG horseradish peroxidase-conjugated secondary antibody (Sigma) in 3×-diluted SuperBlock at room temperature for 2 hours. Plates were again washed three times with PBS-T. 100 μl SIGMAFAST OPD (Sigma-Aldrich) solution was added to each well and plates were incubated for 10 min at room temperature. Reactions were stopped by addition of 50 μl per well of 3 M hydrochloric acid. Optical density was measured at 490 nm using a GloMax Discover (Promega) plate reader. End-point titers were determined by taking the last dilution before the signal dropped below three standard deviations above the average of the signal from the untreated control serum at the same dilution. The last dilution was taken as the titer if the signal never dropped below this threshold. If no signal above the threshold was detected, the value in the dilution series before the least-dilute sample tested was used.

### Results

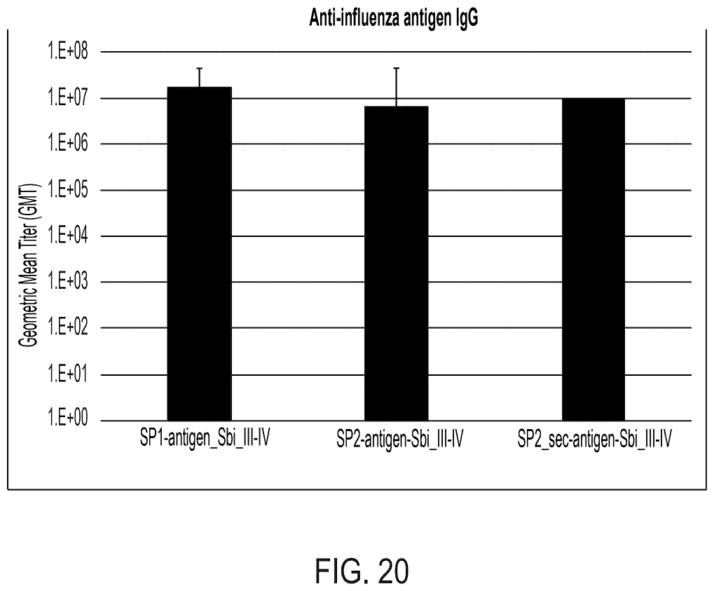

An ELISA serology assay measuring serum IgG antibodies against the exemplary antigen was performed to quantify humoral immune responses induced by RNAs encoding an Sbi(III-IV) polypeptide fused to the exemplary influenza antigen. Results depicted in FIG. 20 show that antigen-specific serum IgG titer is consistently high for vaccines incorporating Sbi_III-IV together with chemically modified nucleotides, N4-acetylcytidine (“ac4C′” and 5-hydroxymethyluridine (“5-hmU”). The data further demonstrates minimal changes in antigen-specific serum IgG differs with the different exemplary signal peptides (e.g., exemplary signal peptide 1 (“SP1”), exemplary signal peptide 2 (“SP2”), or exemplary signal peptide 2 joined (e.g., directly or indirectly) to an exemplary secretion sequence (“SP2_sec”)).

Taken together, this data demonstrates that vaccination with RNAs having chemical modifications such as ac4C and/or 5-hmU and encoding an Sbi(III-IV) polypeptide fused to an exemplary influenza antigen generated meaningful humoral immune responses in vivo in mice. This data supports the utility of using polyribonucleotides having chemical modifications, e.g., ac4C and/or 5-hmU, and encoding fusion polypeptides comprising Sbi domains (e.g., Sbi III and/or IV) and one or more antigens for enhancing the immunogenicity of an antigen, and/or for stimulating an immune response against an antigen in a subject.

### EQUIVALENTS

Those skilled in the art will recognize, or be able to ascertain using no more than routine experimentation, many equivalents to the specific embodiments of the invention described herein. It is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Further, it should also be understood that any embodiment or aspect of the invention can be explicitly excluded from the claims, regardless of whether the specific exclusion is recited in the specification. The scope of the present invention is not intended to be limited to the above Description, but rather is as set forth in the claims that follow.

1. A method comprising, delivering to an immunosuppressed subject a composition comprising a polynucleotide encoding a fusion polypeptide, wherein the fusion polypeptide comprises:
(a) one or more antigens comprising an epitope of a target protein antigen, and
(b) a complement C3d-binding polypeptide from an immunoglobulin-binding protein (Sbi) of Staphylococcus aureus.

2. The method of claim 1, wherein at least one dose is administered.

3. The method of claim 2, wherein the at least one dose is administered in an effective amount to:
(i) induce an immune response against the antigen in the subject,
(ii) stimulate B cells in a subject, or
(iii) both (i) and (ii).

4. The method of claim 3, wherein B cells are stimulated without inducing a T cell response.

5. The method of claim 3, wherein B cells are stimulated in the absence of T cells.

6. The method of claim 3, wherein B cells are stimulated in the presence of T cells, wherein the T cells have a reduced ability to induce an immune response.

7. A method for enhancing the immunogenicity of an antigen, comprising administering to an immunosuppressed subject, a composition comprising a polynucleotide encoding a fusion polypeptide, wherein the fusion polypeptide comprises:
(a) one or more antigens comprising an epitope of a target protein antigen, and
(b) a complement C3d-binding polypeptide from an immunoglobulin-binding protein (Sbi) of Staphylococcus aureus.

8. The method of claim 7, wherein enhancing immunogenicity of an antigen comprises stimulating an immune response against the antigen or stimulating a humoral immune response.

9-10. (canceled)

11. A method for stimulating an immune response against an antigen, comprising administering to an immunosuppressed subject, a composition comprising a polynucleotide encoding a fusion polypeptide, wherein the fusion polypeptide comprises:
(a) one or more antigens comprising an epitope of a target protein antigen, and
(b) a complement C3d-binding polypeptide from an immunoglobulin-binding protein (Sbi) of Staphylococcus aureus.

12. The method of claim 1, wherein the composition comprises a pharmaceutical composition.

13. The method of claim 1, wherein the polynucleotide comprises: a modified nucleobase, a modified deoxyribose, a modified ribose, a modified backbone, or any combination thereof.

14. The method of claim 1, wherein the polynucleotide is or comprises RNA or DNA.

15. The method of claim 1, wherein the immunosuppressed subject has received, is receiving, or will be receiving one or more transplants.

16. The method of claim 15, wherein the one or more transplants is an organ transplant.

17. The method of claim 15, wherein the one or more transplants is a cell transplant.

18. The method of claim 15, wherein the one or more transplants is a tissue transplant.

19. The method of claim 1, wherein the immunosuppressed subject has received, is receiving or will be receiving one or more dialysis treatments.

20. The method of claim 1, wherein the immunosuppressed subject has one or more disorders.

21. The method of claim 20, the one or more disorders comprises: a rare disease, lung disease, an autoimmune disease, liver disease, kidney disease, a cardiovascular disease, a blood disorder, a neurologic disease, an immunodeficiency disorder, a cancer, or any combination thereof.

22. The method of claim 1, wherein the immunosuppressed subject has received or is receiving an immunosuppressive therapy.

23. The method of claim 22, wherein the immunosuppressive therapy comprises: an organ transplant conditioning regimen, a therapy that suppresses an immune system (e.g., an antibody therapy), a B-cell targeting therapy, a T-cell targeting therapy, a chemotherapy, a radiation therapy, a cancer therapy, a treatment for an inflammatory disease and/or a treatment for an autoimmune disease.

24-34. (canceled)

35. A polynucleotide encoding a fusion polypeptide, wherein the fusion polypeptide comprises:
(a) one or more antigens comprising an epitope of a target protein antigen, and
(b) a complement C3d-binding polypeptide from an immunoglobulin-binding protein (Sbi) of Staphylococcus aureus,
wherein the polynucleotide comprises one or more modified ribonucleotides comprising: a modified nucleobase, a modified deoxyribose, a modified ribose, a modified backbone, or any combination thereof.

36. (canceled)

37. The polynucleotide of claim 35, wherein the complement C3d-binding polypeptide is or comprises one or both of domain III and domain IV of the Sbi of Staphylococcus aureus, or a functional fragment or a variant thereof.

38. The polynucleotide of claim 35, wherein the one or more antigens comprise: one or more antigen variants or one or more antigen fragment variants that comprise at least one modified amino acid compared to a target protein antigen.

39. (canceled)

40. The polynucleotide of claim 35, wherein the one or more antigens comprise:
(i) an infectious disease antigen, wherein the infectious disease antigen is or comprises viral antigen, a bacterial antigen, a fungal antigen, or combinations thereof, or
(ii) a cancer antigen.

41-43. (canceled)

44. The polynucleotide of any one of claim 35, wherein the one or more modified ribonucleotides comprises a nucleoside comprising: N4-acetylcytidine, 5-hydroxymethyluridine, N1-methylpseudouridine, pyridin-4-one ribonucleoside, 5-aza-uridine, 6-aza-uridine, 2-thio-5-aza-uridine, 2-thio-uridine (s2U), 5-methyl cytidine (m5C), 5-aza-cytidine, 6-aza-cytidine, pseudoisocytidine, 3-methyl-cytidine (m3C), 5-formyl-cytidine (f5C), N4-methyl-cytidine (m4C), 2-amino-purine, 2, 6-diaminopurine, 2-amino-6-halo-purine, 6-halo-purine, inosine (I), 1-methyl-inosine (ml I), wyosine (imG), methylwyosine (mimG), 5-hydroxycytidine, 5-hydroxymethylcytidine, 5-carboxycytidine, 5-methoxycytidine, 5-propynylcytidine, 2-thiocytidine, 5-hydroxyuridine, 5-methyluridine, 5,6-dihydro-5-methyluridine, 2′-O-methyluridine, 2′-O-methyl-5-methyluridine, 2′-fluoro-2′-deoxyuridine, 2′-amino-2′-deoxyuridine, 2′-azido-2′-deoxyuridine, 4-thiouridine, 5-carboxyuridine, 5-carboxymethylesteruridine, 5-formyluridine, 5-methoxyuridine, 5-propynyluridine, 5-bromouridine, 5-iodouridine, 5-fluorouridine, pseudouridine, 2′-O-methyl-pseudouridine, N1-hydroxypseudouridine, 2′-O-methyl-Ni-methylpseudouridine, N1-ethylpseudouridine, N1-hydroxymethylpseudouridine, ara-uridine, N6-methyladenosine, 2-aminoadenosine, 3-methyladenosine, 7-deazaadenosine, 8-oxoadenosine, thienoguanosine, 7-deazaguanosine, 8-oxoguanosine, 6-O-methylguanine, or any combination thereof.

45. The polynucleotide of claim 35, wherein the one or more modified ribonucleotides comprise ribonucleotides that comprise a 2′-O-acetylated ribose.

46-49. (canceled)

50. A fusion polypeptide encoded by the polynucleotide of claim 35.

51. An expression vector comprising the polynucleotide of claim 35.

52-55. (canceled)

56. A cell comprising the polyribonucleotide of claim 35.
